(19) United States
(12) Patent Application Publication
(10) Pub. No.: US 2009/0221664 A1
(43) Pub. Date: Sep. 3, 2009

(54) PHARMACEUTICAL COMPOSITIONS OF MUSCARINIC RECEPTOR ANTAGONISTS

(76) Inventors: Abhijit Ray, New Delhi (IN); Sunanda G. Dastidar, New Delhi (IN); Rajkumar Shirumalla, New Delhi (IN); Shivani Malhotra, New Delhi (IN)

Correspondence Address: RANBAXY INC.;INTELLECTUAL PROPERTY DEPT., 600 COLLEGE ROAD EAST, SUITE 2100, PRINCETON, NJ, 08540, US

(21) Appl. No.: 12/090,805

(22) PCT Filed: Oct. 19, 2006
(86) PCT No.: PCT/IB2006/002930

§ 371 Date: Oct. 1, 2008

(30) Foreign Application Priority Data
Oct. 19, 2005 (IN) 2794/DEL/2005

(51) Int. Cl. A61K 31/403

(52) U.S. Cl. 514/412

(57) ABSTRACT

Provided herein are pharmaceutical compositions comprising one or more muscarinic receptor antagonists (“MRA”), and at least one additional active ingredients selected from one or more β2-agonists, p38 MAP kinase inhibitors, PDE-IV inhibitors, corticosteroids or a mixture thereof and optionally one or more pharmaceutically acceptable carriers, excipients or diluents. In addition, methods of treating autoimmune, inflammatory or allergic diseases or disorders are provided.

### TECHNICAL FIELD OF THE INVENTION

Provided herein are pharmaceutical compositions comprising one or more muscarinic receptor antagonists (“MRA”) and at least one additional active ingredient selected from one or more β2-agonists, p38 MAP kinase inhibitors, PDE-IV inhibitors, corticosteroids or mixtures thereof and optionally one or more pharmaceutically acceptable carriers, excipients or diluents. In addition, methods of treating autoimmune, inflammatory or allergic diseases or disorders are provided.

### BACKGROUND OF THE INVENTION

Muscarinic receptors, members of the G Protein Coupled Receptors (GPCRs), are composed of a family of 5 receptor sub-types (M₁, M₂, M₃, M₄ and M₅) and are activated by the neurotransmitter acetylcholine. These receptors are widely distributed on multiple organs and tissues and are critical to the maintenance of central and peripheral cholinergic neurotransmission. The regional distribution of these receptor sub-types in the brain and other organs has been documented. For example, the M₁ subtype is located primarily in neuronal tissues such as cerebral cortex and autonomic ganglia, the M₂ subtype is present primarily in the heart where it mediates cholinergically induced bradycardia, and the M₃ subtype is located primarily on smooth muscle and salivary glands (Nature, 323, p. 411 (1986); Science, 237, p. 527 (1987)).

The biological potentials of modulating muscarinic receptor subtypes by ligands in different disease conditions, such as Alzheimer's Disease, pain, urinary disease condition, chronic obstructive pulmonary disease, and the like, are described (Current Opinions in Chemical Biology, 3, p. 426 (1999), as well as in Trends in Pharmacological Sciences, 22, p. 409 (2001) by Eglen et al.).

Therapeutic opportunities for muscarinic receptors in the central nervous system and elaborates on muscarinic receptor structure and function, pharmacology and their therapeutic uses are described (J. Med. Chem., 43, p. 4333 (2000), by Felder et al).

The pharmacological and medical aspects of the muscarinic class of acetylcholine agonists and antagonists are described (Molecules, 6, p. 142 (2001)).

The recent developments on the role of different muscarinic receptor subtypes using different muscarinic receptor of knock out mice are described (Birdsall et al., Trends in Pharmacological Sciences, 22, p. 215 (2001)).

Muscarinic agonists such as muscarine and pilocarpine and antagonists such as atropine have been known for over a century, but little progress has been made in the discovery of receptor subtype-selective compounds, making it difficult to assign specific functions to the individual receptors. Although classical muscarinic antagonists such as atropine are potent bronchodilators, their clinical utility is limited due to high incidence of both peripheral and central adverse effects such as tachycardia, blurred vision, dryness of mouth, constipation, dementia, etc. Subsequent development of the quarterly derivatives of atropine such as ipratropium bromide are better tolerated than parenterally administered options, but most of these are not ideal anti-cholinergic bronchodilators, due to lack of selectivity for muscarinic receptor sub-types, resulting in dose-limiting side-effects such as thirst, nausea, mydriasis and those associated with the heart such as tachycardia mediated by the M₂ receptor.

The pharmacology of the lower urinary tract infections are described (Annual Review of Pharmacological Toxicol. 41, p. 691 (2001)). Although anti-muscarinic agents, such as oxybutynin and Tolterodine, which act non-selectively on muscarinic receptors have been used for many years to treat bladder hyperactivity, the clinical effectiveness of these agents has been limited due to side effects such as dry mouth, blurred vision and constipation. Tolterodine is considered to be generally better tolerated than oxybutynin. (Steers et al., in Curr. Opin. Invest. Drugs, 2, 268; Chapple et al, in Urology, 55, 33; Steers et al., Adult and Pediatric Urology, ed. Gillenwatter et al., pp 1220-1325, St. Louis, Mo.; Mosby. 3^{rd} Edition (1996)).

Compounds having antagonistic activity against muscarinic receptors have been described in Japanese patent application Laid Open Number 92921/1994 and 135958/1994; WO 93/16048; U.S. Pat. No. 3,176,019; GB 940,540; EP 0325 571; WO 98/29402; EP 0801067; EP 0388054; WO 9109013; U.S. Pat. No. 5,281,601. Also, U.S. Pat. Nos. 6,174,900, 6,130,232 and 5,948,792; WO 97/45414 describes 1,4-disubstituted piperidine derivatives; WO 98/05641 describes fluorinated, 1,4-disubstituted piperidine derivatives; and WO 93/16018 and WO96/33973 are other related references. U.S. Pat. No. 5,397,800 discloses 1-azabicyclo[2.2.1]heptanes. U.S. Pat. No. 5,001,160 describes 1-aryl-1-hydroxy-1-substituted-3-(4-substituted-1-piperazinyl)-2-propanones. WO 01/42213 describes 2-biphenyl-4-piperidinyl ureas. WO 01/42212 describes carbamate derivatives. WO 01/90081 describes amino alkyl lactam. WO 02/53564 describes novel quinuclidine derivatives. WO 02/00652 describes carbamates derived from arylalkyl amines. WO 02/06241 describes 1,2,3,5-tetrahydrobenzo(c)azepin-4-one derivatives.

A report in J. Med. Chem., 44, p. 984 (2002), describes cyclohexylmethyl piperidinyl triphenylpropioamide derivatives as selective M₃ antagonist discriminating against the other receptor subtypes.

However in view of the above, there remains a need for novel highly selective muscarinic receptor antagonists that can interact with distinct subtypes while avoiding the occurrence of adverse effects.

### SUMMARY OF THE INVENTION

In one general aspect, provided are pharmaceutical compositions comprising one or more muscarinic receptor antagonists (“MRA”) and at least one additional active ingredient selected from one or more β2-agonists, p38 MAP kinase inhibitors, PDE-IV inhibitors, corticosteroids or a mixture thereof and optionally one or more pharmaceutically acceptable carriers, excipients or diluents.

Suitable MRA can be one or more compounds having the structures of Formula I, II, or III, wherein:

a. Formula I is:

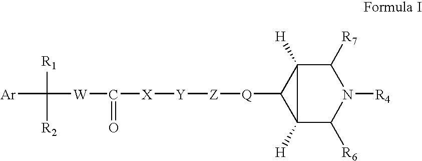

or a pharmaceutically acceptable salt, pharmaceutically acceptable solvate, ester, enantiomer, diastereomer, N-oxide, polymorphs, prodrugs or metabolite thereof, wherein
Ar represents an aryl or a heteroaryl ring having 1-2 heteroatoms independently selected from oxygen, sulphur or nitrogen, wherein
   the aryl or heteroaryl ring may be unsubstituted or substituted by one to three substituents independently selected from lower alkyl (C₁-C₄), lower perhalo alkyl (C₁-C₄), cyano, hydroxy, nitro, lower alkoxy (C₁-C₄), lower perhalo alkoxy (C₁-C₄), unsubstituted amino, N-lower alkyl (C₁-C₄), N-aryl amino, amino carbonyl, N-lower alkyl (C₁-C₄) or N-aryl amino carbonyl;
R₁ represents hydrogen, hydroxy, hydroxy methyl, substituted or unsubstituted amino, alkoxy, carbamoyl or halogen (e.g., fluorine, chlorine, bromine and iodine);
R₂ represents alkyl, (C₃-C₇) cycloalkyl ring, (C₃-C₇) cycloalkenyl ring, aryl, heterocyclic ring, or heteroaryl ring, wherein
   the heterocyclic ring or heteroaryl ring may have 1 to 2 heteroatoms independently selected from oxygen, sulphur or nitrogen, and
   the aryl or heteroaryl ring may be unsubstituted or substituted by one to three substituents independently selected from lower alkyl (C₁-C₄), lower perhalo alkyl (C₁-C₄), cyano, hydroxy, nitro, lower alkoxycarbonyl, halogen, lower alkoxy (C₁-C₄), lower perhalo alkoxy (C₁-C₄), unsubstituted amino, N-lower alkyl (C₁-C₄) or N-aryl amino, amino carbonyl, N-lower alkyl (C₁-C₄) or N-aryl amino carbonyl;
W represents (CH₂)ₚ, wherein p represents 0 to 1;
X represents oxygen, sulphur, —NR or no atom (i.e., a bond), wherein
   R represents hydrogen or (C₁₋₆) alkyl;
Y represents CHR₅CO or (CH₂)_{q}, wherein
   R₅ represents hydrogen or methyl, and
   q represents 0 to 4;
Z represents oxygen, sulphur, or NR₁₀, wherein
   R₁₀ represents hydrogen, or C₁₋₆ alkyl;
Q represents (CH₂)ₙ, CHR₈ or CH₂CHR₉, wherein
   n represents 0 to 4,
   R⁸ represents H, OH, C₁₋₆, alkyl, C₁₋₆ alkenyl, or C₁₋₆ alkoxy, and
   R₉ represents H, OH, lower alkyl (C₁-C₄) or lower alkoxy (C₁-C₄);
R₆ and R₇ are independently selected from H, CH₃, COOH, CONH₂, NH₂ or CH₂NH₂; and
R₄ represents hydrogen or C₁-C₁₅ saturated or unsaturated aliphatic hydrocarbon group, wherein
   1 to 6 hydrogen atoms of C₁-C₁₅ saturated or unsaturated aliphatic hydrocarbon group may be substituted with a group independently selected from halogen, arylalkyl, arylalkenyl, heteroarylalkyl or heteroarylalkenyl, wherein
      heteroarylalkyl or heteroarylalkenyl may have 1 to 2 heteroatoms independently selected nitrogen, oxygen or sulphur, and
      any 1 to 3 hydrogen atoms on the ring of arylalkyl, arylalkenyl, heteroarylalkenyl may be optionally substituted with lower alkyl (C₁-C₄), lower perhalo alkyl (C₁-C₄), cyano, hydroxyl, nitro, lower alkoxycarbonyl, halogen, lower alkoxy (C₁-C₄), lower perhaloalkoxy (C₁-C₄), unsubstituted amino, N-lower alkylamino (C₁-C₄), or N-lower alkylamino carbonyl (C₁-C₄);
         b. Formula II is:

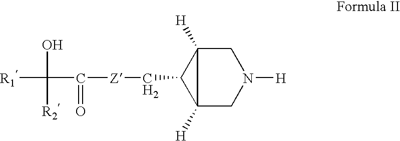

or a pharmaceutically acceptable salt, pharmaceutically acceptable solvate, ester, enantiomer, diastereomer, N-oxide, polymorph or metabolite thereof, wherein
R₁′ and R₂′ are independently selected from C₁-C₆ alkyl, C₃-C₇ cycloalkyl or phenyl, wherein phenyl is optionally substituted with one or more groups independently selected from C₁-C₃ alkyl, C₁-C₃ alkoxy or halogen; and
Z′ represents oxygen or NR₃, wherein
   R₃ represents hydrogen or C₁-C₃ alkyl;
      c. Formula III is,

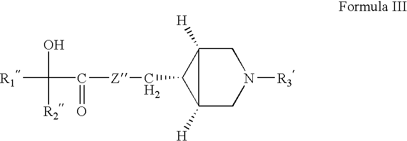

or a pharmaceutically acceptable salt, pharmaceutically acceptable solvate, ester, enantiomer, diastereomer, N-oxide, polymorph, prodrug or metabolite thereof, wherein
R₁″ and R₂″ are independently selected from C₁-C₆ alkyl, C₃-C₇ cycloalkyl, C₃-C₇ cycloalkenyl or phenyl, wherein phenyl is optionally substituted with one or more groups independently selected from C₁-C₃ alkyl, C₁-C₃ alkoxy or halogen;
R₃′ represents C₁-C₆ alkyl, wherein
   1-3 hydrogen atom(s) may be substituted with a group independently selected from C₅-C₇ cycloalkyl, 1,3-dioxo-1,3-dihydro-isoindolyl or phenyl, wherein phenyl is optionally substituted with one or more groups independently selected C₁-C₄ alkyl or halogen; and
Z represents oxygen or NR₄′, wherein
   R₄′ represents hydrogen or C₁-C₃ alkyl.

Pharmaceutical compositions described herein can include one or more of the following compounds of Formula I, II and Formula III, for example members of the list of such compounds presented herein:
(2R,2S)(1a,5a,6a)-N-(3-benzyl-3-azabicyclo[3.1.0]hex-6-yl-methyl)-2-hydroxy-2-methyl-(N-methyl)-2-phenylacetamide (Compound No. 148),
N-[(1α,5α,6α)-3-azabicyclo[3.1.0]hex-6-yl-methyl]-2-phenyl-2-hydroxy-2-(N-methyl)phenyl acetamide hydrochloride (Compound No. 149), or
Tartarate salt of (3-benzyl-3-azabicyclo[3.1.0]hex-6-yl)methyl cyclopentyl(hydroxy)-2-thienylacetate (Compound No. 150).

In another general aspect there is provided methods of treating or preventing autoimmune, inflammatory, or allergic diseases or disorders, which comprises administering to a mammal in need thereof a pharmaceutical composition comprising one or more muscarinic receptor antagonists (“MRA”) and at least one additional active ingredients selected from one or more β2-agonists, p38 MAP kinase inhibitors, PDE-IV inhibitors, corticosteroids or a mixture thereof and optionally one or more pharmaceutically acceptable carriers, excipients or diluents. Suitable MRA are one or more compounds having the structures of Formula I, II, or III as defined above.

### DETAILED DESCRIPTION OF THE INVENTION

In one aspect, there is provided pharmaceutical compositions comprising one or more muscarinic receptor antagonists (“MRA”) and at least one additional active ingredients selected from one or more β2-agonists, p38 MAP kinase inhibitors, PDE-IV inhibitors, corticosteroids or a mixture thereof and optionally one or more pharmaceutically acceptable carriers, excipients or diluents

MRA described herein include compounds having the structures of Formula I, II, or III, wherein

### Formula I is:

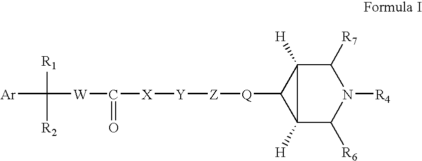

or a pharmaceutically acceptable salt, pharmaceutically acceptable solvate, ester, enantiomer, diastereomer, N-oxide, polymorphs, prodrugs or metabolite thereof, wherein
Ar represents an aryl or a heteroaryl ring having 1-2 heteroatoms independently selected from oxygen, sulphur or nitrogen, wherein
   the aryl or heteroaryl ring may be unsubstituted or substituted by one to three substituents independently selected from lower alkyl (C₁-C₄), lower perhalo alkyl (C₁-C₄), cyano, hydroxy, nitro, lower alkoxy (C₁-C₄), lower perhalo alkoxy (C₁-C₄), unsubstituted amino, N-lower alkyl (C₁-C₄), N-aryl amino, amino carbonyl, N-lower alkyl (C₁-C₄) or N-aryl amino carbonyl;
R₁ represents hydrogen, hydroxy, hydroxy methyl, substituted or unsubstituted amino, alkoxy, carbamoyl or halogen (e.g., fluorine, chlorine, bromine and iodine);
R₂ represents alkyl, (C₃-C₇) cycloalkyl ring, (C₃-C₇) cycloalkenyl ring, aryl, heterocyclic ring, or heteroaryl ring, wherein
   the heterocyclic ring or heteroaryl ring may have 1 to 2 heteroatoms independently selected from oxygen, sulphur or nitrogen, and
   the aryl or heteroaryl ring may be unsubstituted or substituted by one to three substituents independently selected from lower alkyl (C₁-C₄), lower perhalo alkyl (C₁-C₄), cyano, hydroxy, nitro, lower alkoxycarbonyl, halogen, lower alkoxy (C₁-C₄), lower perhalo alkoxy (C₁-C₄), unsubstituted amino, N-lower alkyl (C₁-C₄) or N-aryl amino, amino carbonyl, N-lower alkyl (C₁-C₄) or N-aryl amino carbonyl;
W represents (CH₂)ₚ, wherein p represents 0 to 1;
X represents oxygen, sulphur, —NR or no atom (i.e., a bond), wherein
   R represents hydrogen or (C₁₋₆) alkyl;
Y represents CHR₅CO or (CH₂)_{q}, wherein
   R₅ represents hydrogen or methyl, and
   q represents 0 to 4;
Z represents oxygen, sulphur, or NR₁₀, wherein
   R₁₀ represents hydrogen, or C₁₋₆ alkyl;
Q represents (CH₂)ₙ, CH₈ or CH₂CHR₉, wherein
   n represents 0 to 4,
   R₈ represents H, OH, C₁₋₆, alkyl, C₁₋₆ alkenyl, or C₁₋₆ alkoxy, and
   R₉ represents H, OH, lower alkyl (C₁-C₄) or lower alkoxy (C₁-C₄);
R₆ and R₇ are independently selected from H, CH₃, COOH, CONH₂, NH₂ or CH₂NH₂; and
R₄ represents hydrogen or C₁-C₁₅ saturated or unsaturated aliphatic hydrocarbon group, wherein
   1 to 6 hydrogen atoms of C₁-C₁₅ saturated or unsaturated aliphatic hydrocarbon group may be substituted with a group independently selected from halogen, arylalkyl, arylalkenyl, heteroarylalkyl or heteroarylalkenyl, wherein
      heteroarylalkyl or heteroarylalkenyl may have 1 to 2 heteroatoms independently selected nitrogen, oxygen or sulphur, and
      any 1 to 3 hydrogen atoms on the ring of arylalkyl, arylalkenyl, heteroarylalkenyl may be optionally substituted with lower alkyl (C₁-C₄), lower perhalo alkyl (C₁-C₄), cyano, hydroxyl, nitro, lower alkoxycarbonyl, halogen, lower alkoxy (C₁-C₄), lower perhaloalkoxy (C₁-C₄), unsubstituted amino, N-lower alkylamino (C₁-C₄), or N-lower alkylamino carbonyl (C₁-C₄);
         b. Formula II is:

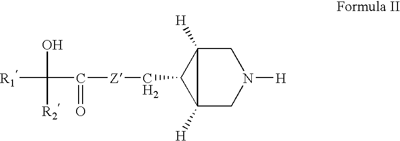

or a pharmaceutically acceptable salt, pharmaceutically acceptable solvate, ester, enantiomer, diastereomer, N-oxide, polymorph or metabolite thereof, wherein
R₁′ and R₂′ are independently selected from C₁-C₆ alkyl, C₃-C₇ cycloalkyl or phenyl, wherein phenyl is optionally substituted with one or more groups independently selected from C₁-C₃ alkyl, C₁-C₃ alkoxy or halogen; and
Z′ represents oxygen or NR₃, wherein
   R₃ represents hydrogen or C₁-C₃ alkyl;
      c. Formula III is,

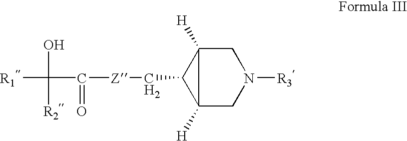

or a pharmaceutically acceptable salt, pharmaceutically acceptable solvate, ester, enantiomer, diastereomer, N-oxide, polymorph, prodrug or metabolite thereof, wherein
R₁″ and R₂″ are independently selected from C₁-C₆ alkyl, C₃-C₇ cycloalkyl, C₃-C₇ cycloalkenyl or phenyl, wherein phenyl is optionally substituted with one or more groups independently selected from C₁-C₃ alkyl, C₁-C₃ alkoxy or halogen;
R₃′ represents C₁-C₆ alkyl, wherein
   1-3 hydrogen atom(s) may be substituted with a group independently selected from C₅-C₇ cycloalkyl, 1,3-dioxo-1,3-dihydro-isoindolyl or phenyl, wherein
      phenyl is optionally substituted with one or more groups independently selected C₁-C₄ alkyl or halogen; and
Z represents oxygen or NR₄′, wherein
   R₄′ represents hydrogen or C₁-C₃ alkyl.

The pharmaceutical compositions of each of the above aspects can include one or more of the following embodiments. For example, the one or more compounds of Formula I, II and Formula III can be selected from;
(1a,5a,6a)-N-[3-benzyl-3-azabicyclo[3.1.0]hexyl-6-(aminomethyl)-yl]-2-hydroxy-2,2-diphenyl acetamide (Compound No. 1),
(1a,5a,6a)-N-[3-benzyl-3-azabicyclo[3.1.0]hexyl-6-(aminomethyl)-yl]-2-hydroxy-2-cyclohexyl-2-phenyl acetamide (Compound No. 2),
(1a,5a,6a)-N-[3-benzyl-3-azabicyclo[3. O]hexyl-6-(aminomethyl)-yl]-2-hydroxy-2-cyclopentyl-2-phenyl acetamide (Compound No. 3),
(1a,5a,6a)-N-[3-benzyl-3-azabicyclo[3.1.0]hexyl-6-(methyl)-yl]-2-hydroxy-2,2-diphenyl acetate (Compound No. 4),
(1a,5a,6a)-[3-benzyl-3-azabicyclo[3.1.0]hexyl-6-(methyl)-yl]-2-hydroxy-2-cyclohexyl-2-phenyl acetate (Compound No. 5),
(1a,5a,6a)-[3-benzyl-3-azabicyclo[3.1.0]hexyl-6-(methyl)-yl]-2-hydroxy-2-cyclopentyl-2-phenyl acetate (Compound No. 6),
(1a,5a,6a)-[3-(2-(2,3-dihydrobenzofuran-5-yl)ethyl)-3-azabicyclo[3.1.0]hexyl-6-(methyl)-yl]-2-hydroxy-2-cyclohexyl-2-phenyl acetate (Compound No. 7),
(1a,5a,6a)-[3-(2-(2,3-dihydrobenzofuran-5-yl)ethyl)-3-azabicyclo[3.1.0]hexyl-6-(methyl)-yl]-2-hydroxy-2-cyclopentyl-2-phenyl acetate (Compound No. 8),
(1a,5a,6a)-N-[3-(2-(2,3-dihydrobenzofuran-5-yl)ethyl)-3-azabicyclo[3.1.0]hexyl-6-(aminomethyl)-yl]-2-hydroxy-2-cyclohexyl-2-phenyl acetamide (Compound No. 9),
(1a,5a,6a)-N-[3-(2-(2,3-dihydrobenzofuran-5-yl)ethyl)-3-azabicyclo[3.1.0]hexyl-6-(aminomethyl)-yl]-2-hydroxy-2-cyclopentyl-2-phenyl acetamide (Compound No. 10),
(1a,5a,6a)-[3-(2-(3,4-methylenedioxyphenyl)ethyl)-3-azabicyclo[3.1.0]hexyl-6-(methyl)-yl]-2-hydroxy-2-cyclopentyl-2-phenyl acetate (Compound No. 11),
(1a,5a,6a)-[3-(2-(3,4-methylenedioxyphenyl)ethyl)-3-azabicyclo[3.1.0]hexyl-6-(methyl)-yl]-2-hydroxy-2-cyclohexyl-2-phenyl acetate (Compound No. 12),
(1a,5a,6a)-N-[3-(2-(3,4-methylenedioxyphenyl)ethyl)-3-azabicyclo[3.1.0]hexyl-6-(aminomethyl)-yl]-2-hydroxy-2-cyclopentyl-2-phenyl acetamide (Compound No. 13),
(1a,5a,6a)-N-[3-(2-(3,4-methylenedioxyphenyl)ethyl)-3-azabicyclo[3.1.0]hexyl-6-(aminomethyl)-yl]-2-hydroxy-2-cyclohexyl-2-phenyl acetamide (Compound No. 14),
(1a,5a,6a)-N-[3-(4-methyl-3-pentenyl)-3-azabicyclo[3.1.0]hexyl-6-(aminomethyl)-yl]-2-hydroxy-2-cyclohexyl-2-phenyl acetamide (Compound No. 15),
(1a,5a,6a)-N-[3-(4-methyl-3-pentenyl)-3-azabicyclo[3.1.0]hexyl-6-(aminomethyl)-yl]-2-hydroxy-2-cyclopentyl-2-phenyl acetamide (Compound No. 16),
(1a,5a,6a)-[3-(4-methyl-3-pentenyl)-3-azabicyclo[3.1.0]hexyl-6-(methyl)-yl]-2-hydroxy-2-cyclohexyl-2-phenyl acetate (Compound No. 17),
(1a,5a,6a)-[3-(4-methyl-3-pentenyl)-3-azabicyclo[3.1.0]hexyl-6-(methyl)-yl]-2-hydroxy-2-cyclopentyl-2-phenyl acetate (Compound No. 18),
(1a,5a,6a)-[3-(1-phenylethyl)-3-azabicyclo[3.1.0]hexyl-6-(methyl)-yl]-2-hydroxy-2-cyclopentyl-2-phenyl acetate (Compound No. 19),
(1a,5a,6a)-[3-(1-phenylethyl)-3-azabicyclo[3.1.0]hexyl-6-(methyl)-yl]-2-hydroxy-2-cyclohexyl-2-phenyl acetate (Compound No. 20),
(1a,5a,6a)-N-[3-(1-phenylethyl)-3-azabicyclo[3.1.0]hexyl-6-(aminomethyl)-yl]-2-hydroxy-2-cyclohexyl-2-phenyl acetamide (Compound No. 21),
(1a,5a,6a)-N-[3-(1-phenylethyl)-3-azabicyclo[3.1.0]hexyl-6-(aminomethyl)-yl]-2-hydroxy-2-cyclopentyl-2-phenyl acetamide (Compound No. 22),
(1a,5a,6a)-N-[3-benzyl-3-azabicyclo[3.1.0]hexyl-6-(1-aminoethyl)-yl]-2-hydroxy-2,2-diphenyl acetamide (Compound No. 23),
(1a,5a,6a)-N-[3-benzyl-3-azabicyclo[3.0]hexyl-6-(1-aminoethyl)-yl]-2-hydroxy-2-cyclohexyl-2-phenyl acetamide (Compound No. 24),
(1a,5a,6a)-N-[3-benzyl-3-azabicyclo[3.1.0]hexyl-6-(1-aminoethyl)-yl]-2-hydroxy-2-cyclopentyl-2-phenyl acetamide (Compound No. 25),
(1a,5a,6a)-[3-(3-methyl-2-butenyl)-3-azabicyclo[3.1.0]hexyl-6-(methyl)-yl]-2-hydroxy-2-cyclohexyl-2-phenyl acetate (Compound No. 26),
(1a,5a,6a)-[3-(3-methyl-2-butenyl)-3-azabicyclo[3.1.0]hexyl-6-(methyl)-yl]-2-hydroxy-2-cyclopentyl-2-phenyl acetate (Compound No. 27),
(2R)-(+)-(1a,5a,6a)-N-[3-benzyl-3-azabicyclo[3.1.0]hexyl-6-(aminomethyl)-yl]-2-hydroxy-2-cyclohexyl-2-phenyl acetamide (Compound No. 28),
(2R)-(+)-(1a,5a,6a)-N-[3-benzyl-3-azabicyclo[3.1.0]hexyl-6-(aminomethyl)-yl]-2-hydroxy-2-cyclopentyl-2-phenyl acetamide (Compound No. 29),
(2R)(+)-(1a,5a,6a)-[3-benzyl-3-azabicyclo[3.1.0]hexyl-6-(methyl)-yl]-2-hydroxy-2-cyclohexyl-2-phenyl acetate (Compound No. 30),
(2R)(+)-(1a,5a,6a)-[3-benzyl-3-azabicyclo[3.1.0]hexyl-6-(methyl)-yl]-2-hydroxy-2-cyclopentyl-2-phenyl acetate (Compound No. 31),
(2S)-(−)-(1a,5a,6a)-N-[3-benzyl-3-azabicyclo[3.1.0]hexyl-6-(aminomethyl)-yl]-2-hydroxy-2-cyclopentyl-2-phenyl acetamide (Compound No. 32),
(2S)-(−)-(1a,5a,6a)-[3-benzyl-3-azabicyclo[3.1.0]hexyl-6-(methyl)-yl]-2-hydroxy-2-cyclopentyl-2-phenyl acetate (Compound No. 33),
(1a,5a,6a)-N-[3-benzyl-3-azabicyclo[3.1.0]hexyl-6-(aminomethyl)-yl]-2-hydroxy-2-cyclopentyl-2-phenyl acetamide L-(+)-tartrate salt (Compound No. 34),
(2R)-(+)-(1a,5a,6a)-N-[3-benzyl-3-azabicyclo[3.1.0]hexyl-6-(aminomethyl)-yl]-2-hydroxy-2-cyclohexyl-2-phenyl acetamide. L-(+)-tartrate salt (Compound No. 35),
(2R)-(+)-(1a,5a,6a)-N-[3-benzyl-3-azabicyclo[3.1.0]hexyl-6-(aminomethyl)-yl]-2-hydroxy-2-cyclopentyl-2-phenyl acetamide. L-(+)-tartrate salt (Compound No. 36),
(1a,5a,6a)-N-[3-benzyl-3-azabicyclo[3.1.0]hexyl-6-(aminomethyl)-yl]-2-hydroxy-2-cyclobutyl-2-phenyl acetamide (Compound No. 37),
(1a,5a,6a)-N-[3-benzyl-3-azabicyclo[3.1.0]hexyl-6-(aminomethyl)-yl]-2-hydroxy-2-cyclopropyl-2-phenyl acetamide (Compound No. 38),
(1a,5a,6a)-N-[3-(3-methyl-2-butenyl)-3-azabicyclo[3.1.0]hexyl-6-(aminomethyl)-yl]-2-hydroxy-2-cyclohexyl-2-phenyl acetamide (Compound No. 39),
(1a,5a,6a)-[3-(3,4-methylenedioxyphenyl)methyl-3-azabicyclo[3.1.0]hexyl-6-(methyl)-yl]-2-hydroxy-2-cyclopentyl-2-phenyl acetate (Compound No. 40),
(1a,5a,6a)-[3-(2-(3,4-methylenedioxyphenyl)ethyl)-3-azabicyclo[3.1.0]hexyl-6-(methyl)-yl]-2-hydroxy-2-cyclopentyl-2-phenyl acetate. L-(+)-tartrate salt (Compound No. 41),
(1a,5a,6a)-[3-benzyl-3-azabicyclo[3.1.0]hexyl-6-(methyl)-yl]-2-hydroxy-2,2 diphenyl acetate L(+)-tartrate salt (Compound No. 42),
(1a,5a,6a)-[3-benzyl-3-azabicyclo[3.0]hexyl-6-(methyl)-yl]-2-hydroxy-2-cyclohexyl-2-phenyl acetate L(+)-tartrate salt (Compound No. 43),
(1a5a,6a)-[3-benzyl-3-azabicyclo[3.1.0]hexyl-6-(methyl)-yl]-2-hydroxy-2-cyclopentyl-2-phenyl acetate L(+)-tartrate salt (Compound No. 44),
(1a,5a,6a)-N-[3-(3-pyridylmethyl)-3-azabicyclo[3.1.0]hexyl-6-(aminomethyl)-yl]-2-hydroxy-2-cyclohexyl-2-phenyl acetamide (Compound No. 45),
(1a,5a,6a)-N-[3-(4-pyridylmethyl)-3-azabicyclo[3.1.0]hexyl-6-(aminomethyl)-yl]-2-hydroxy-2-cyclohexyl-2-phenyl acetamide (Compound No. 46),
(1a,5a,6a)-N-[3-(2-pyridylmethyl)-3-azabicyclo[3.1.0]hexyl-6-(aminomethyl)-yl]-2-hydroxy-2-cyclohexyl-2-phenyl acetamide (Compound No. 47),
(1a,5a,6a)-N-[3-(4-pyridylmethyl)-3-azabicyclo[3.1.0]hexyl-6-(aminomethyl)-yl]-2-hydroxy-2-cyclopentyl-2-phenyl acetamide (Compound No. 48),
(1a,5a,6a)-N-[3-(3-pyridylmethyl)-3-azabicyclo[3.1.0]hexyl-6-(aminomethyl)-yl]-2-hydroxy-2,2-diphenyl acetamide (Compound No. 49),
(1a,5a,6a)-N-[3-(4-pyridylmethyl)-3-azabicyclo[3.1.0]hexyl-6-(aminomethyl)-yl]-2-hydroxy-2,2-diphenyl acetamide (Compound No. 50),
(1a,5a,6a)-N-[3-(2-pyridylmethyl)-3-azabicyclo[3. O]hexyl-6-(aminomethyl)-yl]-2-hydroxy-2,2-diphenyl acetamide (Compound No. 51),
(1a,5a,6a)-N-[3-(2-pyridylmethyl)-3-azabicyclo[3.1.0]hexyl-6-(aminomethyl)-yl]-2-hydroxy-2-cyclopentyl-2-phenyl acetamide (Compound No. 52),
(1a,5a,6a)-N-[3-(3-pyridylmethyl)-3-azabicyclo[3.1.0]hexyl-6-(aminomethyl)-yl]-2-hydroxy-2-cyclopentyl-2-phenyl acetamide (Compound No. 53),
(1a,5a,6a)-N-[3-(3-methyl-2-butenyl)-3-azabicyclo[3.0]hexyl-6-(aminomethyl)-yl]-2-hydroxy-2-cyclopentyl-2-phenyl acetamide (Compound No. 54),
(1a,5a,6a)-N-[3-(3,4-methylenedioxyphenyl)methyl-3-azabicyclo[3.1.0]hexyl-6-(aminomethyl)-yl]-2-hydroxy-2-cyclopentyl-2-phenyl acetamide (Compound No. 55),
(1a,5a,6a)-N-[3-(3,4-methylenedioxyphenyl)methyl-3-azabicyclo[3.1.0]hexyl-6-(aminomethyl)-yl]-2-hydroxy-2-cyclohexyl-2-phenyl acetamide (Compound No. 56),
(1a,5a,6a)-[3-(4-methyl-3-pentenyl)-3-azabicyclo[3.1.0]hexyl-6-(methyl)-yl]-2-hydroxy-2-cyclohexyl-2-phenyl acetate. L(+) tartrate salt (Compound No. 57),
(1a,5a,6a)-[3-(2-(3,4-methylenedioxyphenyl)ethyl)-3-azabicyclo[3.1.0]hexyl-6-(methyl)-yl]-2-hydroxy-2-cyclohexyl-2-phenyl acetate. L(+) tartrate salt (Compound No. 58),
(1a,5a,6a)-[3-(1-phenylethyl)-3-azabicyclo[3.1.0]hexyl-6-(methyl)-yl]-2-hydroxy-2-cyclopentyl-2-phenyl acetate. L(+) tartrate salt (Compound No. 59),
(1a,5a,6a)-N-[3-benzyl-3-azabicyclo[3.1.0]-hexyl-6-(aminomethyl)-yl]-2-hydroxy-2-cyclopentyl-2-phenyl acetamide hydrochloride salt (Compound No. 60),
(1a,5a,6a)-N-[3-benzyl-3-azabicyclo[3.1.0]-hexyl-6-(aminomethyl)-yl]-2-hydroxy-2-cyclopentyl-2-phenyl acetamide. L(−) malic acid salt (Compound No. 61),
(1a,5a,6a)-N-[3-benzyl-3-azabicyclo[3.1.0]-hexyl-6-(aminomethyl)-yl]-2-hydroxy-2-cyclopentyl-2-phenyl acetamide, maleate salt (Compound No. 62),
(2R,2S)(1a,5a,6a)-N-[3-azabicyclo[3.11.]hexyl-6-(aminomethyl)-yl]-2-hydroxy-2-cyclopentyl-2-phenyl acetamide (Compound No. 63),
(2R,2S)(1a,5a,6a)-N-[3-azabicyclo[3.1.0]hexyl-6-(aminomethyl)-yl]-2-hydroxy-2-cyclopentyl-2-phenyl acetamide hydrochloride salt (Compound No. 64),
(2R)-(1a,5a,6a)-N-[3-azabicyclo[3.1.0]hexyl-6-(aminomethyl)-yl]-2-hydroxy-2-cyclopentyl 2-phenyl acetamide (Compound No. 65),
(2R)-(1a,5a,6a)-N-[3-azabicyclo[3.0]hexyl-6-(aminomethyl)-yl]-2-hydroxy-2-cyclopentyl 2-phenyl acetamide hydrochloride salt (Compound No. 66),
(2S)-(1a,5a,6a)-N-[3-azabicyclo[3.1.0]hexyl-6-(aminomethyl)-yl]-2-hydroxy-2-cyclopentyl 2-phenyl acetamide (Compound No. 67),
(2S)-(1a,5a,6a)-N-[3-azabicyclo[3.1.0]hexyl-6-(aminomethyl)-yl]-2-hydroxy-2-cyclopentyl 2-phenyl acetamide hydrochloride salt (Compound No. 68),
(2R,2S)(1a,5a,6a)-N-[3-azabicyclo[3.1.0]hexyl-6-(aminomethyl)-yl]-2-methoxy-2-cyclopentyl-2-phenyl acetamide (Compound No. 69),
(2R,2S)(1a,5a,6a)-N-[3-azabicyclo[3.1.0]hexyl-6-(aminomethyl)-yl]-2-hydroxy-2-cycloheptyl-2-phenyl acetamide (Compound No. 70),
(2R,2S)(1a,5a,6a)-N-[3-azabicyclo[3.1.0]hexyl-6-(aminomethyl)-yl]-2-hydroxy-2-cyclobutyl-2-phenyl acetamide (Compound No. 71),
(2R,2S)(1a,5a,6a)-N-[3-azabicyclo[3.1.0]hexyl-6-(aminomethyl)-yl]-2-hydroxy-2-cyclobutyl-2-phenyl acetamide tartarate salt (Compound No. 72),
(2R)(1a,5a,6a)-N-[3-azabicyclo[3.1.0]hexyl-6-(aminomethyl)-yl]-2-hydroxy-2-(3,3-difluorocyclopentyl)-2-phenyl acetamide (Compound No. 73),
(2R,2S)(1a,5a,6a)-N-[3-azabicyclo[3.1.0]hexyl-6-(aminomethyl)-yl]-2-hydroxy-2-(3-fluorocyclopentyl)-2-phenyl acetamide (Compound No. 74),
(2R,2S)(1a,5a,6a)-N-[3-azabicyclo[3,0]hexyl-6-(aminomethyl)-yl]-2-hydroxy-2-(3,3-difluorocyclopentyl)-2-phenyl acetamide (Compound No. 75),
(2R,2S)(1a,5a,6a)-N-[3-azabicyclo[3.1.0]hexyl-6-(aminomethyl)-yl]-2-hydroxy-2-(3,3-difluorocyclopentyl)-2-phenyl acetamide tartarate salt (Compound No. 76),
(2R,2S)(1a,5a,6a)-N-[3-azabicyclo[3.1.0]hexyl-6-(aminomethyl)-yl]-2-hydroxy-2,2-diphenyl acetate (Compound No. 77),
(2R,2S)(1a,5a,6a)-N-[3-azabicyclo[3.1.0]hexyl-6-(aminomethyl)-yl]-2-hydroxy-2,2-diphenyl acetamide (Compound No. 78),
(2R,2S)(1a,5a,6a)-N-[3-azabicyclo[3.1.0]hexyl-6-(aminomethyl)-yl]-2-hydroxy-2-cyclohexyl-2-phenyl acetamide (Compound No. 79),
(2R,2S)(1a,5a,6a)-N-[3-azabicyclo[3.1.0]hex-6-ylmethyl)-2-cyclopentyl-2-hydroxy-N-methyl-2-phenyl acetamide (Compound No. 80),
N-[(1α,5α,6α)-3-azabicyclo[3.1.0]hex-6-yl-methyl]-2-phenyl-2-hydroxy-2-(N-methyl)phenylacetamide (Compound No. 81),
N-[(1α,5α,6α)-3-azabicyclo[3.1.0]hex-6-yl-methyl]-2-phenyl-2-hydroxy-2-(N-methyl)phenylacetamide tartarate salt (Compound No. 82),
(2R,2S)—N-[(1α,5α,6α)-3-azabicyclo[3.1.0]hex-6-yl-methyl]-2-isopropyl-2-hydroxy-2-phenylacetamide (Compound No. 83),
(2R,2S)—N-[(1α,5α,6α)-3-azabicyclo[3.1.0]hex-6-yl-methyl]-2-isopropyl-2-hydroxy-2-phenylacetamide hydrochloride salt (Compound No. 84),
(2R,2S)—N-[(1α,5α,6α)-3-azabicyclo[3.1.0]hex-6-yl-methyl]-2-(3-pentyl)-2-hydroxy-2-phenyl acetamide (Compound No. 85),
(2R,2S)-[(1α,5α,6α)-3-azabicyclo[3.1.0]hex-6-yl-methyl]-2-cyclopentyl-2-hydroxy-2-phenyl acetic acid (Compound No. 86),
(2R)—N-[(1α,5α,6α)-3-azabicyclo[3.1.0]hex-6-yl-methyl]-2-cyclopentyl-2-hydroxy-2-(N-methyl)phenylacetamide (Compound No. 87),
(2R)—N-[(1α,5α,6α)-3-azabicyclo[3.1.0]hex-6-yl-methyl]-2-cyclopentyl-2-hydroxy-2-(N-methyl)phenylacetamide hydrochloride salt (Compound No. 88),
(2R,2S)-[(1α,5α,6α)-3-azabicyclo[3.1.0]hex-6-yl-methyl]-2-methyl-2-hydroxy-2-phenylacetic acid ester (Compound No. 89),
(2R,2S)-[(1α,5α,6α)-3-azabicyclo[3.1.0]hex-6-yl-methyl]-2-isopropyl-2-hydroxy-2-phenylacetic acid ester (Compound No. 90),
(2R,2S)-[(1α,5α,6α)-3-azabicyclo[3.1.0]hex-6-yl-methyl]-2-(3-pentyl)-2-hydroxy-2-phenylacetic acid ester (Compound No. 91),
(2R,2S)—N-[(1α,5α,6α)-3-azabicyclo[3.1.0]hex-6-yl-methyl]-2-methyl-2-hydroxy-2-phenylacetamide (Compound No 92),
(2R)—N-[(1α,5α,6α)-3-azabicyclo[3.1.0]hex-6-yl-methyl]-2-isopropyl-2-hydroxy-2-(N-methyl)phenylacetamide (Compound No. 93),
(2R,2S)-[(1α,5α,6α)-3-azabicyclo[3.1.0]hex-6-yl-methyl]-2-(m-methylphenyl)-2-hydroxy-2-phenylacetic acid ester (Compound No. 94),
(2R,2S)—N-[(1α,5α,6α)-3-azabicyclo[3.1.0]hex-6-yl-methyl]-2-(p-fluorophenyl)-2-hydroxy-2-phenylacetamide (Compound No. 95),
(2R,2S)—N-[(1α,5α,6α)-3-azabicyclo[3.1.0]hex-6-yl-methyl]-2-(p-methylphenyl)-2-hydroxy-2-phenylacetamide (Compound No. 96),
(2R)—N-[(1α,5α,6α)-3-azabicyclo[3.1.0]hex-6-yl-methyl]-2-(p-fluorophenyl)-2-hydroxy-2-(N-methyl)phenylacetamide (Compound No. 97),
(2R)—N-[(1α,5α,6α)-3-azabicyclo[3.1.0]hex-6-yl-methyl]-2-p-methylphenyl)-2-hydroxy-2-(N-methyl)phenylacetamide (Compound No. 98),
(2R,2S)(1a,5a,6a)-N-{-[4-(1,3-dioxo-1,3-dihydro-isoindol-2-yl)-butyl]-3-azabicyclo [3.1.0]hex-6-yl-methyl}-2-hydroxy-2-cyclopentyl-2-phenylacetamide (Compound No. 99),
(2R)(1a,5a,6a)-N-(3-Benzyl-3-azabicyclo[3.1.0]hex-6-yl-methyl)-2-hydroxy-2-cyclopent-1-enyl-2-phenylacetamide (Compound No. 100),
(2R,2S)(1a,5a,6a)-N-(3-Isopropyl-3-azabicyclo[3.1.0]hex-6-yl-methyl)-2-hydroxy-2-cyclopentyl-2-phenylacetamide (Compound No. 101),
(2R,2S)(1a,5a,6a)-N-(3-Diphenylmethyl-3-azabicyclo[3.1.0]hex-6-yl-methyl)-2-hydroxy-2-cyclopentyl-2-phenylacetamide (Compound No. 102),
(2R,2S)(1a,5a,6a)-N-(3-sec-butyl-3-azabicyclo[3.1.0]hex-6-yl-methyl)-2-hydroxy-2-cyclopentyl-2-phenylacetamide (Compound No. 103),
(2R,2S)(1a,5a,6a)-N-(3-Benzyl-3-azabicyclo[3.1.0]hex-6-yl-methyl)-2-hydroxy-2-(3-pentyl)-2-phenylacetamide (Compound No. 104),
(2R,2S)(1a,5a,6a)-N-(3-Benzyl-3-azabicyclo[3.1.0]hex-6-yl-methyl)-2-hydroxy-2-cyclohexyl-2-(4-methoxyphenyl)acetamide (Compound No. 105),
(1a,5a,6a)-N-(3-Benzyl-3-azabicyclo[3.1.0]hex-6-yl-methyl)-2-hydroxy-2-phenyl-(N-ethyl)-2-phenylacetamide (Compound No. 106),
(2R,2S)(1a,5a,6a)-N-(3-Benzyl-3-azabicyclo[3.1.0]hex-6-yl-methyl)-2-hydroxy-2-cyclopentyl-(N-ethyl)-2-phenylacetamide (Compound No. 107),
(2R,2S)(1a,5a,6a)-N-(3-Benzyl-3-azabicyclo[3.1.0]hex-6-yl-methyl)-2-hydroxy-2-cyclohexyl-(N-ethyl)-2-phenylacetamide (Compound No. 108),
(2R,2S)(1a,5a,6a)-N-(3-Benzyl-3-azabicyclo[3.1.0]hex-6-yl-methyl)-2-hydroxy-2-(3-pentyl)-(N-methyl)-2-phenylacetamide (Compound No. 109),
(2R,2S)(1a,5a,6a)-N-(3-Benzyl-3-azabicyclo[3.1.0]hex-6-yl-methyl)-2-hydroxy-2-(sec-butyl)-(N-methyl)-2-phenylacetamide (Compound No. 110),
(2R,2S)(1a,5a,6a)-N-(3-Benzyl-3-azabicyclo[3.1.0]hex-6-yl-methyl)-2-hydroxy-2-isopropyl-(N-methyl)-2-phenylacetamide (Compound No. 111),
(2R,2S)(1a,5a,6a)-N-[3-(4-tert-butyl-benzyl)-3-azabicyclo[3.1.0]hex-6-yl-methyl]-2-hydroxy-2-cyclopentyl-2-phenylacetamide (Compound No. 112),
(2R,2S)(1a,5a,6a)-N-(3-Benzyl-3-azabicyclo[3.1.00]hex-6-yl-methyl)-2-hydroxy-2-cyclohex-2-enyl-2-phenylacetamide (Compound No. 113),
(1a,5a,6a)-N-[3-(4-methylbenzyl)-3-azabicyclo[3.1.0]hex-6-yl-methyl]-2-hydroxy-2,2-diphenylacetamide (Compound No. 114),
(2R,2S)(1a,5a,6a)-N-[3-(4-methylbenzyl)-3-azabicyclo[3.1.0]hex-6-yl-methyl]-2-hydroxy-2-cyclopentyl-2-phenylacetamide (Compound No. 115),
(2R,2S)(1a,5a,6a)-N-[3-(4-methylbenzyl)-3-azabicyclo[3.1.0]hex-6-yl-methyl]-2-hydroxy-2-cyclohexyl-2-phenylacetamide (Compound No. 116),
(1a,5a,6a)-N-[3-(3-methylbenzyl)-3-azabicyclo[3.1.0]hex-6-yl-methyl]-2-hydroxy-2,2-diphenylacetamide (Compound No. 117),
(1a,5a,6a)-N-[3-(3-fluorobenzyl)-3-azabicyclo[3.1.0]hex-6-yl-methyl]-2-hydroxy-2,2-diphenylacetamide (Compound No. 118),
(2R,2S)(1a,5a,6a)-N-[3-(3-fluorobenzyl)-3-azabicyclo[3.1.0]hex-6-yl-methyl]-2-hydroxy-2-cyclohexyl-2-phenylacetamide (Compound No. 119),
(2R,2S)(1a, 5a,6a)-N-[2-(2,4-difluorobenzyl)-3-azabicyclo[3.1.0]hex-6-yl-methyl]-2-hydroxy-2-cyclohexyl-2-phenylacetamide (Compound No. 120),
(1a,5a,6a)-N-[3-(2,4-difluorobenzyl)-3-azabicyclo[3.1.0]hex-6-yl-methyl]-2-hydroxy-2,2-diphenylacetamide (Compound No. 121),
(2R,2S)(1a,5a,6a)-N-[3-(3-methylbenzyl)-3-azabicyclo[3.1.0]hex-6-yl-methyl]-2-hydroxy-2-cyclopentyl-2-phenylacetamide (Compound No. 122),
(2R,2S)(1a,5a,6a)-N-(3-benzyl-3-azabicyclo[3.1.0]hex-6-yl-methyl)-2-hydroxy-2-(4-methylphenyl)-2-phenylacetamide (Compound No. 123),
(2R,2S)(1a,5a,6a)-N-(3-benzyl-3-azabicyclo[3.1.0]hex-6-yl-methyl)-2-hydroxy-2-(4-methylphenyl)-(N-methyl)-2-phenylacetamide (Compound No. 124),
(2R,2S)(1a,5a,6a)-N-(3-benzyl-3-azabicyclo[3.1.0]hex-6-yl-methyl)-2-hydroxy-2-(4-fluorophenyl)-2-phenylacetamide (Compound No. 125),
(2R,2S)(1a,5a,6a)-(3-benzyl-3-azabicyclo[3.1.0]hex-6-yl-methyl)-2-hydroxy-2-(4-fluorophenyl)-2-phenyl acetic acid ester (Compound No. 126),
(2R,2S)(1a,5a,6a)-N-(3-benzyl-3-azabicyclo[3.1.0]hex-6-yl-methyl)-2-hydroxy-2-(4-fluorophenyl)-(N-4-methyl)-2-phenylacetamide (Compound No. 127),
(2R,2S)(1a,5a,6a)-N-(3-benzyl-3-azabicyclo[3.1.0]hex-6-yl-methyl)-2-hydroxy-2-(3-methylphenyl)-2-phenylacetamide (Compound No. 128),
(2R,2S)(1a,5a,6a)-N-(3-benzyl-3-azabicyclo[3.1.0]hex-6-yl-methyl)-2-hydroxy-2-(3-methylphenyl)-(N-methyl)-2-phenylacetamide (Compound No. 129),
(2R,2S)(1a,5a,6a)-(3-benzyl-3-azabicyclo[3.1.0]hex-6-yl-methyl)-2-hydroxy-2-(3-methylphenyl)-2-phenyl acetic acid ester (Compound No. 130),
(2R,2S)(1a,5a,6a)-(3-benzyl-3-azabicyclo[3.1.0]hex-6-yl-methyl)-2-hydroxy-2-cyclopentyl-2-(3-methylphenyl)acetic acid ester (Compound No. 131),
(2R,2S)(1a,5a,6a)-(3-benzyl-3-azabicyclo[3.1.0]hex-6-yl-methyl)-2-hydroxy-2-cyclopentyl-2-(3-methylphenyl)acetic acid ester tartarate salt (Compound No. 132),
(2R,2S)(1a,5a,6a)-N-(3-benzyl-3-azabicyclo[3.1.0]hex-6-yl-methyl)-2-hydroxy-2-cyclopentyl-2-(3-methylphenyl)acetamide (Compound No. 133),
(2R,2S)(1a,5a,6a)-N-(3-benzyl-3-azabicyclo[3.1.0]hex-6-yl-methyl)-2-hydroxy-2-cyclopentyl-2-(3-methylphenyl)acetamide tartarate salt (Compound No. 134),
(1a,5a,6a)-N-(3-benzyl-3-azabicyclo[3.1.0]hex-6-yl-methyl)-2-hydroxy-2,2-di(4-fluorophenyl)acetic acid ester (Compound No. 135),
(1a,5a,6a)-N-(3-benzyl-3-azabicyclo[3.1.0]hex-6-yl-methyl]-2-hydroxy-2,2-di(4-fluorophenyl)-acetamide (Compound No. 136),
(2R,2S)(1a,5a,6a)-(3-benzyl-3-azabicyclo[3.1.0]hex-6-yl-methyl]-2-hydroxy-2-cyclobutyl-2-phenyl acetic acid ester (Compound No. 137),
(2R,2S)(1a,5a,6a)-N-(3-cyclohexylmethyl-3-azabicyclo[3.1.01]hex-6-yl-methyl)-2-hydroxy-2-cyclopentyl-2-phenylacetamide (Compound No. 138),
(2R)(1a,5a,6a)-N-(3-benzyl-3-azabicyclo[3.1.0]hex-6-yl-methyl)-2-hydroxy-2-cyclopentyl-(N-methyl)-2-phenylacetamide (Compound No. 139),
(2R,2S)(1a,5a,6a)-N-(3-benzyl-3-azabicyclo[3.1.0]hex-6-yl-methyl]-2-hydroxy-2-cyclopentyl-2-(4-methylphenyl)acetamide (Compound No. 140),
(2R,2S)(1a,5a,6a)-(3-benzyl-3-azabicyclo[3.1.0]hex-6-yl-methyl)-2-hydroxy-2-phenyl-2-(4-methylphenyl)acetic acid ester (Compound No. 141),
(2R,2S)(1a,5a,6a)-(3-benzyl-3-azabicyclo[3.1.0]hex-6-yl-methyl)-2-hydroxy-2-methyl-2-phenyl acetic acid ester (Compound No. 142),
(2R,2S)(1a,5a,6a)-N-(3-benzyl-3-azabicyclo[3.1.0]hex-6-yl-methyl]-2-hydroxy-2-methyl-2-phenyl acetamide (Compound No. 143),
(2R,2S)(1a,5a,6a)-(3-benzyl-3-azabicyclo[3.1.0]hex-6-yl-methyl)-2-hydroxy-2-isopropyl-2-phenyl acetic acid ester (Compound No. 144),
(1a,5a,6a)-N-(3-methyl-3-azabicyclo[3.1.0]hex-6-yl-methyl]-2-hydroxy-2-phenyl-(N-methyl)-2-phenylacetamide (Compound No. 145),
(1a,5a,6a)-N-(3-benzyl-3-azabicyclo[3.1.0]hex-6-yl-methyl]-2-hydroxy-2,2-di (3-methylphenyl)acetamide (Compound No. 146),
(2R,2S)(1a,5a,6a)-(3-benzyl-3-azabicyclo[3.1.0]hex-6-yl-methyl]-2-hydroxy-2-(3-pentyl)-2-phenyl acetic acid ester (Compound No. 147),
(2R,2S)(1a,5a,6a)-N-(3-benzyl-3-azabicyclo[3.1.0]hex-6-yl-methyl)-2-hydroxy-2-methyl-(N-methyl)-2-phenylacetamide (Compound No. 148),
N-[(1α,5α,6α)-3-azabicyclo[3.1.0]hex-6-yl-methyl]-2-phenyl-2-hydroxy-2-(N-methyl)phenyl acetamide hydrochloride (Compound No. 149), or
Tartarate salt of (3-benzyl-3-azabicyclo[3.1.0]hex-6-yl)methyl cyclopentyl(hydroxy)-2-thienylacetate (Compound No. 150).

Also provided are pharmaceutical dosage forms comprising a therapeutically effective amount of one or more compounds of Formula I, II, or III described herein, a therapeutically effective amount of one or more β2-agonists, and one or more pharmaceutically acceptable caters, excipients or diluents. Such pharmaceutical dosage form may also include a therapeutically effective amount of one or more corticosteroids, one or more p38 MAP kinase inhibitors, one or more PDE-IV inhibitors or combinations thereof.

Also provided are pharmaceutical dosage forms comprising a therapeutically effective amount of one or more compounds of Formula I, II or II described herein, a therapeutically effective amount of one or more corticosteroids, and one or more pharmaceutically acceptable carriers, excipients or diluents. Such pharmaceutical dosage form may also include a therapeutically effective amount of one or more β2-agonists, one or more p38 MAP kinase inhibitors, one or more PDE-IV inhibitors or combinations thereof.

Also provided are pharmaceutical dosage forms comprising a therapeutically effective amount of one or more compounds of Formula I, II, or III described herein, a therapeutically effective amount of one or more p38 MAP kinase inhibitors, and one or more pharmaceutically acceptable carriers, excipients or diluents. Such pharmaceutical dosage form may also include a therapeutically effective amount of one or more corticosteroids, one or more β2-agonists, one or more PDE-IV inhibitors or combinations thereof.

Also provided are pharmaceutical dosage forms comprising a therapeutically effective amount of one or more compounds of Formula I, II, or III described herein, a therapeutically effective amount of one or more PDE-IV inhibitors, and one or more pharmaceutically acceptable carriers, excipients or diluents. Such pharmaceutical dosage form may also include a therapeutically effective amount of one or more corticosteroids, one or more 132-agonists, one or more p38 MAP kinase inhibitors or combinations thereof.

Suitable β2-agonists as described herein may be any β2-agonist described in the art or subsequently discovered. For example, β2-agonists may include, but are not limited to, one or more compounds described in U.S. Pat. Nos. 3,705,233; 3,644,353; 3,642,896; 3,700,681; 4,579,985; 3,994,974; 3,937,838; 4,419,364; 5,126,375; 5,243,076; 4,992,474; and 4,011,258, each of which are incorporated herein by reference.

Examples of suitable β2-agonists include one or more of albuterol, salbutamol, biltolterol, pirbuterol, levosalbutamol, tulobuterol, terbutaline, bambuterol, metaproterenol, fenoterotl salmeterol, carmoterol, arformoterol, formoterol, and their pharmaceutically acceptable salts or solvates thereof or mixtures thereof.

Suitable corticosteroids as described herein may be any corticosteroid described in the art or subsequently discovered. For example, corticosteroids may include, but are not limited to, one or more compounds described in U.S. Pat. Nos. 3,312,590; 3,983,233; 3,929,768; 3,721,687; 3,436,389; 3,506,694; 3,639,434; 3,992,534; 3,928,326; 3,980,778; 3,780,177; 3,652,554; 3,947,478; 4,076,708; 4,124,707; 4,158,055; 4,298,604; 4,335,121; 4,081,541; 4,226,862; 4,290,962; 4,587,236; 4,472,392; 4,472,393; 4,242,334; 4,014,909; 4,098,803; 4,619,921; 5,482,934; 5,837,699; 5,889,015; 5,278,156; 5,015,746; 5,976,573; 6,337,324; 6,057,307; 6,723,713; 6,127,353; and 6,180,781, each of which are incorporated herein by reference.

Examples of suitable corticosteroids include one or more of alclometasone, amcinonide, amelometasone, beclomethasone, betamethasone, budesonide, ciclesonide, clobetasol, cloticasone, cyclomethasone, deflazacort, deprodone, dexbudesonide, diflorasone, difluprednate, fluticasone, flunisolide, halometasone, halopredone, hydrocortisone, hydrocortisone, methylprednisolone, mometasone, prednicarbate, prednisolone, rimexolone, tixocortol, triamcinolone, ulobetasol, and pharmaceutically acceptable salts, solvates thereof, or mixtures thereof.

Suitable PDE-TV inhibitors may be any PDE-IV inhibitors described in the art or subsequently discovered. For example, PDE-IV inhibitors may include, but are not limited to, one or more compounds disclosed in WO 20051021515, co-pending Indian Patent Application No. 303/DEL/2005; enprofylline, roflumilast, ariflo, Bay-198004, CP-325366 (WO 96/39408), BY343 (WO 98/21208), D-4396 (Sch-351591) (WO 00/26208), V-11294A, Z-15370 (WO 00/05218), and AWD-12-281 (WO 99/55696).

Other examples of PDE-IV inhibitors include compounds selected from:
3-[3-{[(3S)-1-Benzylpyrrolidin-3-yl]oxy}-4-(difluoromethoxy)phenyl]-1,7-dioxa-2-azaspiro[4.4]non-2-ene (Compound No. 1a),
3-[2-(Difluoromethoxy)-5-(1,7-dioxa-2-azaspiro[4.4]non-2-en-3-yl)phenoxy]propan-1-ol (Compound No. 2a),
[2-(Difluoromethoxy)-5-(1,7-dioxa-2-azaspiro[4.4]non-2-en-3-yl)phenoxy]acetonitrile (Compound No. 3a),
4-[(5S or 5R)-1,7-dioxa-2-azaspiro[4.4]non-2-en-3-yl]-2-methoxyphenol (Compound No. 4a),
4-[(5R or 5S)-1,7-Dioxa-2-azaspiro[4.4]non-2-en-3-yl]-2-methoxyphenol (Compound No. 5a),
5-[(5S or 5R)-1,7-Dioxa-2-azaspiro[4.4]non-2-en-3-yl]-2-methoxyphenol (Compound No. 6a),
(5S or 5R)-3-(3,4-Dimethoxyphenyl)-1,7-dioxa-2-azaspiro[4.4]non-2-ene (Compound No. 7a),
(5R or 5S)-3-(3,4-Dimethoxyphenyl)-1,7-dioxa-2-azaspiro[4.4]non-2-ene (Compound No. 8a),
2-(Benzyloxy)-4-(1,7-dioxa-2-azaspiro[4.4]non-2-en-3-yl)phenol (Compound No. 9a),
2-[2-(Difluoromethoxy)-5-(1,7-dioxa-2-azaspiro[4.4]non-2-en-3-yl)phenoxy]ethanol (Compound No. 10a),
3-[4-(Difluoromethoxy)-3-ethoxyphenyl]-1,7-dioxa-2-azaspiro[4.4]non-2-ene (Compound No. 11a),
3-[3-(Cyclohexyloxy)-4-(difluoromethoxy)phenyl]-1,7-dioxa-2-azaspiro[4.4]non-2-ene (Compound No. 12a),
(5R or 5S)-3-[4-(Difluoromethoxy)-3-methoxyphenyl]-1,7-dioxa-2-azaspiro[4.4]non-2-ene (Compound No. 13a),
(5S or 5R)-3-[4-(Difluoromethoxy)-3-methoxyphenyl]-1,7-dioxa-2-azaspiro[4.4]non-2-ene (Compound No. 14a),
Ethyl [2-(difluoromethoxy)-5-(1,7-dioxa-2-azaspiro[4.4]non-2-en-3-yl)phenoxy]acetate (Compound No. 15a),
3-[4-(Difluoromethoxy)-3-(2-morpholin-4-ylethoxy)phenyl]-1,7-dioxa-2-azaspiro[4.4]non-2-ene (Compound No. 16a),
2-(Difluoromethoxy)-5-(1,7-dioxa-2-azaspiro[4.4]non-2-en-3-yl)phenyl cyclohexanecarboxylate (Compound No. 17a),
5-[2-(Difluoromethoxy)-5-(1,7-dioxa-2-azaspiro[4.4]non-2-en-3-yl)phenoxy]pentanoic acid (Compound No. 18a),
3-[3-(2,2,2-Trifluoroethoxy)-4-(difluoromethoxy)phenyl]-1,7-dioxa-2-azaspiro[4.4]non-2-ene (Compound No. 19a),
3-[3-(Cyclopentylmethoxy)-4-(difluoromethoxy)phenyl]-1,7-dioxa-2-azaspiro[4.4]non-2-ene (Compound No. 20a),
N-cyclopropyl-2-[2-(difluoromethoxy)-5-(1,7-dioxa-2-azaspiro[4.4]non-2-en-3-yl)phenoxy]acetamide (Compound No. 21a),
2-[2-(Difluoromethoxy)-5-(1,7-dioxa-2-azaspiro[4.4]non-2-en-3-yl)phenoxy]acetamide (Compound No. 22a),
2-[2-(Difluoromethoxy)-5-(1,7-dioxa-2-azaspiro[4.4]non-2-en-3-yl)phenoxy]-N-methylacetamide (Compound No. 23a),
3-[3-(Cyclopentyloxy)-4-(2,2,2-trifluoroethoxy)phenyl]-1,7-dioxa-2-azaspiro[4.4]non-2-ene (Compound No. 24a),
2-(Difluoromethoxy)-5-(1,7-dioxa-2-azaspiro[4.4]non-2-en-3-yl)phenyl cyclopropanecarboxylate (Compound No. 25a),
2-(Difluoromethoxy)-5-(1,7-dioxa-2-azaspiro[4.4]non-2-en-3-yl)phenyl morpholine-4-carboxylate (Compound No. 26a),
2-(Difluoromethoxy)-5-(1,7-dioxa-2-azaspiro[4.4]non-2-en-3-yl)phenyl benzoate (Compound No. 27a),
5-[2-(Difluoromethoxy)-5-(1,7-dioxa-2-azaspiro[4.4]non-2-en-3-yl)phenoxy] pentanamide (Compound No. 28a),
3-[3-Propoxy-4-(2,2,2-trifluoroethoxy)phenyl]-1,7-dioxa-2-azaspiro[4.4]non-2-ene (Compound No. 29a),
3-[3-Isopropoxy-4-(2,2,2-trifluoroethoxy)phenyl]-1,7-dioxa-2-azaspiro[4.4]non-2-ene (Compound No. 30a),
3-[3-(Cyclopropylmethoxy)-4-(2,2,2-trifluoroethoxy)phenyl]-1,7-dioxa-2-azaspiro[4.4]non-2-ene (Compound No. 31a),
3-[3-(2,3-Dihydro-1H-inden-2-yloxy)-4-(2,2,2-trifluoroethoxy)phenyl]-1,7-dioxa-2-azaspiro[4.4]non-2-ene (Compound No. 32a),
5-(1,7-Dioxa-2-azaspiro[4,4]non-2-en-3-yl)-2-(2,2,2-trifluoroethoxy)phenol (Compound No. 33a),
3-[3-Methoxy-4-(2,2,2-trifluoroethoxy)phenyl]-1,7-dioxa-2-azaspiro[4.4]non-2-ene (Compound No. 34a),
3-[3-Ethoxy-4-(2,2,2-trifluoroethoxy)phenyl]-1,7-dioxa-2-azaspiro[4.4]non-2-ene (Compound No. 35a),
3-[3-Butoxy-4-(2,2,2-trifluoroethoxy)phenyl]-1,7-dioxa-2-azaspiro[4.4]non-2-ene10019955 (Compound No. 36a),
3-[3-(Cyclohexylmethoxy)-4-(2,2,2-trifluoroethoxy)phenyl]-1,7-dioxa-2-azaspiro[4.4]non-2-ene (Compound No. 37a),
3-{[2-(Difluoromethoxy)-5-(1,7-dioxa-2-azaspiro[4.4]non-2-en-3-yl)phenoxy]methyl} benzonitrile (Compound No. 38a),
2-{2-[2-(difluoromethoxy)-5-(1,7-dioxa-2-azaspiro[4.4]non-2-en-3-yl)phenoxy]ethyl}-1H-isoindole-1,3(2H)-dione (Compound No. 39a),
3-[3-(Cyclohexyloxy)-4-(2,2-trifluoroethoxy)phenyl]-1,7-dioxa-2-azaspiro[4.4]non-2-ene (Compound No. 40a),
Ethyl [5-(1,7-dioxa-2-azaspiro[4.4]non-2-en-3-yl)-2-(2,2,2-trifluoroethoxy)phenoxy]acetate (Compound No. 41a),
3-[3-(Cyclohexylmethoxy)-4-(difluoromethoxy)phenyl]-1,7-dioxa-2-azaspiro[4.4]non-2-ene (Compound No. 42a),
Tert-butyl [2-(difluoromethoxy)-5-(1,7-dioxa-2-azaspiro[4.4]non-2-en-3-yl)phenoxy]acetate (Compound No. 43a),
N-cyclopropyl-2-[5-(1,7-dioxa-2-azaspiro[4.4]non-2-en-3-yl)-2-(2,2,2-trifluoroethoxy)phenoxy]acetamide (Compound No. 44a),
2-(Cyclopentyloxy)-4-[(5R or 5S)-1,7-dioxa-2-azaspiro[4.4]non-2-en-3-yl]phenol (Compound No. 45a),
2-(Cyclopentyloxy)-4-[(5S or 5R)-1,7-dioxa-2-azaspiro[4.4]non-2-en-3-yl]phenol (Compound No. 46a),
N-benzyl-2-[5-(1,7-dioxa-2-azaspiro[4.4]non-2-en-3-yl)-2-(2,2,2-trifluoroethoxy)phenoxy]acetamide (Compound No. 47a),
N-Cyclopentyl-2-[5-(1,7-dioxa-2-azaspiro[4.4]non-2-en-3-yl)-2-(2,2,2-trifluoroethoxy)phenoxy]acetamide (Compound No. 48a),
Tert-butyl 4-[2-(difluoromethoxy)-5-(1,7-dioxa-2-azaspiro[4.4]non-2-en-3-yl)phenoxy]piperidine-1-carboxylate (Compound No. 49a),
Hydrochloride salt of 3-[4-(difluoromethoxy)-3-(piperidin-4-yloxy)phenyl]-1,7-dioxa-2-azaspiro[4.4]non-2-ene (Compound No. 50a),
3-{3-[(1-Acetylpiperidin-4-yl)oxy]-4-(difluoromethoxy)phenyl}-1,7-dioxa-2-azaspiro [4.4]non-2-ene (Compound No. 51a),
Tert-butyl (3S)-3-[2-(difluoromethoxy)-5-(1,7-dioxa-2-azaspiro[4.4]non-2-en-3-yl)phenoxy]pyrrolidine-1-carboxylate (Compound No. 52a),
Tert-butyl (3R)-3-[2-(difluoromethoxy)-5-(1,7-dioxa-2-azaspiro[4.4]non-2-en-3-yl)phenoxy]pyrrolidine-1-carboxylate (Compound No. 53a),
Tert-butyl 3-[2-(difluoromethoxy)-5-(1,7-dioxa-2-azaspiro[4.4]non-2-en-3-yl)phenoxy]piperidine-1-carboxylate (Compound No. 54a),
Tert-butyl (2S)-2-{[2-(difluoromethoxy)-5-(1,7-dioxa-2-azaspiro[4.4]non-2-en-3-yl)phenoxy]methyl}pyrrolidine-1-carboxylate (Compound No. 55a),
(5R or 5S)-3-[3-(cyclopentyloxy)-4-(difluoromethoxy)phenyl]-1,7-dioxa-2-azaspiro[4.4]non-2-ene (Compound No. 56a),
(5S or 5R)-3-(3-isopropoxy-4-methoxyphenyl)-1,7-dioxa-2-azaspiro[4.4]non-2-ene (Compound No. 57a),
(5S or 5R)-3-[3-(Cyclopropylmethoxy)-4-methoxyphenyl]-1,7-dioxa-2-azaspiro[4.4]non-2-ene (Compound No. 58a),
2-(Cyclopropylmethoxy)-4-[(5S or 5R)-1,7-dioxa-2-azaspiro[4.4]non-2-en-3-yl]phenol (Compound No. 59a),
4-[(5S or 5R)-1,7-Dioxa-2-azaspiro[4.4]non-2-en-3-yl]-2-isopropoxyphenol (Compound No. 60a),
(5S or 5R)-3-[3-(cyclopentyloxy)-4-(difluoromethoxy)phenyl]-1,7-dioxa-2-azaspiro[4.4]non-2-ene (Compound No. 61a),
(5S or 5R)-3-[3-(Cyclopropylmethoxy)-4-(difluoromethoxy)phenyl]-1,7-dioxa-2-azaspiro[4.4]non-2-ene (Compound No. 62a),
(5S or 5R)-3-[4-(difluoromethoxy)-3-isopropoxyphenyl]-1,7-dioxa-2-azaspiro[4.4]non-2-ene (Compound No. 63a),
(5R or 5S)-3-[4-(difluoromethoxy)-3-isopropoxyphenyl]-1,7-dioxa-2-azaspiro[4.4]non-2-ene (Compound No. 64a),
2-(Cyclopropylmethoxy)-4-[(5R or 5S)-1,7-dioxa-2-azaspiro[4.4]non-2-en-3-yl]phenol (Compound No. 65a),
4-[(5R or 5S)-1,7-Dioxa-2-azaspiro[4.4]non-2-en-3-yl]-2-isopropoxyphenol (Compound No. 66a),
(5R or 5S)-3-[3-(Cyclopropylmethoxy)-4-(difluoromethoxy)phenyl]-1,7-dioxa-2-azaspiro[4.4]non-2-ene (Compound No. 67a),
(5R or S8)-3-[4-(difluoromethoxy)-3-isopropoxyphenyl]-1,7-dioxa-2-azaspiro[4.4]non-2-ene (Compound No. 68a),
Hydrochloride salt of 3-{4-(difluoromethoxy)-3-[(3S)-pyrrolidin-3-yloxy]phenyl}-1,7-dioxa-2-azaspiro[4.4]non-2-ene (Compound No. 69a),
Hydrochloride salt of 3-{4-(difluoromethoxy)-3-[(2S)-pyrrolidin-2-ylmethoxy]phenyl}-1,7-dioxa-2-azaspiro[4.4]non-2-ene (Compound No. 70a),
Hydrochloride salt of 3-{4-(difluoromethoxy)-3-[(2R)-pyrrolidin-2-ylmethoxy]phenyl}-1,7-dioxa-2-azaspiro[4.4]non-2-ene (Compound No. 71a),
3-[4-(Difluoromethoxy)-3-{[(2R)-1-propionylpyrrolidin-2-yl]methoxy}phenyl]-1,7-dioxa-2-azaspiro[4.4]non-2-ene (Compound No. 72a),
3-[3-{[(2S)-1-acetylpyrrolidin-2-yl]methoxy}-4-(difluoromethoxy)phenyl]-1,7-dioxa-2-azaspiro[4.4]non-2-ene (Compound No. 73a),
3-[3-{[(3S)-1-benzoylpyrrolidin-3-yl]oxy}-4-(difluoromethoxy)phenyl]-1,7-dioxa-2-azaspiro[4.4]non-2-ene (Compound No. 74a),
3-[4-(Difluoromethoxy)-3-{[(3S)-1-propionylpyrrolidin-3-yl]oxy}phenyl]-1,7-dioxa-2-azaspiro[4.4]non-2-ene (Compound No. 75a),
(5S or 5R)-3-[3-(Benzyloxy)-4-(difluoromethoxy)phenyl]-1,7-dioxa-2-azaspiro[4.4]non-2-ene (Compound No. 76a),
2-(Benzyloxy)-4-[(5S or 5R)-1,7-dioxa-2-azaspiro[4.4]non-2-en-3-yl]phenol (Compound No. 77a),
(5S or 5R)-3-[3-(Benzyloxy)-4-methoxyphenyl]-1,7-dioxa-2-azaspiro[4.4]non-2-ene (Compound No. 78a),
3-{4-(Difluoromethoxy)-3-[(1-propionylpiperidin-4-yl)oxy]phenyl}-1,7-dioxa-2-azaspiro[4.4]non-2-ene (Compound No. 79a),
3-{4-(Difluoromethoxy)-3-([1-(4-fluorobenzoyl)piperidin-4-yl]oxy}phenyl]-1,7-dioxa-2-azaspiro[4.4]non-2-ene (Compound No. 80a),
3-[3-{[1-(Cyclopropylcarbonyl)piperidin-4-yl]oxy}-4-(difluoromethoxy)phenyl]-1,7-dioxa-2-azaspiro[4.4]non-2-ene (Compound No. 81a),
3-[3-{[1-(Cyclopentylcarbonyl)piperidin-4-yl]oxy}-4-(difluoromethoxy)phenyl]-1,7-dioxa-2-azaspiro[4.4]non-2-ene (Compound No. 82a),
3-[4-(Difluoromethoxy)-3-({1-[(trifluoromethyl)sulfonyl]piperidin-4-yl}oxy)phenyl]-1,7-dioxa-2-azaspiro[4.4]non-2-ene (Compound No. 83a),
3-{3-[(1-Acetylpiperidin-3-yl)oxy]-4-(difluoromethoxy)phenyl}-1,7-dioxa-2-azaspiro[4.4]non-2-ene (Compound No. 84a),
3-{4-(Difluoromethoxy)-3-[(1-propionylpiperidin-3-yl)oxy]phenyl}-1,7-dioxa-2-azaspiro[4.4]non-2-ene (Compound No. 85a),
3-[4-(Difluoromethoxy)-3-{[1-(4-fluorobenzoyl)piperidin-3-yl]oxy}phenyl]-1,7-dioxa-2-azaspiro[4.4]non-2-ene (Compound No. 86a),
3-[3-{[1-(Cyclopropylcarbonyl)piperidin-3-yl]oxy}-4-(difluoromethoxy)phenyl]-1,7-dioxa-2-azaspiro[4.4]non-2-ene (Compound No. 87a),
3-[3-{[1-(Cyclopentylcarbonyl)piperidin-3-yl]oxy}-4-(difluoromethoxy)phenyl]-1,7-dioxa-2-azaspiro[4.4]non-2-ene (Compound No. 88a),
3-[4-(Difluoromethoxy)-3-{[1-(ethylsulfonyl)piperidin-3-yl]oxy}phenyl]-1,7-dioxa-2-azaspiro[4.4]non-2-ene (Compound No. 89a),
3-[3-(Benzyloxy)-4-(2,2,2-trifluoroethoxy)phenyl]-1,7-dioxa-2-azaspiro[4.4]non-2-ene (Compound No. 90a),
2-(Difluoromethoxy)-5-[(5S or 5R)-1,7-dioxa-2-azaspiro[4.4]non-2-en-3-yl]phenol (Compound No. 91a),
5-[(5R or 5S)-1,7-Dioxa-2-azaspiro[4.4]non-2-en-3-yl]-2-methoxyphenol (Compound No. 92a)
   and any pharmaceutically acceptable acid addition salts thereof.

Other suitable PDE-IV inhibitors (disclosed in co-pending Indian Patent Application No. 303/DEL2005) include, for example:
3-[3-(cyclopentyloxy)-4-methoxyphenyl]-1,7-dioxa-2-azaspiro[4.4]non-2-en-6-ol (Compound No. 1aa),
3-[3-(Cyclopentyloxy)-4-methoxyphenyl]-N-(4-fluorophenyl)-1-oxa-2,7-diazaspiro[4.4]non-2-ene-7-carboxamide (Compound No. 2aa),
3-[3-(Cyclopentyloxy)-4-methoxyphenyl]-7-(tetrahydrofuran-3-ylcarbonyl)-1-oxa-2,7-diazaspiro[4.4]non-2-ene (Compound No. 3aa),
3-[3-(cyclopentyloxy)-4-methoxyphenyl]-N,N-dimethyl-1-oxa-2,7-diazaspiro[4.4]non-2-ene-7-sulfonamide (Compound No. 4aa),
N-butyl-3-[3-(cyclopentyloxy)-4-methoxyphenyl]-1-oxa-2,7-diazaspiro[4.4]non-2-ene-7-carboxamide (Compound No. 5aa),
2-{3-[3-(Cyclopentyloxy)-4-methoxyphenyl]-1-oxa-2,7-diazaspiro[4.4]non-2-en-7-yl}acetamide (Compound No. 6aa),
Hydrochloride salt of 3-[3-(cyclopentyloxy)-4-methoxyphenyl]-8-prolyl-1-oxa-2,8-diazaspiro[4.5]dec-2-ene (Compound No. 7aa),
3-[3-(Cyclopentyloxy)-4-methoxyphenyl]-8-(2-morpholin-4-yl-ethyl)-1-oxa-2,8-diazaspiro[4.5]dec-2-ene (Compound No. 8aa),
N-butyl-3-[3-(cyclopentyloxy)-4-methoxyphenyl]-1-oxa-2,8-diazaspiro[4.5]dec-2-ene-8-carboxamide (Compound No. 9aa),
3-[3-(cyclopentyloxy)-4-methoxyphenyl]-8-(methylsulfonyl)-1-oxa-2,8-diazaspiro[4.5]dec-2-ene (Compound No. 10aa),
3-[3-(cyclopentyloxy)-4-methoxyphenyl]-1-oxa-2-azaspiro[4.4]non-2-ene (Compound No. 11aa),
3-[3,4-bis(2-morpholin-4-ylethoxy)phenyl]-1,7-dioxa-2-azaspiro[4.4]non-2-ene (Compound No. 12aa),
3-(3,4-diisopropoxyphenyl)-1,7-dioxa-2-azaspiro[4.4]non-2-ene (Compound No. 13aa),
3-[3-methoxy-4-(2-morpholin-4-ylethoxy)phenyl]-1,7-dioxa-2-azaspiro[4.4]non-2-ene (Compound No. 14aa),
3-[3-(cyclopentyloxy)-4-methoxyphenyl]-1,7-dioxa-2-azaspiro[4.4]non-2-en-8-one (Compound No. 15aa),
3-[3-(cyclopentyloxy)-4-methoxyphenyl]-1,7-dioxa-2-azaspiro[4.4]non-2-en-8-ol (Compound No. 16aa),
3-[3-(Cyclopentyloxy)-4-methoxyphenyl]-7-isopropyl-1-oxa-2,7-diazaspiro[4.4] non-2-ene (Compound No. 17aa),
3-[3-(cyclopentyloxy)-4-methoxyphenyl]-7-(cyclopropylcarbonyl)-1-oxa-2,7-diazaspiro[4.4]non-2-ene (Compound No. 18aa),
N-benzyl-3-[3-(cyclopentyloxy)-4-methoxyphenyl]-1-oxa-2,7-diazaspiro[4.4]non-2-ene-7-carboxamide (Compound No. 19aa),
7-acetyl-3-[3-(cyclopentyloxy)-4-methoxyphenyl]-1-oxa-2,7-diazaspiro[4.4]non-2-ene (Compound No. 20aa),
Tert-butyl 3-[3-(cyclopentyloxy)-4-methoxyphenyl]-1-oxa-2,7-diazaspiro[4.5]dec-2-ene-7-carboxylate (Compound No. 21aa),
N-butyl-N-{3-[3-(cyclopentyloxy)-4-methoxyphenyl]-1-oxa-2-azaspiro[4.5]dec-2-en-8-yl}urea (Compound No. 22aa),
N-{3-[3-(cyclopentyloxy)-4-methoxyphenyl]-1-oxa-2-azaspiro[4.5]dec-2-en-8-yl}-N′-(2-methoxyphenyl)urea (Compound No. 23aa),
3-[3-(cyclopentyloxy)-4-methoxyphenyl]-1-oxa-2-azaspiro[4.5]dec-2-en-8-ol (Compound No. 24
Hydrochloride salt of 3-[3-(cyclopentyloxy)-4-methoxyphenyl]-1-oxa-2,7-diazaspiro[4.5]dec-2-ene (Compound No. 25aa),
3-[3-(cyclopentyloxy)-4-methoxyphenyl]-1-oxa-2-azaspiro[4.5]dec-2-en-8-one (Compound No. 26aa),
3-[3,4-bis(cyclopentyloxy)phenyl]-1,7-dioxa-2-azaspiro[4.4]non-2-ene (Compound No. 27aa),
3-[3,4-Bis(cyclopropylmethoxy)phenyl]-1,7-dioxa-2-azaspiro[4.4]non-2-ene (Compound No. 28aa),
3-[3-(cyclopentyloxy)-4-methoxyphenyl]-1,7-dioxa-2-azaspiro[4.4]non-2-en-4-ol (Compound No. 29aa),
(R)-3-[3-(cyclopentyloxy)-4-methoxyphenyl]-1,7-dioxa-2-azaspiro[4.4]non-2-ene (Compound No. 30aa),
3-[3-(Cyclopentyloxy)-4-methoxyphenyl]-8-(cyclopropylmethyl)-1-oxa-2,8-diazaspiro[4.5]dec-2-ene (Compound No. 31aa),
N-Benzyl-3-[3-(cyclopentyloxy)-4-methoxyphenyl]-1-oxa-2,8-diazaspiro[4.5]dec-2-ene-8-carboxamide (Compound No. 32aa),
3-[3,4-Bis(benzyloxy)phenyl]-1,7-dioxa-2-azaspiro[4.4]non-2-ene (Compound No. 33aa),
4-(1,7-Dioxa-2-azaspiro[4.4]non-2-en-3-yl)benzene-1,2-diol (Compound No. 34aa),
7-Amino-3-[3-(cyclopentyloxy)-4-methoxyphenyl]-1-oxa-2,7-diazaspiro[4.4]non-2-en-6-one (Compound No. 35aa),
Ethyl 8-benzyl-3-[3-(cyclopentyloxy)-4-methoxyphenyl]-1-oxa-2,8-diazaspiro[4.5]dec-2-ene-4-carboxylate (Compound No. 36aa),
3-[3-(Cyclopentyloxy)-4-methoxyphenyl]-1-oxa-2-azaspiro[4.5]dec-2-ene-4-carboxylic acid (Compound no. 37aa),
8-Benzyl-3-[3-(cyclopentyloxy)-4-methoxyphenyl]-1-oxa-2,8-diazaspiro[4.5]dec-2-ene (Compound No. 38aa),
Ethyl 3-[3-(cyclopentyloxy)-4-methoxyphenyl]-1-oxa-2-azaspiro[4.5]dec-2-ene-4-carboxylate (Compound No. 39aa),
3-[3-(Difluoromethoxy)-4-methoxyphenyl]-1,7-dioxa-2-azaspiro[4.4]non-2-ene (Compound No. 40aa),
2-(Difluoromethoxy)-5-(1,7-dioxa-2-azaspiro[4.4]non-2-en-3-yl)phenol (Compound No. 41aa),
3-[3-(Cyclopentyloxy)-4-methoxyphenyl]-1-oxa-2,7-diazaspiro[4.4]non-2-en-6-one (Compound No. 42aa),
3-[3-(Cyclopentyloxy)-4-methoxyphenyl]-3a,6a-dimethyl-3aH-cyclopenta[d]isoxazole-4,6(5H,6aH)-dione (Compound No. 43aa),
3-[3-(Cyclopentyloxy)-4-methoxyphenyl]-3a,4,6,6a-tetrahydrofuro[3,4-d]isoxazole (Compound No. 44aa),
3-[3-(Cyclopentyloxy)-4-methoxyphenyl]-6,6a-dihydrofuro[3,4-d]isoxazol-4(3aH)-one (Compound No. 45aa),
Tert-butyl [({3-[3-(cyclopentyloxy)-4-methoxyphenyl]-1-oxa-2-azaspiro[4.5]dec-2-en-8-yl}amino)carbonyl]carbamate (Compound No. 46aa),
N-{3-[3-(Cyclopentyloxy)-4-methoxyphenyl]-1-oxa-2-azaspiro[4.5]dec-2-en-8-yl}cyclopentanecarboxamide (Compound No. 47aa),
8-Acetyl-3-[3-(cyclopentyloxy)-4-methoxyphenyl]-1-oxa-2,8-diazaspiro[4.5]dec-2-ene (Compound No. 48aa),
8-(Cyclopentylcarbonyl)-3-[3-(cyclopentyloxy)-4-methoxyphenyl]-1-oxa-2,8-diazaspiro[4.5]dec-2-ene (Compound No. 49aa),
3-[3-(Cyclopentyloxy)-4-methoxyphenyl]-8-(2-piperidin-1-ylethyl)-1-oxa-2,8-diazaspiro[4.5]dec-2-ene (Compound No. 50aa),
3-(2,3-Dihydro-1,4-benzodioxin-6-yl)-1,7-dioxa-2-azaspiro[4.4]non-2-ene (Compound No. 51aa),
3-[3-(Cyclopentyloxy)-4-methoxyphenyl]-1,8-dioxa-2-azaspiro[4.5]dec-2-ene (Compound No. 52aa),
3-[3-(Cyclopentyloxy)-4-methoxyphenyl]-3aH-cyclopenta[d]isoxazole-4,6(5H,6aH)-dione (Compound No. 53aa),
3-[3-(Cyclopentyloxy)-4-methoxyphenyl]-8-ethyl-1-oxa-2,8-diazaspiro[4.5]dec-2-ene (Compound No. 54aa),
3-[3-(Cyclopentyloxy)-4-methoxyphenyl]-8-vinyl-1-oxa-2-azaspiro[4.5]dec-2-en-8-ol (Compound No. 55aa),
3-[3-(Cyclopentyloxy)-4-methoxyphenyl]-3a,4,5,6,7,7a-hexahydro-1,2-benzisoxazole (Compound No. 56aa),
3-[3-(Cyclopentyloxy)-4-methoxyphenyl]-4,5,6,6a-tetrahydro-3aH-cyclopenta[d]isoxazole (Compound No. 57aa),
N-{3-[3-(Cyclopentyloxy)-4-methoxyphenyl]-1-oxa-2-azaspiro[4.5]dec-2-en-8-yl}methanesulfonamide (Compound No. 58aa),
3-[3-(Cyclopentyloxy)-4-methoxyphenyl]-8-methyl-1-oxa-2-azaspiro[4.5]dec-2-en-8-ol (Compound No. 59aa),
3-[3-(Allyloxy)-4-methoxyphenyl]-1,7-dioxa-2-azaspiro[4.4]non-2-ene (Compound No. 60aa),
3-[3-(2-Chloroethoxy)-4-methoxyphenyl]-1,7-dioxa-2-azaspiro[4.4]non-2-ene (Compound No. 61aa),
2-(Cyclopentyloxy)-4-(1,7-dioxa-2-azaspiro[4.4]non-2-en-3-yl)phenol (Compound No. 62aa),
3-(4-Butoxy-3-isobutoxyphenyl)-1,7-dioxa-2-azaspiro[4.4]non-2-ene (Compound No. 63aa),
3-(3-Isobutoxy-4-propoxyphenyl)-1,7-dioxa-2-azaspiro[4.4]non-2-ene (Compound No. 64aa),
3-[3-Butoxy-4-(cyclopropylmethoxy)phenyl]-1,7-dioxa-2-azaspiro[4.4]non-2-ene (Compound No. 65aa),
3-(3-Butoxy-4-ethoxyphenyl)-1,7-dioxa-2-azaspiro[4.4]non-2-ene (Compound No. 66aa),
3-[3-Butoxy-4-(cyclohexyloxy)phenyl]-1,7-dioxa-2-azaspiro[4.4]non-2-ene (Compound No. 67aa),
3-[3-(Cyclohexylmethoxy)-4-ethoxyphenyl]-1,7-dioxa-2-azaspiro[4.4]non-2-ene (Compound No. 68aa),
3-[3-(Cyclohexylmethoxy)-4-isopropoxyphenyl]-1,7-dioxa-2-azaspiro[4.4]non-2-ene (Compound No. 69aa),
3-[4-Butoxy-3-(cyclohexylmethoxy)phenyl]-1,7-dioxa-2-azaspiro[4.4]non-2-ene (Compound No. 70aa),
3-(4-Isobutoxy-3-isopropoxyphenyl)-1,7-dioxa-2-azaspiro[4.4]non-2-ene (Compound No. 71aa),
3-(4-Butoxy-3-isopropoxyphenyl)-1,7-dioxa-2-azaspiro[4.4]non-2-ene (Compound No. 72aa),
3-[4-(Cyclohexylmethoxy)-3-isopropoxyphenyl]-1,7-dioxa-2-azaspiro[4.4]non-2-ene (Compound No. 73aa),
3-[3-Isopropoxy-4-(2-morpholin-4-ylethoxy)phenyl]-1,7-dioxa-2-azaspiro[4.4]non-2-ene (Compound No. 74aa),
3-[3-(Cyclopropylmethoxy)-4-isopropoxyphenyl]-1,7-dioxa-2-azaspiro[4.4]non-2-ene (Compound No. 75aa),
3-[3-(Cyclopropylmethoxy)-4-(2-morpholin-4-ylethoxy)phenyl]-1,7-dioxa-2-azaspiro[4.4]non-2-ene (Compound No. 76aa),
3-[4-Butoxy-3-(cyclopropylmethoxy)phenyl]-1,7-dioxa-2-azaspiro[4.4]non-2-ene (Compound No. 77aa),
3-[3-(Cyclopropylmethoxy)-4-isopropoxyphenyl]-1,7-dioxa-2-azaspiro[4.4]non-2-ene (Compound No. 78aa),
3-(3-Isobutoxy-4-isopropoxyphenyl)-1,7-dioxa-2-azaspiro[4.4]non-2-ene (Compound No. 79aa),
3-[4-(Cyclopropylmethoxy)-3-isobutoxyphenyl]-1,7-dioxa-2-azaspiro[4.4]non-2-ene (Compound No. 80aa),
3-[4-(cyclohexyloxy)-3-(cyclopentyloxy)phenyl]-1,7-dioxa-2-azaspiro[4.4]non-2-ene (Compound No. 81aa),
3-[4-(Cyclohexylmethoxy)-3-(cyclopentyloxy)phenyl]-1,7-dioxa-2-azaspiro[4.4]non-2-ene (Compound No. 82aa),
3-[4-(Cyclopropylmethoxy)-3-(cyclopentyloxy)phenyl]-1,7-dioxa-2-azaspiro[4.4]non-2-ene (Compound No. 83aa),
3-[3-(Cyclopentyloxy)-4-isobutoxyphenyl]-1,7-dioxa-2-azaspiro[4.4]non-2-ene (Compound No. 84aa),
3-[3-(Cyclopentyloxy)-4-ethoxyphenyl]-1,7-dioxa-2-azaspiro[4.4]non-2-ene (Compound No. 8S5aa),
3-[3-(Cyclopropylmethoxy)-4-ethoxyphenyl]-1,7-dioxa-2-azaspiro[4.4]non-2-ene (Compound No. 86aa),
3-[4-(Cyclopentyloxy)-3-isobutoxyphenyl]-1,7-dioxa-2-azaspiro[4.4]non-2-ene (Compound No. 87aa),
3-[3-Isopropoxy-4-(2-morpholin-4-ylethoxy)phenyl]-1,7-dioxa-2-azaspiro[4.4]non-2-ene (Compound No. 88aa),
3-(4-Ethoxy-3-isobutoxyphenyl)-1,7-dioxa-2-azaspiro[4.4]non-2-ene (Compound No. 89aa),
3-[3-(Cyclopentyloxy)-4-propoxyphenyl]-1,7-dioxa-2-azaspiro[4.4]non-2-ene (Compound No. 90aa),
3-[4-Butoxy-3-(cyclopentyloxy)phenyl]-1,7-dioxa-2-azaspiro[4.4]non-2-ene (Compound No. 91aa),
3-[3-(Cyclopentyloxy)-4-isopropoxyphenyl]-1,7-dioxa-2-azaspiro[4.4]non-2-ene (Compound No. 92aa),
3-[3-(Cyclopentyloxy)-4-(cycloheptyloxy)phenyl]-1,7-dioxa-2-azaspiro[4.4]non-2-ene (Compound No. 93aa),
3-[3-(Cyclopentyloxy)-4-(2-morpholin-4-ylethoxy)phenyl]-1,7-dioxa-2-azaspiro[4.4]non-2-ene (Compound No. 94aa),
3-[4-(Cyclohexylmethoxy)-3-isobutoxyphenyl]-1,7-dioxa-2-azaspiro[4.4]non-2-ene (Compound No. 95aa),
3-[4-(Cyclohexylmethoxy)-3-(cyclopropylmethoxy)phenyl]-1,7-dioxa-2-azaspiro[4.4]non-2-ene (Compound No. 96aa),
3-[3-(Cyclopropylmethoxy)-4-propoxyphenyl]-1,7-dioxa-2-azaspiro[4.4]non-2-ene (Compound No. 97aa),
3-[4-(Cyclopentyloxy)-3-(cyclopropylmethoxy)phenyl]-1,7-dioxa-2-azaspiro[4.4]non-2-ene (Compound No. 98aa),
3-[4-(Cyclopropylmethoxy)-3-isopropoxyphenyl]-1,7-dioxa-2-azaspiro[4.4]non-2-ene (Compound No. 99aa),
3-[4-(Cyclopentyloxy)-3-isopropoxyphenyl]-1,7-dioxa-2-azaspiro[4.4]non-2-ene (Compound No. 100aa),
3-(3-Isopropoxy-4-propoxyphenyl)-1,7-dioxa-2-azaspiro[4.4]non-2-ene (Compound No. 101aa),
3-(4-Ethoxy-3-isopropoxyphenyl)-1,7-dioxa-2-azaspiro[4.4]non-2-ene (Compound No. 102aa),
3-[3-Butoxy-4-(2-morpholin-4-ylethoxy)phenyl]-1,7-dioxa-2-azaspiro[4.4]non-2-ene (Compound No. 103aa),
3-[3-Butoxy-4-(cyclopentyloxy)phenyl]-1,7-dioxa-2-azaspiro[4.4]non-2-ene (Compound No. 104aa),
3-(3-Butoxy-4-propoxyphenyl)-1,7-dioxa-2-azaspiro[4.4]non-2-ene (Compound No. 105aa),
3-(3-Butoxy-4-isopropoxyphenyl)-1,7-dioxa-2-azaspiro[4.4]non-2-ene (Compound No. 106aa),
3-[3-(Cyclohexylmethoxy)-4-propoxyphenyl]-1,7-dioxa-2-azaspiro[4.4]non-2-ene (Compound No. 107aa),
3-[3-(Cyclohexylmethoxy)-4-isobutoxyphenyl]-1,7-dioxa-2-azaspiro[4.4]non-2-ene (Compound No. 108aa),
3-[3-(Cyclohexylmethoxy)-4-(cyclopentyloxy)phenyl]-1,7-dioxa-2-azaspiro[4.4]non-2-ene (Compound No. 109aa),
3-[3-(Cyclohexylmethoxy)-4-(cyclopropylmethoxy)phenyl]-1,7-dioxa-2-azaspiro[4.4]non-2-ene (Compound No. 110aa),
3-[4-(Cyclohexylmethoxy)-3-propoxyphenyl]-1,7-dioxa-2-azaspiro[4.4]non-2-ene (Compound No. 111aa),
3-[4-(Cyclopropylmethoxy)-3-propoxyphenyl]-1,7-dioxa-2-azaspiro[4.4]non-2-ene (Compound No. 12aa),
3-[4-(Cyclopentyloxy)-3-propoxyphenyl]-1,7-dioxa-2-azaspiro[4.4]non-2-ene (Compound No. 113aa),
3-[4-(3-Isobutoxy)-3-propoxyphenyl]-1,7-dioxa-2-azaspiro[4.4]non-2-ene (Compound No. 114aa),
3-[3-(Cycloheptyloxy)-4-(cyclopropylmethoxy)phenyl]-1,7-dioxa-2-azaspiro[4.4]non-2-ene (Compound No. 115aa),
3-[3-(Cycloheptyloxy)-4-propoxyphenyl]-1,7-dioxa-2-azaspiro[4.4]non-2-ene (Compound No. 116aa),
3-[4-Butoxy-3-(cycloheptyloxy)phenyl]-1,7-dioxa-2-azaspiro[4.4]non-2-ene (Compound No. 117aa),
3-[3-(Cycloheptyloxy)-4-isopropoxyphenyl]-1,7-dioxa-2-azaspiro[4.4]non-2-ene (Compound No. 118aa),
3-[3-(Cycloheptyloxy)-4-(cyclopentyloxy)phenyl]-1,7-dioxa-2-azaspiro[4.4]non-2-ene (Compound No. 119aa),
3-(3-Ethoxy-4-propoxyphenyl)-1,7-dioxa-2-azaspiro[4.4]non-2-ene (Compound No. 120aa),
3-[4-(Cycloheptyloxy)-3-ethoxyphenyl]-1,7-dioxa-2-azaspiro[4.4]non-2-ene (Compound No. 121aa),
3-[4-(Cyclopropylmethoxy)-3-ethoxyphenyl]-1,7-dioxa-2-azaspiro[4.4]non-2-ene (Compound No. 122aa),
3-[4-(Cyclohexylmethoxy)-3-ethoxyphenyl]-1,7-dioxa-2-azaspiro[4.4]non-2-ene (Compound No. 123aa),
(S)-3-[3-(cyclopentyloxy)-4-methoxyphenyl]-1,7-dioxa-2-azaspiro[4.4]non-2-ene (Compound No. 124aa),
3-(3-Butoxy-4-isobutoxyphenyl)-1,7-dioxa-2-azaspiro[4.4]non-2-ene (Compound No. 125aa),
3-(3-Ethoxy-4-isopropoxyphenyl)-1,7-dioxa-2-azaspiro[4.4]non-2-ene (Compound No. 126aa),
3-[4-(Cyclopentyloxy)-3-ethoxyphenyl]-1,7-dioxa-2-azaspiro[4.4]non-2-ene (Compound No. 127aa),
3-(4-Butoxy-3-ethoxyphenyl)-1,7-dioxa-2-azaspiro[4.4]non-2-ene (Compound No. 125aa),
3-(3-Ethoxy-4-isobutoxyphenyl)-1,7-dioxa-2-azaspiro[4.4]non-2-ene (Compound No. 129aa),
3-[3-(Cycloheptyloxy)-4-isobutoxyphenyl]-1,7-dioxa-2-azaspiro[4.4]non-2-ene (Compound No. 130aa),
3-[3-(Cycloheptyloxy)-4-(cyclopentyloxy)phenyl]-1,7-dioxa-2-azaspiro[4.4]non-2-ene (Compound No. 131aa),
3-[3-(Cycloheptyloxy)-4-ethoxyphenyl]-1,7-dioxa-2-azaspiro[4.4]non-2-ene (Compound No. 132aa),
3-(4-Butoxy-3-propoxyphenyl)-1,7-dioxa-2-azaspiro[4.4]non-2-ene (Compound No. 133aa),
3-(4-Ethoxy-3-propoxyphenyl)-1,7-dioxa-2-azaspiro[4.4]non-2-ene (Compound No. 134aa),
3-[4-(Morpholin-4-ylethoxy)-3-propoxyphenyl]-1,7-dioxa-2-azaspiro[4.4]non-2-ene (Compound No. 135aa),
3-(4-Isopropoxy-3-propoxyphenyl)-1,7-dioxa-2-azaspiro[4.4]non-2-ene (Compound No. 136aa),
2-[5-(1,7-Dioxa-2-azaspiro[4.4]non-2-en-3-yl)-2-methoxyphenoxy]cyclopentanol (Compound No. 137aa),
N-{3-[3-(Cyclopentyloxy)-4-m ethoxyphenyl]-1-oxa-2-azaspiro[4.5]dec-2-en-8-yl}-2-fluorobenzamide (Compound No. 138aa),
N-{3-[3-(cyclopentyloxy)-4-methoxyphenyl]-1-oxa-2-azaspiro[4.5]dec-2-en-8-yl}benzamide (Compound No. 139aa),
3-[3-(Cyclopentyloxy)-4-methoxyphenyl]-4,5,6,6a-tetrahydro-3aH-pyrrolo[3,4-d]isoxazole (Compound No. 140aa),
7-(Cyclopentylcarbonyl)-3-[3-(cyclopentyloxy)-4-methoxyphenyl]-1-oxa-2,7-diazaspiro[4.5]dec-2-ene (Compound No. 141aa),
Tert-butyl 3-[3-(cyclopentyloxy)-4-methoxyphenyl]-3a,4,6,6a-tetrahydro-5H-pyrrolo[3,4-d]isoxazole-5-carboxylate (Compound No. 142aa),
3-[3-(Cyclopentyloxy)-4-methoxyphenyl]-1-oxa-2,8-diazaspiro[4.5]dec-2-ene-8-carboxamide (Compound No. 143aa),
N-Butyl-3-[3-(cyclopentyloxy)-4-methoxyphenyl]-1-oxa-2,7-diazaspiro[4.5]dec-2-ene-7-carboxamide (Compound No. 144aa),
3-[3-(Cyclopentyloxy)-4-methoxyphenyl]-7-(methylsulfonyl)-1-oxa-2,7-diazaspiro[4.5]dec-2-ene (Compound No. 145aa),
3-[4-Methoxy-3-(pyridin-3-ylmethoxy)phenyl]-1,7-dioxa-2-azaspiro[4.4]non-2-ene (Compound No. 146aa),
5-Acetyl-3-[3-(cyclopentyloxy)-4-methoxyphenyl]-4,5,6,6a-tetrahydro-3aH-pyrrolo[3,4-d]isoxazole (Compound No. 147aa),
3-[3-(Cyclopentyloxy)-4-methoxyphenyl]-5-(methylsulfonyl)-4,5,6,6a-tetrahydro-3aH-pyrrolo[3,4-d]isoxazole (Compound No. 148aa),
4-Bromo-3-[3-(cyclopentyloxy)-4-methoxyphenyl]-1,7-dioxa-2-azaspiro[4.4]non-2-ene (Compound No. 149aa),
3-[3-(Cyclopentyloxy)-4-methoxyphenyl]-3a,5,6,7a-tetrahydro-1,2-benzisoxazol-7(4H)-one (Compound No. 150aa),
3-[4-(Difluoromethoxy)-3-(2,3-dihydro-1H-inden-2-yloxy)phenyl]-1,7-dioxa-2-azaspiro[4.4]non-2-ene (Compound No. 151aa),
3-[4-(Cyclopentyloxy)-3-(2,3-dihydro-1H-inden-2-yloxy)phenyl]-1,7-dioxa-2-azaspiro[4.4]non-2-ene (Compound No. 152aa),
3-[4-Butoxy-3-(2,3-dihydro-1H-inden-2-yloxy)phenyl]-1,7-dioxa-2-azaspiro[4.4]non-2-ene (Compound No. 153aa),
3-(3-{[3-(Benzyloxy)cyclopentyl]oxy}-4-methoxyphenyl)-1,7-dioxa-2-azaspiro[4.4]non-2-ene (Compound No. 154aa),
7-Acetyl-3-[3-(cyclopentyloxy)-4-methoxyphenyl]-1-oxa-2,7-diazaspiro[4.5]dec-2-ene (Compound No. 155aa),
3-[4-Methoxy-3-(pyridin-2-ylmethoxy)phenyl]-1,7-dioxa-2-azaspiro[4.4]non-2-ene (Compound No. 156aa),
3-[3-(2,3-Dihydro-1H-inden-2-yloxy)-4-ethoxyphenyl]-1,7-dioxa-2-azaspiro[4.4]non-2-ene (Compound No. 157aa),
3-[3-(2,3-Dihydro-1H-inden-2-yloxy)-4-propoxyphenyl]-1,7-dioxa-2-azaspiro[4.4]non-2-ene (Compound No. 158aa),
3-[4-(Cyclopropylmethoxy)-3-(2,3-dihydro-1H-inden-2-yloxy)phenyl]-1,7-dioxa-2-azaspiro[4.4]non-2-ene (Compound No. 159aa),
3-[3-(2,3-Dihydro-1H-inden-2-yloxy)-4-isopropoxyphenyl]-1,7-dioxa-2-azaspiro[4.4]non-2-ene (Compound No. 160aa),
2-(2,3-Dihydro-1H-inden-2-yloxy)-4-(1,7-dioxa-2-azaspiro[4.4]non-2-en-3-yl)phenol (Compound No. 161aa),
N-cyclopropyl-2-[5-(1,7-dioxa-2-azaspiro[4.4]non-2-en-3-yl)-2-methoxyphenoxy]acetamide (Compound No. 162aa),
Hydrochloride salt of 3-[4-methoxy-3-(piperidin-3-yloxy)phenyl]-1,7-dioxa-2-azaspiro[4.4]non-2-ene (Compound No. 163aa),
2-[5-(1,7-Dioxa-2-azaspiro[4.4]non-2-en-3-yl)-2-methoxyphenoxy]acetamide (Compound No. 164aa),
Ethyl [5-(1,7-dioxa-2-azaspiro[4.4]non-2-en-3-yl)-2-methoxyphenoxy]acetate (Compound No. 165aa),
[5-(1,7-Dioxa-2-azaspiro[4.4]non-2-en-3-yl)-2-methoxyphenoxy]acetonitrile (Compound No. 166aa), and
3-{3-[(2,6-Dichloropyridin-4-yl)methoxy]-4-methoxyphenyl}-1,7-dioxa-2-azaspiro[4.4]non-2-ene (Compound No. 167aa),
   and any pharmaceutically acceptable acid addition salts thereof.

Pharmaceutically acceptable acid addition salts include, for example, salts of hydrochloric acid, hydrobromic acid, sulfuric acid, phosphoric acid, methanesulfonic acid, acetic acid, fumaric acid, succinic acid, lactic acid, citric acid, tartaric acid, or maleic acid. In some embodiments, such salts include acetate, hydrochloride, hydrobromide, sulfate, phosphate, and methanesulfonate.

Suitable p38 kinase inhibitors include those disclosed in co-pending U.S. Patent Application No. 60/605,344, for example,
1-[5-tert-butyl-2-p-tolyl-2H-pyrazol-3-yl]-3-[4-(2-morpholin-4-ylethoxy)naphthalen-1-yl]urea;
1-[5-tert-butyl-2-p-tolyl-2H-pyrazol-3-yl]-3-[4-(2-(1-oxothiomorpholin-4-yl)ethoxy)naphthalen-1-yl]urea;
1-[5-tert-butyl-2-(2-methylpyridin-5-yl)-2H-pyrazol-3-yl]-3-[4-(2-pyridin-4-ylethoxy)naphthalen-1-yl]urea; and
1-[5-tert-butyl-2-(2-methoxypyridin-5-yl)-2H-pyrazol-3-yl]-3-[4-(2-morpholin-4-ylethoxy)naphthalen-1-yl]urea,
   and any pharmaceutically acceptable acid addition salts thereof.

Other suitable p38 MAP kinase inhibitors include, for example, compounds disclosed in co-pending U.S. Patent Application Nos. 60/598,621 and 60/630,517 and Indian Patent Application Nos. 1098/DEL/2005 and 211/DEL/2005, as well as:
1-[5-tert-butyl-2-methyl-2H-pyrazol-3-yl]-3-[4-(2-morpholin-4-ylethoxy)naphthalen-1-yl]urea;
4-[7-Oxo-8-(tetrahydro-pyran-4-yl)-6-o-tolyl-7,8-dihydro-pyrido[2,3-d]pyrimidin-2-ylamino]-piperidine-1-carboxylic acid tert-butyl ester;
Hydrochloride salt of 2-(Piperidin-4-ylamino)-8-(tetrahydro-pyran-4-yl)-6-o-tolyl-8H-pyrido[2,3-d]pyrimidin-7-one;
2-(1-Methanesulfonyl-piperidin-4-ylamino)-8-(tetrahydro-pyran-4-yl)-6-o-tolyl-H-pyrido[2,3-d]pyrimidin-7-one;
2-(1-Benzyl-piperidin-4-ylamino)-8-(tetrahydro-pyran-4-yl)-6-o-tolyl-8H-pyrido[2,3-d]pyrimidin-7-one;
2-(1-Methyl-piperidin-4-ylamino)-8-(tetrahydro-pyran-4-yl)-6-o-tolyl-8H-pyrido[2,3-d]pyrimidin-7-one;
2-(4-Methyl-piperazin-1-ylamino)-8-(tetrahydro-pyran-4-yl)-6-o-tolyl-5H-pyrido[2,3-d]pyrimidin-7-one;
4-[6-(2-Chloro-phenyl)-7-oxo-8-(tetrahydro-pyran-4-yl)-7,8-dihydro-pyrido[2,3-d]pyrimidin-2-ylamino]-piperidine-1-carboxylic acid tert-butyl ester;
2-(Piperidin-1-ylamino)-8-(tetrahydro-pyran-4-yl)-6-o-tolyl-8H-pyrido[2,3-d]pyrimidin-7-one;
2-Cyclobutylamino-8-(tetrahydro-pyran-4-yl)-6-o-tolyl-8H-pyrido[2,3-d]pyrimidin-7-one;
2-(1-Acetyl-piperidin-4-ylamino)-8-(tetrahydro-pyran-4-yl)-6-o-tolyl-5H-pyrido[2,3-d]pyrimidin-7-one;
2-(1-Benzoyl-piperidin-4-ylamino)-8-(tetrahydro-pyran-4-yl)-6-o-tolyl-8H-pyrido[2,3-d]pyrimidin-7-one;
2-(1-Benzoyl-piperidin-4-ylamino)-8-(tetrahydro-pyran-4-yl)-6-o-tolyl-8H-pyrido[2,3-d]pyrimidin-7-one;
4-[7-Oxo-8-(tetrahydro-pyran-4-yl)-6-o-tolyl-7,8-dihydro-pyrido[2,3-d]pyrimidin-2-ylamino]-piperidine-1-carboxylic acid (4-fluoro-phenyl)-amide;
2-(1-Ethanesulfonyl-piperidin-4-ylamino)-8-(tetrahydro-pyran-4-yl)-6-o-tolyl-5H-pyrido[2,3-d]pyrimidin-7-one;
4-[7-Oxo-8-(tetrahydro-pyran-4-yl)-6-o-tolyl-7,8-dihydro-pyrido[2,3-d]pyrimidin-2-ylamino]-piperidine-1-carbothioic acid (4-fluoro-phenyl)-amide;
4-[7-Oxo-8-(tetrahydro-pyran-4-yl)-6-o-tolyl-7,8-dihydro-pyrido[2,3-d]pyrimidin-2-ylamino]-piperidine-1-carboxylic acid (4-trifluoromethyl-phenyl)-amide;
2-[4-(Propane-2-sulfonyl)-piperazin-1-ylamino]-8-(tetrahydro-pyran-4-yl)-6-o-tolyl-8H-pyrido[2,3-d]pyrimidin-7-one;
4-[7-Oxo-8-(tetrahydro-pyran-4-yl)-6-o-tolyl-7,8-dihydro-pyrido[2,3-d]pyrimidin-2-ylamino]-piperazine-1-carboxylic acid propylamide;
4-[7-Oxo-8-(tetrahydro-pyran-4-yl)-6-o-tolyl-7,8-dihydro-pyrido[2,3-d]pyrimidin-2-ylamino]-piperazine-1-carboxylic acid ((R)-1,2-dimethyl-propyl)-amide;
4-[7-Oxo-8-(tetrahydro-pyran-4-yl)-6-o-tolyl-7,8-dihydro-pyrido[2,3-d]pyrimidin-2-ylamino]-piperazine-1-carboxylic acid cyclohexylamide;
4-[7-Oxo-8-(tetrahydro-pyran-4-yl)-6-o-tolyl-7,8-dihydro-pyrido[2,3-d]pyrimidin-2-ylamino]-piperazine-1-carboxylic acid (4-fluoro-phenyl)-amide; and
4-[7-Oxo-8-(tetrahydro-pyran-4-yl)-6-o-tolyl-7,8-dihydro-pyrido[2,3-d]pyrimidin-2-ylamino]-piperazine-1-carboxylic acid cyclopentyl methyl-amide, and
   any pharmaceutically acceptable acid addition salts thereof.

Pharmacologically acceptable acid addition salts include, for example, salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like.

Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-dibenzylethylenediamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine.

When a compound is basic, salts may be prepared from pharmaceutically acceptable non-toxic acids, including inorganic and organic acids, such as acetic, benzenesulfonic, benzoic, citric, ethanesulfonic, fumaric, gluconic, glutamic, hydrobromic, hydrochloric, isethionic, lactic, maleic, malic, mandelic, methanesulfonic, nitric, pantothenic, phosphoric, succinic, sulfuric, tartaric, and p-toluenesulfonic acid.

Pharmaceutical compositions described herein may be administered by following routes, for example, oral, topical, intravenous, intraarterial, intraperitoneal, intrathecal, intraventricular, intraurethral, intrasternal, intracranial, intramuscular, subcutaneous, intranasally, inhalation, rectally or vaginally.

Solid form preparations include powders, tablets, dispersible granules, capsules, cachets, suppositories, troches, patches, gel caps, magmas, lozenges, creams, pastes, plasters, lotions, discs, or ointments. Liquid form preparations include solutions suspensions, emulsions, syrups, elixirs, aerosols, inhalations, nasal spays or oral sprays.

Active compounds can be admixed under sterile condition with pharmaceutically acceptable carrier and any needed preservatives or buffer as may be required.

Pharmaceutical compositions for use in the methods described herein may be prepared by any of the methods of pharmacy, but all methods include the step of bringing into association one or more active compounds with one or more carriers or excipients. In general, pharmaceutical compositions are prepared by uniformly and intimately admixing the active compounds with one or more pharmaceutically acceptable liquid carriers or finely divided solid carriers or both, and then, if necessary, shaping the product into the desired form.

Commonly used carriers include one or more of corn starch, lactose, talc, calcium phosphate, calcium sulphate, calcium stearate, magnesium stearate, steane acid, sorbitol, microcrystalline cellulose, mannitol, gelatin, natural or synthetic gums, such as carboxymethylcellulose, methylcellulose, alginate, dextran, acacia gum, karaya gum, locust bean gum. Additionally, other excipients such as diluents, binders, lubricants, disintegrants, colors and flavoring agents may be employed. For example, a tablet may be prepared by compression or molding, optionally with one or more pharmaceutically acceptable excipient. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

In addition to the common dosage forms set out above, the therapeutically active ingredients may also be administered by controlled release means and/or delivery devices to provide the rate-controlled release of any one or more of the components or active ingredients to optimize the desired therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

The “polymeric matrix” serves essentially to modulate drug release kinetics and to stabilize metastable drug. Due to their versatility, polymers represent election material for matrix delivery systems. Polymeric matrices can be used in, for example, oral delivery, implantable systems, tissue engineering, DNA/RNA release, intelligent delivery systems and polymer conjugation.

The magnitude of a prophylactic or therapeutic dose of one or more compounds described herein in the acute or chronic prevention, treatment, or management of a disorder or condition will vary with the severity of the condition to be treated and the route of administration. The dose, and perhaps the dose frequency, will also vary according to the age, body weight, and response of the individual patient. Suitable total daily dose ranges can be readily determined by those skilled in the art.

The MRA and β2-agonists may be present in ratios from about 1:10 to 10:1. The MRA and β2-agonists may also be present in ratios of about 1:1, 2:1, 1:2, 1:3, 3:1, 1:5 and even 5:1.

The MRA and corticosteroids may be present in ratios from about 1:10 to 10:1. The MRA and corticosteroids may also be present in ratios of about 1:1, 2:1, 1:2, 1:3, 3:1, 1:5 and even 5:1.

The MRA and p38 MAP kinase inhibitors may be present in ratios from about 1:10 to 10:1. The MRA and p38 MAP kinase inhibitors may also be present in ratios of about 1:1, 2:1, 1:2, 1:3, 3:1, 1:5 and even 5:1.

The MRA and PDE-IV inhibitors may be present in ratios from about 1:10 to 10:1. The MRA and PDE-IV inhibitors may also be present in ratios of about 1:1, 2:1, 1:2, 1:3, 3:1, 1:5 and even 5:1.

Suitable dosage amounts can be determined using small dosages that are less than the optimum dose. Such small dosages can be increased in small increments until the optimum effect is reached. Dosage amounts may be divided and administered as divided doses if desired.

The present invention also provides for methods of treating or preventing autoimmune, inflammatory, or allergic disorders. The method comprises administering to a mammal in need thereof a pharmaceutical composition comprising therapeutically effective amounts of one or more MRA of Formulae I, II, or III described herein, and at least one additional active ingredients selected from one or more β2-agonists, p38 MAP kinase, PDE-IV inhibitors, corticosteroids or a mixture thereof and optionally one or more pharmaceutically acceptable carriers, excipients or diluents.

In one embodiment, there is provided methods for treating or preventing autoimmune and/or inflammatory/allergic diseases or disorders comprising administering one or more compounds of pharmaceutical compositions described herein. Such autoimmune and/or inflammatory/allergic diseases or disorder include, for example, respiratory disorder, asthma, chronic bronchitis, chronic obstructive pulmonary disease, whooping cough, eosinophilic granuloma, psoriasis and other benign or malignant proliferative skin diseases, eczema, inflammatory bowel disease, endotoxic shock, anaphylactic shock, laminitis in horses, septic shock, ulcerative colitis, crohn's disease, reperfusion injury of the myocardium and brain, inflammatory arthritis, periodontitis, chronic glomerulonephritis, atopic dermatitis, urticaria, adult respiratory distress syndrome, infant respiratory distress syndrome, transplant rejection, rhinitis, pruritus, diabetes insipidus, eye diseases, allergic rhinitis, allergic conjunctivitis, vernal conjunctivitis, arterial restenosis, ortheroselerosis, atherosclerosis, neurogenic inflammation, pain, cough, rheumatoid arthritis, osteoporosis, osteoarthritis, inflammation, ankylosing spondylitis, transplant rejection, graft versus host disease, hypersecretion of gastric acid, bacterial, fungal induced sepsis, viral induced sepsis, fungal induced septic shock, viral induced septic shock, inflammation-mediated chronic tissue degeneration, cytokine-mediated chronic tissue degeneration, osteoarthritis, cancer, cachexia, muscle wasting, depression memory impairment, tumor growth, cancerous invasion of normal tissues Hashimoto's thyroiditis (underactive thyroid), Graves' disease (overactive thyroid), Lupus and acquired immuno deficiency syndrome.

In some embodiments, methods of treating or preventing autoimmune, inflammatory or allergic disorders include concurrent or sequential administration to a mammal in need thereof, a) a pharmaceutical composition comprising a therapeutically effective amount of one or more compounds described, and one or more pharmaceutically acceptable carriers, excipients or diluents; and b) one or more pharmaceutical compositions comprising therapeutically effective amounts of at least one active ingredient selected from one or more of β2-agonists, one or more p38 MAP kinase inhibitors, one or more PDE-IV inhibitors, one or more corticosteroids and one or more pharmaceutically acceptable carriers, excipients or diluents.

In some embodiments, methods of treating or preventing autoimmune, inflammatory or allergic disorders include concurrent or sequential administration to a mammal in need thereof: a) a pharmaceutical composition comprising a therapeutically effective amount of one or more compounds described herein, and one or more pharmaceutically acceptable carriers, excipients or diluents; and b) one or more pharmaceutical compositions comprising therapeutically effective amounts of at least one active ingredient selected from one or more of anticholinergics, one or more dopamine agonists, one or more antiallergics, one or more PAF antagonists, one or more leukotriene antagonists, one or more EGFR kinase inhibitors, one or more additional muscarinic receptor antagonists, or combinations thereof, and one or more pharmaceutically acceptable carriers, excipients or diluents.

MRA compounds described herein may be used on their own or in conjunction with other active MRA compounds known in the art. MRA compounds described herein may also be used in combination with other pharmaceutically active substances. These may be, for example, one or more anticholinergics, dopamine agonists, antiallergics, PAF antagonists, leukotriene antagonists, EGFR kinase inhibitors, MRAs, or mixtures thereof.

Suitable anticholinergics include, but are not limited to, anticholinergics known in the art, as well as tiotropium salts, ipratropium salts, oxitropium salts, salts of one or more compounds disclosed in WO 02/32899; tropenol N-methyl-2,2-diphenylpropionate, scopine N-methyl-2,2-diphenylpropionate, scopine N-methyl-2-fluoro-2,2-diphenylacetate and tropenol N-methyl-2-fluoro-2,2-diphenylacetate; as well as salts of the compounds disclosed in WO 02/32898; tropenol N-methyl-3,3′,4,4′-tetrafluorobenzilate, scopine N-methyl-3,3′,4,4′-tetrafluorobenzilate, scopine N-methyl-4,4′-dichlorobenzilate, scopine N-methyl-4,4′-difluorobenzilate, tropenol N-methyl-3,3′-difluorobenzilate, scopine N-methyl-3,3′-difluorobenzilate, and tropenol N-ethyl-4,4′-difluorobenzilate, optionally in hydrate and solvate forms thereof. Salts include abovementioned cations, and anions including, for example, chloride, bromide, and methanesulfonate. In some embodiments, salts include bromide or methanesulfonate salts of such compounds.

Suitable anticholinergics include, but are not limited to, anticholinergics known in the art, as well as one or more of tiotropium bromide, ipratropium bromide, oxitropium bromide, tropenol 2,2-diphenylpropionate methobromide, scopine 2,2-diphenylpropionate methobromide, scopine 2-fluoro-2,2-diphenylacetate methobromide, tropenol 2-fluoro-2,2-diphenylacetate methobromide, tropenol 3,3′,4,4′-tetrafluorobenzilate methobromide, scopine 3,3′,4,4′-tetrafluorobenzilate methobromide; scopine 4,4′-dichlorobenzilate methobromide, scopine 4,4′-difluorobenzilate methobromide, tropenol 3,3′-difluorobenzilate methobromide, scopine 3,3′-difluorobenzilate methobromide, tropenol 4,4′-difluorobenzilate ethylbromide or mixtures thereof. In some embodiments, anticholinergics include one or more of tiotropium bromide, ipratropium bromide, tropenol 2,2-diphenylpropionate methobromide, scopine 2,2-diphenylpropionate methobromide, scopine 2-fluoro-2,2-diphenylacetate methobromide, tropenol 2-fluoro-2,2-diphenylacetate methobromide or mixtures thereof.

Suitable corticosteroids include, but are not limited to, corticosteroids known in the art, as well as one or more of flunisolide, beclomethasone, triamcinolone, budesonide, fluticasone, mometasone, ciclesonide, rofleponide, GW 215864, KSR 592, ST-126, dexamethasone or mixtures thereof. In some embodiments, the corticosteroids can be selected from one or more of flunisolide, beclomethasone, triamcinolone, budesonide, fluticasone, mometasone, ciclesonide, dexamethasone or mixtures thereof; from one or more of budesonide, fluticasone, mometasone, ciclesonide or mixtures thereof; and fluticasone. Suitable corticosteroids include salts or derivatives thereof, including, for example, sodium salts, sulfobenzoates, phosphates, isonicotinates, acetates, propionates, dihydrogen phosphates, palmitates, pivalates, or furoates. In some embodiments, corticosteroids are in the form of their hydrates.

Suitable PDE-IV inhibitors include, but are not limited to, PDE-IV inhibitors known in the art, as well as one or more compounds disclosed in WO 2005/021515 and co-pending Indian Patent Application No. 303/DEL/2005, compounds disclosed hereinabove; as well as one or more of enprofylline, roflumilast, aniflo, Bay-198004, CP-325, 366, BY343, D-4396 (Sch-351591), V-11294A, Z-15370, AWD-12-281; or mixtures thereof. In some embodiments, suitable PDE-IV inhibitors can be selected from one or more of enprofylline, roflumilast, ariflo, Z15370, AWD-12-281, compounds disclosed in WO 2005/021515 and co-pending Indian Patent Application No. 303/DEL/2005 or mixtures thereof. In other embodiments, the suitable PDE-IV inhibitor can be AWD-12-281. PDE-IV inhibitors can include any pharmaceutically acceptable acid addition salts thereof, which may exist. Pharmaceutically acceptable salts can be selected from salts of hydrochloric acid, hydrobromic acid, sulfuric acid, phosphoric acid, methanesulfonic acid, acetic acid, fumaric acid, succinic acid, lactic acid, citric acid, tartaric acid, or maleic acid. In some embodiments, the salts can be selected from acetate, hydrochloride, hydrobromide, sulfate, phosphate, and methanesulfonate.

Suitable dopamine agonists include, but are not limited to, dopamine agonists known in the art, as well as one or more of bromocriptine, cabergolin, α-dihydroergocryptine, lisuride, pergolide, pramipexol, roxindole, ropinirole, talipexole, terguride, viozan or mixtures thereof. In some embodiments, suitable dopamine agonists can be selected from one or more of pramipexol, talipexole, viozan or mixtures thereof. Dopamine agonists include pharmaceutically acceptable acid addition salts and hydrates thereof, which may exist. Pharmaceutically acceptable acid addition salts can be selected from salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid.

Suitable antiallergic agents include, but are not limited to, antiallergic agents known in the art, as well as, one or more of epinastine, cetirizine, azelastine, fexofenadine, levocabastine, loratadine, mizolastine, ketotifene, emedastine, dimethindene, clemastine, bamipine, hexachloropheniramine, pheniramine, doxylamine, chlorophenoxamine, dimenhydrinate, diphenhydramine, promethazine, ebastine, desloratadine, meclizine or mixtures thereof. In some embodiments, suitable antiallergic agents can be selected from one or more of epinastine, cetirizine, azelastine, fexofenadine, levocabastine, loratadine, ebastine, desloratadine, mizolastine or mixtures thereof; as well as, epinastine, desloratadine or mixtures thereof. Antiallergic agents include pharmaceutically acceptable acid addition salts thereof, which may exist.

Suitable PAF antagonists include, but are not limited to, PAF antagonists known in the art, as well as one or more of 4-(2-chlorophenyl)-9-methyl-2-[3-(4-morpholinyl)-3-propanol-yl]-6H-thieno[3,2-f][1,2,4]triazolo[4,3-α][1,4]diazepine, 6-(2-chlorophenyl)-8,9-dihydro-1-methyl-8-[(4-morpholinyl)carbonyl]-4H,7H-cyclopenta[4.5]thieno[3,2-f][1,2,4]triazolo[4,3-a][1,4]diazepine or mixtures thereof.

Suitable EGFR kinase inhibitors include, but are not limited to, EGFR kinase inhibitors known in the art, as well as one or more of 4-[(3-chloro-4-fluorophenyl)amino]-7-(2-{4-[(S)-(2-oxotetrahydrofuran-5-yl)carbonyl]piperazin-1-yl}-ethoxy)-6-[(vinylcarbonyl)amino]quinazoline, 4-[(3-chloro-4-fluorophenyl)amino]-7-[4-((S)-6-methyl-2-oxomorpholin-4-yl)butyloxy]-6-[(vinylcarbonyl)amino]quinazoline, 4-[(3-chloro-4-fluorophenyl)amino]-7-[4-((R)-6-methyl-2-oxomorpholin-4-yl)butyloxy]-6-[(vinylcarbonyl)amino]quinazoline, 4-[(3-chloro-4-fluorophenyl)amino]-7-[2-((S)-6-methyl-2-oxomorpholin-4-yl)ethoxy]-6-[(vinylcarbonyl)amino]quinazoline, 4-[(3-chloro-4-fluorophenyl)amino]-6-[(4-{N-[2-(ethoxycarbonyl)ethyl]-N-[(ethoxycarbonyl)methyl]-amino}-1-oxo-2-buten-1-yl)amino]-7-cyclopropylmethoxyquinazoline, 4-[(R)-(1-phenylethyl)amino]-6-{[4-(morpholin-4-yl)-1-oxo-2-buten-1-yl]amino}-7-cyclopropylmethoxyquinazoline, 4-[(3-chloro-4-fluorophenyl)amino]-6-[3-(morpholin-4-yl)propyloxy]-7-methoxyquinazoline or mixtures thereof. EGFR kinase inhibitors include pharmaceutically acceptable acid addition salts thereof, which may exist. Pharmaceutically acceptable acid addition salts include, for example, salts of hydrochloric acid, hydrobromic acid, sulfuric acid, phosphoric acid, methanesulfonic acid, acetic acid, fumaric acid, succinic acid, lactic acid, citric acid, tartaric acid, or maleic acid. For example, salts of EGFR kinase inhibitors can be selected from salts of acetic acid, hydrochloric acid, hydrobromic acid, sulfuric acid, phosphoric acid, and methanesulfonic acid.

Suitable p38 kinase inhibitors include, but are not limited to, p38 kinase inhibitors known in the art, as well as one or more of 1-[5-tert-butyl-2-p-tolyl-2H-pyrazol-3-yl]-3-[4-(2-morpholin-4-ylethoxy)naphthalen-1-yl]urea; 1-[5-tert-butyl-2-p-tolyl-2H-pyrazol-3-yl]-3-[4-(2-(1-oxothiomorpholin-4-yl)ethoxy)naphthalen-1-yl]urea; 1-[5-tert-butyl-2-(2-methylpyridin-5-yl)-2H-pyrazol-3-yl]-3-[4-(2-pyridin-4-ylethoxy)naphthalen-1-yl]urea; 1-[5-tert-butyl-2-(2-methoxypyridin-5-yl)-2H-pyrazol-3-yl]-3-[4-(2-morpholin-4-ylethoxy)naphthalen-1-yl]urea; 1-[5-tert-butyl-2-methyl-2H-pyrazol-3-yl]-3-[4-(2-morpholin-4-ylethoxy)naphthalen-1-yl]urea or mixtures thereof (disclosed in co-pending U.S. Patent Application No. 60/605,344); 4-[7-Oxo-8-(tetrahydro-pyran-4-yl)-6-o-tolyl-7,8-dihydro-pyrido[2,3-d]pyrimidin-2-ylamino]-piperidine-1-carboxylic acid tert-butyl ester; Hydrochloride salt of 2-(Piperidin-4-ylamino)-8-(tetrahydro-pyran-4-yl)-6-o-tolyl-5H-pyrido[2,3-d]pyrimidin-7-one; 2-(1-M ethanesulfonyl-piperidin-4-ylamino)-8-(tetrahydro-pyran-4-yl)-6-o-tolyl-8H-pyrido[2,3-d]pyrimidin-7-one; 2-(1-Benzyl-piperidin-4-ylamino)-8-(tetrahydro-pyran-4-yl)-6-o-tolyl-8H-pyrido[2,3-d]pyrimidin-7-one; 2-(1-Methyl-piperidin-4-ylamino)-8-(tetrahydro-pyran-4-yl)-6-o-tolyl-8H-pyrido[2,3-d]pyrimidin-7-one; 2-(4-Methyl-piperazin-1-ylamino)-8-(tetrahydropyran-4-yl)-6-o-tolyl-8H-pyrido[2,3-d]pyrimidin-7-one; 4-[6-(2-Chloro-phenyl)-7-oxo-8-(tetrahydro-pyran-4-yl)-7,8-dihydro-pyrido[2,3-d]pyrimidin-2-ylamino]-piperidine-1-carboxylic acid tert-butyl ester; 2-(Piperidin-1-ylamino)-8-(tetrahydro-pyran-4-yl)-6-o-tolyl-8H-pyrido[2,3-d]pyrimidin-7-one; 2-Cyclobutylamino-8-(tetrahydro-pyran-4-yl)-6-o-tolyl-8H-pyrido[2,3-d]pyrimidin-7-one; 2-(1-Acetyl-piperidin-4-ylamino)-8-(tetrahydro-pyran-4-yl)-6-o-tolyl-8H-pyrido[2,3-d]pyrimidin-7-one; 2-(1-Benzoyl-piperidin-4-ylamino)-8-(tetrahydro-pyran-4-yl)-6-o-tolyl-8H-pyrido[2,3-d]pyrimidin-7-one; 2-(1-Benzoyl-piperidin-4-ylamino)-8-(tetrahydro-pyran-4-yl)-6-o-tolyl-8H-pyrido[2,3-d]pyrimidin-7-one; 4-[7-Oxo-8-(tetrahydro-pyran-4-yl)-6-o-tolyl-7,8-dihydro-pyrido[2,3-d]pyrimidin-2-ylamino]-piperidine-1-carboxylic acid (4-fluoro-phenyl)-amide; 2-(1-Ethanesulfonyl-piperidin-4-ylamino)-8-(tetrahydro-pyran-4-yl)-6-o-tolyl-8H-pyrido[2,3-d]pyrimidin-7-one; 4-[7-Oxo-8-(tetrahydro-pyran-4-yl)-6-o-tolyl-7,8-dihydro-pyrido[2,3-d]pyrimidin-2-ylamino]-piperidine-1-carbothioic acid (4-fluoro-phenyl)-amide; 4-[7-Oxo-8-(tetrahydro-pyran-4-yl)-6-o-tolyl-7,8-dihydro-pyrido[2,3-d]pyrimidin-2-ylamino -piperidine-1-carboxylic acid (4-trifluoromethyl-phenyl)-amide; 2-[4-(Propane-2-sulfonyl)-piperazin-1-ylamino]-8-(tetrahydro-pyran-4-yl)-6-o-tolyl-5H-pyrido[2,3-d]pyrimidin-7-one; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid propylamide; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid ((R)-1,2-dimethyl)-propyl)-amide; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid cyclohexylamide; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid (4-fluoro-phenyl)-amide; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid cyclopentyl methyl-amide; one or more compounds disclosed in co-pending U.S. Patent Application Nos. 60/598621 and 60/630,517 and Indian Patent Application Nos. 1098/DEL/2005 and 211/DEL/2005; or mixtures thereof p38 kinase inhibitors include pharmaceutically acceptable acid addition salts thereof which may exist. Pharmaceutically acceptable salts can be selected from salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid.

Suitable muscarinic receptor antagonists include substances that directly or indirectly block activation of muscarinic cholinergic receptors. Examples include, but are not limited to, quaternary amines (e.g., methantheline, ipratropium, propantheline), tertiary amines (e.g., dicyclomine, scopolamine) and tricyclic amines (e.g., telenzepine).

Other suitable muscarinic receptor antagonists include benztropine (commercially available as COGENTIN from Merck), hexahydro-sila-difenidol hydrochloride (HHSID hydrochloride disclosed in Lambrecht et al., Trends in Pharmacol. Sci., 10(Suppl):60 (1989); (+/−)-3-quinuclidinyl xanthene-9-carboxylate hemioxalate (QNX-hemioxalate; Birdsall et al., Trends in Pharmacol. Sci., 4:459 (1983); telenzepine dihydrochloride (Coruzzi et al., Arch. Int. Pharmacodyn. Ther., 302:232 (1989); and Kawashima et al., Gen. Pharmacol., 21:17 (1990)), and atropine.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

### Biological Assay Method

### Example 1

### In-Vitro Functional Assay to Evaluate Efficacy of “MRA” in Combination with “PDE-IV Inhibitors”

### Animals and Anaesthesia:

Guinea Pigs (400-600 gm) were procured and trachea was removed under anesthesia (sodium pentobarbital, 300 mg/kg i.p) and immediately kept in ice-cold Krebs Henseleit buffer. Indomethacin (10 uM) was present throughout the KH buffer to prevent the formation of bronchoactive prostanoids.

### Trachea Experiments:

The tissue of adherent fascia was removed and cut into strips of equal size (with approx. 4-5 tracheal rings in each strip). The epithelium was removed by careful rubbing, minimizing damage to the smooth muscle. The trachea was opened along the mid-dorsal surface with the smooth muscle band intact and a series of transverse cuts made from alternate sides so that they do not transect the preparation completely. Opposite ends of the cut rings were tied with the help of a thread. The tissue was mounted in isolated tissue baths containing 10 ml Krebs Henseleit buffer maintained at 37° C. and bubbled with carbogen, at a basal tension of 1 gm. The buffer was changed 4-5 times for about an hour. Equilibration of the tissue was done for 1 hr for stabilization. After 1 hr, the tissue was challenged with 1 μM carbachol. This was repeated after every 2-3 washes till two similar consecutive responses were obtained. At the end of stabilization, the tissues were incubated with suboptimal dose of MRA/Vehicle for 20 minutes prior to contraction of the tissues with 1 μM carbachol. The relaxant activity of the PDE-IV inhibitor [10⁻⁹M to 10⁻⁴ M] on the stabilized developed tension/response was subsequently assessed. The contractile response of tissues was recorded either on Powerlab data acquisition system or on Grass polygraph (Model 7). The relaxation was expressed as percentage of maximum carbachol response and EC₂₅ was calculated as the concentration producing 25% of the maximum relaxation to 1 μM carbachol. The percent relaxation between the treated and control tissues were compared using non-parametric unpaired t-test. A p value of <0.05 is considered to be statistically significant.

Preincubation of tissues with C No. 66 at 1 nM before contraction with carbachol potentiated the subsequent relaxant activity of C No. 124aa, roflumilast and rolipram. This was apparent from the slight but significant shift in the −log[EC₂₅] value from 4.40 to 5.53 for C No. 124aa (p<0.05) & from 4.46 to 6.25 for roflumilast (p<0.01) in the presence of C No. 66. There was no significant potentiation of the response for rolipram in the presence of C No. 66 (p>0.05)

TABLE 1
Potency of the compounds for relaxing carbachol
precontracted guinea-pig isolated trachea

| | Tension(gm) | | | |
|---|---|---|---|---|
| | Before | After | | |
| | carbachol | carbachol | | |
| Treatment | challenge | challenge | - Log[EC₂₅] | EC₂₅(μM) |
| C No. 124aa (n = 3) | 1.84 ± 0.32 | 1.99 ± 0.40 | 4.40 | 41.8 |
| C No. 66 (1 nM) + C No. | 2.43 ± 0.38 | 2.25 ± 0.19 | 5.53 | 9.8* |
| 124aa (n = 3) | | | | |
| Rolipram (n = 2) | 1.24 ± 0.04 | 1.16 ± 0.30 | 5.25 | 7.6 |
| C No. 66 (1 nM) + Rolipram | 1.15 ± 0.23 | 1.23 ± 0.29 | 6.00 | 1.1^{ns} |
| (n = 2) | | | | |
| Roflumilast | 1.38 ± 0.22 | 1.57 ± 0.22 | 4.46 | 44.2 |
| (n = 5) | | | | |
| C No. 66 1 nM) + Roflumilast | 1.39 ± 0.32 | 1.33 ± 0.30 | 6.25 | 0.66^{@} |
| (n = 2) | | | | |

n: number of experiments;
*(p < 0.05) vs 14016;
^{ns}(p > 0.05) vs Rolipram;
^{@}(p < 0.01) vs Roflumilast
C No. 66 and C No. 124aa refers to Compound No. 66 and 124aa, respectively.

### Example 2

### In-Vivo Assay to Evaluate Efficacy of MRA in Combination with PDE-IV Inhibitors

### Drug Treatment:

MRA (1 ng/kg to 1 mg/kg) and PDE-IV inhibitor (1 ng/kg to 1 mg/kg) were instilled intratracheally under anesthesia either alone or in combination.

### Method:

Wistar rats weighing 200±20 gm were used in the study. Rats had free access to food and water. On the day of experiment, animals were exposed to lipopolysaccharide (LPS, 100 μg/ml) for 40 min. One group of vehicle treated rats was exposed to phosphate buffered saline (PBS) for 40 min. Two hours after LPS/PBS exposure, animals were placed inside a whole body plethysmograph (Buxco Electronics, USA) and exposed to PBS or increasing concentration of acetylcholine (1, 6, 12, 24, 48 and 96 mg/ml) aerosol until Penh values (index of airway resistance) of rats attained 2 times the value (PC-100) seen with PBS alone. The respiratory parameters were recorded online using Biosystem XA software, (Buxco Electronics, USA). Penh, at any chosen dose of acetylcholine was, expressed as percent of PBS response and the using a nonlinear regression analysis PC100 (2 folds of PBS value) values computed.

A synergistic effect was observed with the combination of muscarinic receptor antagonist (MRA) with PDE 4 inhibitor which can be seen from below mentioned graphs.

### Example 3

### In-Vivo Assay to Evaluate Efficacy of MRA in Combination with Corticosteroids

### Ovalbumin Induced Early Phase Bronchoconstriction and Airway Inflammation:

Guinea pigs are sensitised on days 0, 7 and 14 with 50-μg ovalbumin and 10 mg aluminium hydroxide injected intraperitoneally. On days 19 and 20 guinea pigs are exposed to 0.1% w v⁻¹ ovalbumin or PBS for 10 min, and with 1% ovalbumin for 30 min on day 21. Guinea pigs are treated with test compound or standard or vehicle once daily from day 19 and continued for 4 days.

### Ovalbumin Induced Early Phase Bronchoconstriction

On day 21, after drug or vehicle administration, basal respiratory parameters are recorded using Whole body Plethysmograph (Biosystem XA software, Buxco Electronics, USA) followed by challenge with 1% ovalbumin/PBS for 10 min duration. For recording basal respiratory parameters, 10 consecutive 1 min readings are averaged. Each 1 min. reading represents an average of each breadth taken in that 60 sec duration. Following PBS/Ovalbumin challenge data is recorded for 120 min, which represented hundred and twenty recordings one min apart. Each 1 min recording is an average of all the breath in 1 min. PenH, at any chosen time point post challenge is expressed as percent of basal response. These values are plotted against time using Graphpad prism software (GraphPad Software Inc, USA) and Area Under the Curve (AUC) is computed. Percent inhibition is computed using the following formula.

$$Percent Inhibition = \frac{A U {C}_{OVA} - A U {C}_{TEST}}{A U {C}_{OVA} - A U {C}_{P B S}} \times 100$$

### Where,

AUC_{OVA}=AUC in vehicle treated group challenged with ovalbumin
AUC_{TEST}=AUC in group treated with a given dose of test compound
AUC_{PBS}=AUC in vehicle treated group challenged with PBS

### Ovalbumin Induced Airway Inflammation

24 hrs after the final ovalbumin challenge BAL is performed using Hank's balanced salt solution (HBSS). Collected lavage fluid is centrifuged at 3000 rpm for 5 min, at 4° C. Pellet is collected and resuspended in 1 ml HBSS. Total leukocyte count is performed in the resuspended sample. A portion of suspension is cytocentrifuged and stained with Leishmann's stain for differential leukocyte count. Total leukocyte and eosinophil count are expressed as cell count (millions cells ml⁻¹ of BAL). Eosinophil is also expressed as percent of total leukocyte count. % inhibition is computed using the following formula.

$$\% Inhibition = \frac{{Eos}_{OVA} - {Eos}_{TEST}}{{Eos}_{OVA} - {Eos}_{CON}} \times 100$$

### Where,

Eos_{OVA}=Percentage of eosinophil in vehicle treated group challenged with ovalbumin
Eos_{TEST}=Percentage of eosinophil in group treated with a given dose of test compound
Fos_{CON}=Percentage of eosinophil in vehicle treated group challenged with PBS.

### Example 4

### In-Vivo Assay to Evaluate Efficacy of “MRA” in Combination with p38 MAP Kinase Inhibitors

### Lipopolysaccharide (LPS) Induced Airway Hyperreactivity (AHR) and Neutrophilia:

### Drug Treatment:

MRA (1 ng/kg to 1 mg/kg) and p38 MAP kinase inhibitor (1 ng/kg to 1 mg/kg) are instilled intratracheally under anesthesia either alone or in combination.

### Method:

Male wistar rats weighing 200±20 gm are used in the study. Rats have free access to food and water. On the day of experiment, animals are exposed to lipopolysaccharide (LPS, 100 μg/ml) for 40 in. One group of vehicle treated rats is exposed to phosphate buffered saline (PBS) for 40 min. Two hours after LPS/PBS exposure, animals are placed inside a whole body plethysmograph (Buxco Electronics, USA) and exposed to PBS or increasing acetylcholine (1, 6, 12, 24, 48 and 96 mg/ml) aerosol until Penh values (index of airway resistance) of rats attained 2 times the value (PC-100) seen with PBS alone. The respiratory parameters are recorded online using Biosystem XA software, (Buxco Electronics, USA). Penh, at any chosen dose of acetylcholine is, expressed as percent of PBS response and the using a nonlinear regression analysis PC100 (2 folds of PBS value) values are computed. Percent inhibition is computed using the following formula.

$$\% Inhibition = \frac{PC {100}_{L P S} - PC {100}_{TEST}}{PC {100}_{L P S} - PC {100}_{P B S}} \times 100$$

### Where,

PC100_{LPS}=PC100 in vehicle treated group challenged group with LPS
PC100_{TEST}=PC100 in group treated with a given dose of test compound
PC100_{PBS}=PC100 in vehicle treated group challenged with PBS

Immediately after the airway hyperreactivity response is recorded, animals are sacrificed and bronchioalveolar lavage (BAL) is performed. Collected lavage fluid is centrifuged at 3000 rpm for 5 min, at 4° C. Pellet is collected and resuspended in 1 ml HBSS. Total leukocyte count is performed in the resuspended sample. A portion of suspension is cytocentrifuged and stained with Leishmann's stain for differential leukocyte count. Total leukocyte and Neutrophil counts are expressed as cell count (millions cells ml⁻¹ of BAL). Percent inhibition is computed using the following formula.

$$\% Inhibition = \frac{{NC}_{L P S} - {NC}_{TEST}}{{NC}_{L P S} - {NC}_{P B S}} \times 100$$

### Where,

NC_{LPS}=Percentage of neutrophil in vehicle treated group challenged with LPS
NC_{TEST}=Percentage of neutrophil in group treated with a given dose of test compound
NC_{PBS}=Percentage of neutrophil in vehicle treated group challenged with PBS
The percent inhibition data is used to compute ED₅₀ vales using Graph Pad Prism software (Graphpad Software Inc., USA).

### Example 5

### In-Vivo Assay to Evaluate Efficacy of “MRA” in Combination with β2-Agonists

### Drug Treatment:

MRA (1 ng/kg to 1 mg/kg) and long acting β2 agonist are instilled intratracheally under anesthesia either alone or in combination.

### Method

Wistar rats (250-350 gm) or balb/C mice (20-30 gm) are placed in body box of a whole body plethysmograph (Buxco Electronics, USA) to induce bronchoconstriction. Animals are allowed to acclimatise in the body box and are given successive challenges, each of 2 min duration, with PBS (vehicle for acetylcholine) or acetylcholine (i.e. 24, 48, 96, 144, 384, and 768 mg/ml). The respiratory parameters are recorded online using Biosystem XA software, (Buxco Electronics, USA) for 3 min. A gap of 2 min is allowed for the animals to recover and then challenged with the next higher dose of acetylcholine (ACh). This step is repeated until Penh of rats attained 2 times the value (PC-100) seen with PBS challenge. Following PBS/ACh challenge, Penh values (index of airway resistance) in each rat/mice is obtained in the presence of PBS and different doses of ACh. Penh, at any chosen dose of ACh is, expressed as percent of PBS response. The Penh values thus calculated are fed into Graph Pad Prism software (Graphpad Software Inc., USA) and using a nonlinear regression analysis PC100 (2 folds of PBS value) values are computed. Percent inhibition is computed using the following formula.

$$Percent Inhibition = \frac{PC {100}_{TEST} - PC {100}_{CON}}{768 - PC {100}_{CON}} \times 100$$

### Where,

PC100_{CON}=PC100 in vehicle treated group
PC100_{TEST}=PC100 in group treated with a given dose of test compound
768=is the maximum amount of acetylcholine used.

1. A pharmaceutical composition comprising one or more muscarinic receptor antagonists (“MRA”), and at least one additional active ingredients selected from one or more β2-agonists, p38 MAP kinase inhibitors, PDE-IV inhibitors, corticosteroids, anticholinergics, dopamine agonists, antiallergics, PAF antagonists, leukotriene antagonists, EGFR kinase inhibitors, different muscarinic receptor antagonists or a mixture thereof, wherein the MRA is one or more compounds having the structures of Formula I, II, or III, wherein:
a. Formula I is: 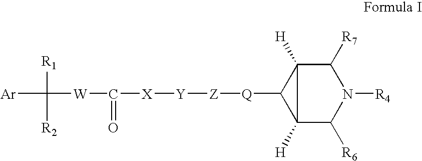
or a pharmaceutically acceptable salt, pharmaceutically acceptable solvate, ester, enantiomer, diastereomer, N-oxide, polymorphs, prodrugs or metabolite thereof, wherein
Ar represents an aryl or a heteroaryl ring having 1-2 heteroatoms independently selected from oxygen, sulphur or nitrogen, wherein
the aryl or heteroaryl ring may be unsubstituted or substituted by one to three substituents independently selected from lower alkyl (C₁-C₄), lower perhalo alkyl (C₁-C₄), cyano, hydroxy, nitro, lower alkoxy (C₁-C₄), lower perhalo alkoxy (C₁-C₄), unsubstituted amino, N-lower alkyl (C₁-C₄), N-aryl amino, amino carbonyl, N-lower alkyl (C₁-C₄) or N-aryl amino carbonyl;
R₁ represents hydrogen, hydroxy, hydroxy methyl, substituted or unsubstituted amino, alkoxy, carbamoyl or halogen (e.g., fluorine, chlorine, bromine and iodine);
R₂ represents alkyl, (C₃-C₇) cycloalkyl ring, (C₃-C₇) cycloalkenyl ring, aryl, heterocyclic ring, or heteroaryl ring, wherein
the heterocyclic ring or heteroaryl ring may have 1 to 2 heteroatoms independently selected from oxygen, sulphur or nitrogen, and
the aryl or heteroaryl ring may be unsubstituted or substituted by one to three substituents independently selected from lower alkyl (C₁-C₄), lower perhalo alkyl (C₁-C₄), cyano, hydroxy, nitro, lower alkoxycarbonyl, halogen, lower alkoxy (C₁-C₄), lower perhalo alkoxy (C₁-C₄), unsubstituted amino, N-lower alkyl (C₁-C₄) or N-aryl amino, amino carbonyl, N-lower alkyl (C₁-C₄) or N-aryl amino carbonyl;
W represents (CH₂)ₚ, wherein p represents 0 to 1;
X represents oxygen, sulphur, —NR or no atom (i.e., a bond), wherein
R represents hydrogen or (C₁₋₆) alkyl;
Y represents CHR₅CO or (CH₂)_{q}, wherein
R₅ represents hydrogen or methyl, and
q represents 0 to 4;
Z represents oxygen, sulphur, or NR₁₀, wherein
R₁₀ represents hydrogen, or C₁₋₆ alkyl;
Q represents (CH₂)ₙ, CHR₉ or CH₂CHR₉, wherein
n represents 0 to 4,
R₈ represents H, OH, C₁₋₆, alkyl, C₁₋₆ alkenyωl, or C₁₋₆ alkoxy, and
R₉ represents H, OH, lower alkyl (C₁-C₄) or lower alkoxy (C₁-C₄);
R₄ and R₇ are independently selected from H, CH₃, COOH, CONH₂, NH₂ or CH₂NH₂; and
R₄ represents hydrogen or C₁-C₁₅ saturated or unsaturated aliphatic hydrocarbon group, wherein
1 to 6 hydrogen atoms of C₁-C₁₅ saturated or unsaturated aliphatic hydrocarbon group may be substituted with a group independently selected from halogen, arylalkyl, arylalkenyl, heteroarylalkyl or heteroarylalkenyl, wherein
heteroarylalkyl or heteroarylalkenyl may have 1 to 2 heteroatoms independently selected nitrogen, oxygen or sulphur, and any 1 to 3 hydrogen atoms on the ring of arylalkyl, arylalkenyl, heteroarylalkenyl may be optionally substituted with lower alkyl (C₁-C₄), lower perhalo alkyl (C₁-C₄), cyano, hydroxyl, nitro, lower alkoxycarbonyl, halogen, lower alkoxy (C₁-C₄), lower perhaloalkoxy (C₁-C₄), unsubstituted amino, N-lower alkylamino (C₁-C₄), or N-lower alkylamino carbonyl (C₁-C₄);
b. Formula II is: 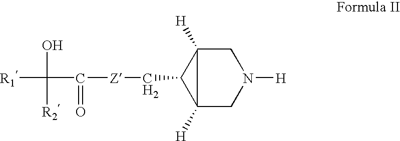
or a pharmaceutically acceptable salt, pharmaceutically acceptable solvate, ester, enantiomer, diastereomer, N-oxide, polymorph or metabolite thereof, wherein
R₁′ and R₂′ are independently selected from C₁-C₆ alkyl, C₃-C₇ cycloalkyl or phenyl, wherein phenyl is optionally substituted with one or more groups independently selected from C₁-C₃ alkyl, C₁-C₃ alkoxy or halogen; and
Z′ represents oxygen or NR₃, wherein
R₃ represents hydrogen or C₁-C₃ alkyl;
c. Formula III is, 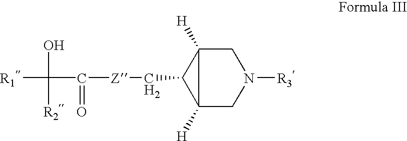
or a pharmaceutically acceptable salt, pharmaceutically acceptable solvate, ester, enantiomer, diastereomer, N-oxide, polymorph, prodrug or metabolite thereof, wherein
R₁″ and R₂″ are independently selected from C₁-C₆ alkyl, C₃-C₇ cycloalkyl, C₃-C₇ cycloalkenyl or phenyl, wherein phenyl is optionally substituted with one or more groups independently selected from C₁-C₃ alkyl, C₁-C₃ alkoxy or halogen;
R₃′ represents C₁-C₆ alkyl, wherein
1-3 hydrogen atom(s) may be substituted with a group independently selected from C₅-C₇ cycloalkyl, 1,3-dioxo-1,3-dihydro-isoindolyl or phenyl, wherein
phenyl is optionally substituted with one or more groups independently selected C₁-C₄ alkyl or halogen; and
Z represents oxygen or NR₄′, wherein
R₄′ represents hydrogen or C₁-C₃ alkyl.

2. The pharmaceutical composition of claim 1, wherein the one or more MRA are selected from:
(1a,5a,6a)-N-[3-benzyl-3-azabicyclo[3.1.0]hexyl-6-(aminomethyl)-yl]-2-hydroxy-2,2-diphenyl acetamide (Compound No. 1)
(1a,5a,6a)-N-[3-benzyl-3-azabicyclo[3.1.0]hexyl-6-(aminomethyl)-yl]-2-hydroxy-2-cyclohexyl-2-phenyl acetamide (Compound No. 2)
(1a,5a,6a)-N-[3-benzyl-3-azabicyclo[3.1.0]hexyl-6-(aminomethyl)-yl]-2-hydroxy-2-cyclopentyl-2-phenyl acetamide (Compound No. 3)
(1a,5a,6a)-[3-benzyl-3-azabicyclo[3.1.0]hexyl-6-(methyl)-yl]-2-hydroxy-2,2-diphenyl acetate (Compound No. 4)
(1a,5a,6a)-[3-benzyl-3-azabicyclo[3.1.0]hexyl-6-(methyl)-yl]-2-hydroxy-2-cyclohexyl-2-phenyl acetate (Compound No. 5)
(1a,5a,6a)-[3-benzyl-3-azabicyclo[3.1.0]hexyl-6-(methyl)-yl]-2-hydroxy-2-cyclopentyl-2-phenyl acetate (Compound No. 6)
(1a,5a,6a)-[3-(2-(2,3-dihydrobenzofuran-5-yl)ethyl)-3-azabicyclo[3.1.0]hexyl-6-(methyl)-yl]-2-hydroxy-2-cyclohexyl-2-phenyl acetate (Compound No. 7)
(1a,5a,6a)-[3-(2-(2,3-dihydrobenzofuran-5-yl)ethyl)-3-azabicyclo[3.1.0]hexyl-6-(methyl)-yl]-2-hydroxy-2-cyclopentyl-2-phenyl acetate (Compound No. 8)
(1a,5a,6a)-N-[3-(2-(2,3-dihydrobenzofuran-5-yl)ethyl)-3-azabicyclo[3.1.0]hexyl-6-(aminomethyl)-yl]-2-hydroxy-2-cyclohexyl-2-phenyl acetamide (Compound No. 9)
(1a,5a,6a)-N-[3-(2-(2,3-dihydrobenzofuran-5-yl)ethyl)-3-azabicyclo[3.1.0]hexyl-6-(aminomethyl)-yl]-2-hydroxy-2-cyclopentyl-2-phenyl acetamide (Compound No. 10)
(1a,5a,6a)-[3-(2-(3,4-methylenedioxyphenyl)ethyl)-3-azabicyclo[3.1.0]hexyl-6-(methyl)-yl]-2-hydroxy-2-cyclopentyl-2-phenyl acetate (Compound No. 11)
(1a,5a,6a)-[3-(2-(3,4-methylenedioxyphenyl)ethyl)-3-azabicyclo[3.1.0]hexyl-6-(methyl)-yl]-2-hydroxy-2-cyclohexyl-2-phenyl acetate (Compound No. 12)
(1a,5a,6a)-N-[3-(2-(3,4-methylenedioxyphenyl)ethyl)-3-azabicyclo[3.1.0]hexyl-6-(aminomethyl)-yl]-2-hydroxy-2-cyclopentyl-2-phenyl acetamide (Compound No. 13)
(1a,5a,6a)-N-[3-(2-(3,4-methylenedioxyphenyl)ethyl)-3-azabicyclo[3.1.0]hexyl-6-(aminomethyl)-yl]-2-hydroxy-2-cyclohexyl-2-phenyl acetamide (Compound No. 14),
(1a,5a,6a)-N-[3-(4-methyl-3-pentenyl)-3-azabicyclo[3.1.0]hexyl-6-(aminomethyl)-yl]-2-hydroxy-2-cyclohexyl-2-phenyl acetamide (Compound No. 15)
(1a,5a,6a)-N-[3-(4-methyl-3-pentenyl)-3-azabicyclo[3.1.0]hexyl-6-(aminomethyl)-yl]-2-hydroxy-2-cyclopentyl-2-phenyl acetamide (Compound No. 16)
(1a,5a,6a)-[3-(4-methyl-3-pentenyl)-3-azabicyclo[3.1.0]hexyl-6-(methyl)-yl]-2-hydroxy-2-cyclohexyl-2-phenyl acetate (Compound No. 17)
(1a,5a,6a)-[3-(4-methyl-3-pentenyl)-3-azabicyclo[3.1.0]hexyl-6-(methyl)-yl]-2-hydroxy-2-cyclopentyl-2-phenyl acetate (Compound No. 18)
(1a,5a,6a)-[3-(1-phenylethyl)-3-azabicyclo[3.1.0]hexyl-6-(methyl)-yl]-2-hydroxy-2-cyclopentyl-2-phenyl acetate (Compound No. 19)
(1a,5a,6a)-[3-(1-phenylethyl)-3-azabicyclo[3.1.0]hexyl-6-(methyl)-yl]-2-hydroxy-2-cyclohexyl-2-phenyl acetate (Compound No. 20)
(1a,5a,6a)-N-[3-(1-phenylethyl)-3-azabicyclo[3.1.0]hexyl-6-(aminomethyl)-yl]-2-hydroxy-2-cyclohexyl-2-phenyl acetamide (Compound No. 21)
(1a,5a,6a)-N-[3-(1-phenylethyl)-3-azabicyclo[3.1.0]hexyl-6-(aminomethyl)-yl]-2-hydroxy-2-cyclopentyl-2-phenyl acetamide (Compound No. 22)
(1a,5a,6a)-N-[3-benzyl-3-azabicyclo[3.1.0]hexyl-6-(1-aminoethyl)-yl]-2-hydroxy-2,2-diphenyl acetamide (Compound No. 23)
(1a,5a,6a)-N-[3-benzyl-3-azabicyclo[3.1.0]hexyl-6-(1-aminoethyl)-yl]-2-hydroxy-2-cyclohexyl-2-phenyl acetamide (Compound No. 24)
(1a,5a,6a)-N-[3-benzyl-3-azabicyclo[3.1.0]hexyl-6-(1-aminoethyl)-yl]-2-hydroxy-2-cyclopentyl-2-phenyl acetamide (Compound No. 25)
(1a,5a,6a)-[3-(3-methyl-2-butenyl)-3-azabicyclo[3.1.0]hexyl-6-(methyl)-yl]-2-hydroxy-2-cyclohexyl-2-phenyl acetate (Compound No. 26)
(1a,5a,6a)-[3-(3-methyl-2-butenyl)-3-azabicyclo[3.1.0]hexyl-6-(methyl)-yl]-2-hydroxy-2-cyclopentyl-2-phenyl acetate (Compound No. 27)
(2R)-(+)-(1a,5a,6a)-N-[3-benzyl-3-azabicyclo[3.1.0]hexyl-6-(aminomethyl)-yl]-2-hydroxy-2-cyclohexyl-2-phenyl acetamide (Compound No. 28)
(2R)-(+)-(1a,5a,6a)-N-[3-benzyl-3-azabicyclo[3.1.0]hexyl-6-(aminomethyl)-yl]-2-hydroxy-2-cyclopentyl-2-phenyl acetamide (Compound No. 29)
(2R)(+)-(1a,5a,6a)-[3-benzyl-3-azabicyclo[3.1.0]hexyl-6-(methyl)-yl]-2-hydroxy-2-cyclohexyl-2-phenyl acetate (Compound No. 30)
(2R)(+)-(1a,5a,6a)-[3-benzyl-3-azabicyclo[3.1.0]hexyl-6-(methyl)-yl]-2-hydroxy-2-cyclopentyl-2-phenyl acetate (Compound No. 31)
65 (2S)-(−)-(1a,5a,6a)-N-[3-benzyl-3-azabicyclo[3.1.0]hexyl-6-(aminomethyl)-yl]-2-hydroxy-2-cyclopentyl-2-phenyl acetamide (Compound No. 32)
(2S)-(−)-(1a,5a,6a)-[3-benzyl-3-azabicyclo[3.1.0]hexyl-6-(methyl)-yl]-2-hydroxy-2-cyclopentyl-2-phenyl acetate (Compound No. 33)
(1a,5a,6a)-N-[3-benzyl-3-azabicyclo[3.1.0]hexyl-6-(aminomethyl)-yl]-2-hydroxy-2-cyclopentyl-2-phenyl acetamide L-(+)-tartrate salt (Compound No. 34),
(2R)-(+)-(1a,5a,6a)-N-[3-benzyl-3-azabicyclo[3.1.0]hexyl-6-(aminomethyl)-yl]-2-hydroxy-2-cyclohexyl-2-phenyl acetamide. L-(+)-tartrate salt (Compound No. 35)
(2R)-(+)-(1a,5a,6a)-N-[3-benzyl-3-azabicyclo[3.1.0]hexyl-6-(aminomethyl)-yl]-2-hydroxy-2-cyclopentyl-2-phenyl acetamide. L-(+)-tartrate salt (Compound No. 36)
(1a,5a,6a)-N-[3-benzyl-3-azabicyclo[3.1.0]hexyl-6-(aminomethyl)-yl]-2-hydroxy-2-cyclobutyl-2-phenyl acetamide (Compound No. 37)
(1a,5a,6a)-N-[3-benzyl-3-azabicyclo[3.1.0]hexyl-6-(aminomethyl)-yl]-2-hydroxy-2-cyclopropyl-2-phenyl acetamide (Compound No. 38)
(1a,5a,6a)-N-[3-(3-methyl-2-butenyl)-3-azabicyclo[3.1.0]hexyl-6-(aminomethyl)-yl]-2-hydroxy-2-cyclohexyl-2-phenyl acetamide (Compound No. 39)
(1a,5a,6a)-[3-(3,4-methylenedioxyphenyl)methyl-3-azabicyclo[3.1.0]hexyl-6-(methyl)-yl]-2-hydroxy-2-cyclopentyl-2-phenyl acetate (Compound No. 40)
(1a,5a,6a)-[3-(2-(3,4-methylenedioxyphenyl)ethyl)-3-azabicyclo[3.1.0]hexyl-6-(methyl)-yl]-2-hydroxy-2-cyclopentyl-2-phenyl acetate. L-(+)-tartrate salt (Compound No. 41)
(1a,5a,6a)-[3-benzyl-3-azabicyclo[3.1.0]hexyl-6-(methyl)-yl]-2-hydroxy-2,2 diphenyl acetate L(+)-tartrate salt (Compound No. 42),
(1a,5a,6a)-[3-benzyl-3-azabicyclo[3.1.0]hexyl-6-(methyl)-yl]-2-hydroxy-2-cyclohexyl-2-phenyl acetate L(+)-tartrate salt (Compound No. 43)
(1a,5a,6a)-[3-benzyl-3-azabicyclo[3.1.0]hexyl-6-(methyl)-yl]-2-hydroxy-2-cyclopentyl-2-phenyl acetate L(+)-tartrate salt (Compound No. 44)
(1a,5a,6a)-N-[3-(3-pyridylmethyl)-3-azabicyclo[3.1.0]hexyl-6-(aminomethyl)-yl]-2-hydroxy-2-cyclohexyl-2-phenyl acetamide (Compound No. 45)
(1a,5a,6a)-N-[3-(4-pyridylmethyl)-3-azabicyclo[3.1.0]hexyl-6-(aminomethyl)-yl]-2-hydroxy-2-cyclohexyl-2-phenyl acetamide (Compound No. 46)
(1a,5a,6a)-N-[3-(2-pyridylmethyl)-3-azabicyclo[3.1.0]hexyl-6-(aminomethyl)-yl]-2-hydroxy-2-cyclohexyl-2-phenyl acetamide (Compound No. 47)
(1a,5a,6a)-N-[3-(4-pyridylmethyl)-3-azabicyclo[3.1.0]hexyl-6-(aminomethyl)-yl]-2-hydroxy-2-cyclopentyl-2-phenyl acetamide (Compound No. 48)
(1a,5a,6a)-N-[3-(3-pyridylmethyl)-3-azabicyclo[3.1.0]hexyl-6-(aminomethyl)-yl]-2-hydroxy-2,2-diphenyl acetamide (Compound No. 49)
(1a,5a,6a)-N-[3-(4-pyridylmethyl)-3-azabicyclo[3.1.0]hexyl-6-(aminomethyl)-yl]-2-hydroxy-2,2-diphenyl acetamide (Compound No. 50)
(1a,5a,6a)-N-[3-(2-pyridylmethyl)-3-azabicyclo[3.1.0]hexyl-6-(aminomethyl)-yl]-2-hydroxy-2,2-diphenyl acetamide (Compound No. 51)
(1a,5a,6a)-N-[3-(2-pyridylmethyl)-3-azabicyclo[3.1.0]hexyl-6-(aminomethyl)-yl]-2-hydroxy-2-cyclopentyl-2-phenyl acetamide (Compound No. 52)
(1a,5a,6a)-N-[3-(3-pyridylmethyl)-3-azabicyclo[3.1.0]hexyl-6-(aminomethyl)-yl]-2-hydroxy-2-cyclopentyl-2-phenyl acetamide (Compound No. 53)
(1a,5a,6a)-N-[3-(3-methyl-2-butenyl)-3-azabicyclo[3.1.0]hexyl-6-(aminomethyl)-yl]-2-hydroxy-2-cyclopentyl-2-phenyl acetamide (Compound No. 54),
(1a,5a,6a)-N-[3-(3,4-methylenedioxyphenyl)methyl-3-azabicyclo[3.1.0]hexyl-6-(aminomethyl)-yl]-2-hydroxy-2-cyclopentyl-2-phenyl acetamide (Compound No. 55)
(1a,5a,6a)-N-[3-(3,4-methylenedioxyphenyl)methyl-3-azabicyclo[3.1.0]hexyl-6-(aminomethyl)-yl]-2-hydroxy-2-cyclohexyl-2-phenyl acetamide (Compound No. 56)
(1a,5a,6a)-[3-(4-methyl-3-pentenyl)-3-azabicyclo[3.1.0]hexyl-6-(methyl)-yl]-2-hydroxy-2-cyclohexyl-2-phenyl acetate. L(+) tartrate salt (Compound No. 57)
(1a,5a,6a)-[3-(2-(3,4-methylenedioxyphenyl)ethyl)-3-azabicyclo[3.1.0]hexyl-6-(methyl)-yl]-2-hydroxy-2-cyclohexyl-2-phenyl acetate. L(+) tartrate salt (Compound No. 58)
(1a,5a,6a)-[3-(1-phenylethyl)-3-azabicyclo[3.1.0]hexyl-6-(methyl)-yl]-2-hydroxy-2-cyclopentyl-2-phenyl acetate. L(+) tartrate salt (Compound No. 59)
(1a,5a,6a)-N-[3-benzyl-3-azabicyclo[3.1.0]-hexyl-6-(aminomethyl)-yl]-2-hydroxy-2-cyclopentyl-2-phenyl acetamide hydrochloride salt (Compound No. 60)
(1a,5a,6a)-N-[3-benzyl-3-azabicyclo[3.1.0]-hexyl-6-(aminomethyl)-yl]-2-hydroxy-2-cyclopentyl-2-phenyl acetamide. L(−) malic acid salt (Compound No. 61)
(1a,5a,6a)-N-[3-benzyl-3-azabicyclo[3.1.0]-hexyl-6-(aminomethyl)-yl]-2-hydroxy-2-cyclopentyl-2-phenyl acetamide. maleate salt (Compound No. 62)
(2R,2S)(1a,5a,6a)-N-[3-azabicyclo[3.1.0]hexyl-6-(aminomethyl)-yl]-2-hydroxy-2-cyclopentyl-2-phenyl acetamide (Compound No. 63)
(2R,2S)(1a,5a,6a)-N-[3-azabicyclo[3.1.0]hexyl-6-(aminomethyl)-yl]-2-hydroxy-2-cyclopentyl-2-phenyl acetamide hydrochloride salt (Compound No. 64),
(2R)-(1a,5a,6a)-N-[3-azabicyclo[3.1.0]hexyl-6-(aminomethyl)-yl]-2-hydroxy-2-cyclopentyl 2-phenyl acetamide (Compound No. 65)
(2R)-(1a,5a,6a)-N-[3-azabicyclo[3.1.0]hexyl-6-(aminomethyl)-yl]-2-hydroxy-2-cyclopentyl 2-phenyl acetamide hydrochloride salt (Compound No. 66)
(2S)-(1a,5a,6a)-N-[3-azabicyclo[3.1.0]hexyl-6-(aminomethyl)-yl]-2-hydroxy-2-cyclopentyl 2-phenyl acetamide (Compound No. 67)
(2S)-(1a,5a,6a)-N-[3-azabicyclo[3.1.0]hexyl-6-(aminomethyl)-yl]-2-hydroxy-2-cyclopentyl 2-phenyl acetamide hydrochloride salt (Compound No. 68)
(2R,2S)(1a,5a,6a)-N-[3-azabicyclo[3.1.0]hexyl-6-(aminomethyl)-yl]-2-methoxy-2-cyclopentyl-2-phenyl acetamide (Compound No. 69)
(2R,2S)(1a,5a,6a)-N-[3-azabicyclo[3.1.0]hexyl-6-(aminomethyl)-yl]-2-hydroxy-2-cycloheptyl-2-phenyl acetamide (Compound No. 70)
(2R,2S)(1a,5a,6a)-N-[3-azabicyclo[3.1.0]hexyl-6-(aminomethyl)-yl]-2-hydroxy-2-cyclobutyl-2-phenyl acetamide (Compound No. 71)
(2R,2S)(1a,5a,6a)-N-[3-azabicyclo[3.1.0]hexyl-6-(aminomethyl)-yl]-2-hydroxy-2-cyclobutyl-2-phenyl acetamide tartarate salt (Compound No. 72)
(2R)(1a,5a,6a)-N-[3-azabicyclo[3.1.0]hexyl-6-(aminomethyl)-yl]-2-hydroxy-2-(3,3-difluorocyclopentyl)-2-phenyl acetamide (Compound No. 73)
(2R,2S)(1a,5a,6a)-N-[3-azabicyclo[3.1.0]hexyl-6-(aminomethyl)-yl]-2-hydroxy-2-(3-fluorocyclopentyl)-2-phenyl acetamide (Compound No. 74)
(2R,2S)(1a,5a,6a)-N-[3-azabicyclo[3.1.0]hexyl-6-(aminomethyl)-yl]-2-hydroxy-2-(3,3-difluorocyclopentyl)-2-phenyl acetamide (Compound No. 75)
(2R,2S)(1a,5a,6a)-N-[3-azabicyclo[3.1.0]hexyl-6-(aminomethyl)-yl]-2-hydroxy-2-(3,3-difluorocyclopentyl)-2-phenyl acetamide tartarate salt (Compound No. 76)
(2R,2S)(1a,5a,6a)-N-[3-azabicyclo[3.1.0]hexyl-6-(aminomethyl)-yl]-2-hydroxy-2,2-diphenyl acetate (Compound No. 77)
(2R,2S)(1a,5a,6a)-N-[3-azabicyclo[3.1.0]hexyl-6-(aminomethyl)-yl]-2-hydroxy-2,2-diphenyl acetamide (Compound No. 78)
(2R,2S)(1a,5a,6a)-N-[3-azabicyclo[3.1.0]hexyl-6-(aminomethyl)-yl]-2-hydroxy-2-cyclohexyl-2-phenyl acetamide (Compound No. 79)
(2R,2S)(1a,5a,6a)-N-[3-azabicyclo[3.1.0]hex-6-ylmethyl)-2-cyclopentyl-2-hydroxy-N-methyl-2-phenyl acetamide (Compound No. 80)
N-[(1a,5a,6a)-3-azabicyclo[3.1.0]hex-6-yl-methyl]-2-phenyl-2-hydroxy-2-(N-methyl) phenylacetamide (Compound No. 81)
N-[(1α,5α,6α)-3-azabicyclo[3.1.0]hex-6-yl-methyl]-2-phenyl-2-hydroxy-2-(N-methyl) phenylacetamide tartarate salt (Compound No. 82)
(2R,2S)—N-[(1α,5α,6α)-3-azabicyclo[3.1.0]hex-6-yl-methyl]-2-isopropyl-2-hydroxy-2-phenylacetamide (Compound No. 83)
(2R,2S)—N-[(1α,5α,6α)-3-azabicyclo[3.1.0]hex-6-yl-methyl]-2-isopropyl-2-hydroxy-2-phenylacetamide hydrochloride salt (Compound No. 84)
(2R,2S)—N-[(1α,5α,6α)-3-azabicyclo[3.1.0]hex-6-yl-methyl]-2-(3-pentyl)-2-hydroxy-2-phenyl acetamide (Compound No. 85)
(2R,2S)-[(1α,5α,6α)-3-azabicyclo[3.1.0]hex-6-yl-methyl]-2-cyclopentyl-2-hydroxy-2-phenyl acetic acid (Compound No. 86)
(2R)—N-[(1α,5α,6α)-3-azabicyclo[3.1.0]hex-6-yl-methyl]-2-cyclopentyl-2-hydroxy-2-(N-methyl)phenylacetamide (Compound No. 87)
(2R)—N-[(1α,5α,6α)-3-azabicyclo[3.1.0]hex-6-yl-methyl]-2-cyclopentyl-2-hydroxy-2-(N-methyl)phenylacetamide hydrochloride salt (Compound No. 88)
(2R,2S)-[(1α,5α,6α)-3-azabicyclo[3.1.0]hex-6-yl-methyl]-2-methyl-2-hydroxy-2-phenylacetic acid ester (Compound No. 89)
(2R,2S)-[(1α,5α,6α)-3-azabicyclo[3.1.0]hex-6-yl-methyl]-2-isopropyl-2-hydroxy-2-phenylacetic acid ester (Compound No. 90)
(2R,2S)-[(1a, 50a,6a)-3-azabicyclo[3.1.0]hex-6-yl-methyl]-2-(3-pentyl)-2-hydroxy-2-phenylacetic acid ester (Compound No. 91)
(2R,2S)—N-[(1α,5α,6α)-3-azabicyclo[3.1.0]hex-6-yl-methyl]-2-methyl-2-hydroxy-2-phenylacetamide (Compound No. 92)
(2R)—N-[(1α,5α,6α)-3-azabicyclo[3.1.0]hex-6-yl-methyl]-2-isopropyl-2-hydroxy-2-(N-methyl)phenylacetamide (Compound No. 93)
(2R,2S)-[(1α,5α,6α)-3-azabicyclo[3.1.0]hex-6-yl-methyl]-2-(m-methylphenyl)-2-hydroxy-2-phenylacetic acid ester (Compound No. 94)
(2R,2S)—N-[1α,5α,6α)-3-azabicyclo[3.1.0]hex-6-yl-methyl]-2-(p-fluorophenyl)-2-hydroxy-2-phenylacetamide (Compound No. 95)
(2R,2S)—N-[(1α,5α,6α)-3-azabicyclo[3.1.0]hex-6-yl-methyl]-2-(p-methylphenyl)-2-hydroxy-2-phenylacetamide (Compound No. 96)
(2R)—N-[(1α,5α,6α)-3-azabicyclo[3.1.0]hex-6-yl-methyl]-2-(p-fluorophenyl)-2-hydroxy-2-(N-methyl)phenylacetamide (Compound No. 97)
(2R)—N-[(1α,5α,6α)-3-azabicyclo[3.1.0]hex-6-yl-methyl]-2-(p-methylphenyl)-2-hydroxy-2-(N-methyl)phenylacetamide (Compound No. 98)
(2R,2S)(1a,5a,6a)-N-{-[4-(1,3-dioxo-1,3-dihydro-isoindol-2-yl)-butyl]-3-azabicyclo [3.1.0]hex-6-yl-methyl}-2-hydroxy-2-cyclopentyl-2-phenylacetamide (Compound No. 99)
(2R)(1a,5a,6a)-N-(3-Benzyl-3-azabicyclo[3.1.0]hex-6-yl-methyl)-2-hydroxy-2-cyclopent-1-enyl-2-phenylacetamide (Compound No. 100)
(2R,2S)(1a,5a,6a)-N-(3-Isopropyl-3-azabicyclo[3.1.0]hex-6-yl-methyl)-2-hydroxy-2-cyclopentyl-2-phenylacetamide (Compound No. 101)
(2R,2S)(1a,5a,6a)-N-(3-Diphenylmethyl-3-azabicyclo[3.1.0]hex-6-yl-methyl)-2-hydroxy-2-cyclopentyl-2-phenylacetamide (Compound No. 102)
(2R,2S)(1a,5a,6a)-N-(3-sec-butyl-3-azabicyclo[3.1.0]hex-6-yl-methyl)-2-hydroxy-2-cyclopentyl-2-phenylacetamide (Compound No. 103)
(2R,2S)(1a,5a,6a)-N-(3-Benzyl-3-azabicyclo[3.1.0]hex-6-yl-methyl)-2-hydroxy-2-(3-pentyl)-2-phenylacetamide (Compound No. 104)
(2R,2S)(1a,5a,6a)-N-(3-Benzyl-3-azabicyclo[3.1.0]hex-6-yl-methyl)-2-hydroxy-2-cyclohexyl-2-(4-methoxyphenyl)acetamide (Compound No. 105)
(1a,5a,6a)-N-(3-Benzyl-3-azabicyclo[3.1.0]hex-6-yl-methyl)-2-hydroxy-2-phenyl-(N-ethyl)-2-phenylacetamide (Compound No. 106)
(2R,2S)(1a,5a,6a)-N-(3-Benzyl-3-azabicyclo[3.1.0]hex-6-yl-methyl)-2-hydroxy-2-cyclopentyl-(N-ethyl)-2-phenylacetamide (Compound No. 107)
(2R,2S)(1a,5a,6a)-N-(3-Benzyl-3-azabicyclo[3.1.0]hex-6-yl-methyl)-2-hydroxy-2-cyclohexyl-(N-ethyl)-2-phenylacetamide (Compound No. 108)
(2R,2S)(1a,5a,6a)-N-(3-Benzyl-3-azabicyclo[3.1.0]hex-6-yl-methyl)-2-hydroxy-2-(3-pentyl)-(N-methyl)-2-phenylacetamide (Compound No. 109)
(2R,2S)(1a,5a,6a)-N-(3-Benzyl-3-azabicyclo[3.1.0]hex-6-yl-methyl)-2-hydroxy-2-(sec-butyl)-(N-methyl)-2-phenylacetamide (Compound No. 110)
(2R,2S)(1a,5a,6a)-N-(3-Benzyl-3-azabicyclo[3.1.0]hex-6-yl-methyl)-2-hydroxy-2-isopropyl-(N-methyl)-2-phenylacetamide (Compound No. 111)
(2R,2S)(1a,5a,6a)-N-[3-(4-tert-butyl-benzyl)-3-azabicyclo[3.1.0]hex-6-yl-methyl]-2-hydroxy-2-cyclopentyl-2-phenylacetamide (Compound No. 112)
(2R,2S)(1a,5a,6a)-N-(3-Benzyl-3-azabicyclo[3.1.0]hex-6-yl-methyl)-2-hydroxy-2-cyclohex-2-enyl-2-phenylacetamide (Compound No. 113)
(1a,5a,6a)-N-[3-(4-methylbenzyl)-3-azabicyclo[3.1.0]hex-6-yl-methyl]-2-hydroxy-2,2-diphenylacetamide (Compound No. 114)
(2R,2S)(1a,5a,6a)-N-[3-(4-methylbenzyl)-3-azabicyclo[3.1.0]hex-6-yl-methyl]-2-hydroxy-2-cyclopentyl-2-phenylacetamide (Compound No. 115)
(2R,2S)(1a,5a,6a)-N-[3-(4-methylbenzyl)-3-azabicyclo[3.1.0]hex-6-yl-methyl]-2-hydroxy-2-cyclohexyl-2-phenylacetamide (Compound No. 116)
(1a,5a,6a)-N-[3-(3-methylbenzyl)-3-azabicyclo[3.1.0]hex-6-yl-methyl]-2-hydroxy-2,2-diphenylacetamide (Compound No. 117)
(1a,5a,6a)-N-[3-(3-fluorobenzyl)-3-azabicyclo[3.1.0]hex-6-yl-methyl]-2-hydroxy-2,2-diphenylacetamide (Compound No. 118)
(2R,2S)(1a,5a,6a)-N-[3-(3-fluorobenzyl)-3-azabicyclo[3.1.0]hex-6-yl-methyl]-2-hydroxy-2-cyclohexyl-2-phenylacetamide (Compound No. 119)
(2R,2S)(1a,5a,6a)-N-[2-(2,4-difluorobenzyl)-3-azabicyclo[3.1.0]hex-6-yl-methyl]-2-hydroxy-2-cyclohexyl-2-phenylacetamide (Compound No. 120)
(1a,5a,6a)-N-[3-(2,4-difluorobenzyl)-3-azabicyclo[3.1.0]hex-6-yl-methyl]-2-hydroxy-2,2-diphenylacetamide (Compound No. 121)
(2R,2S)(1a, 5a,6a)-N-[3-(3-methylbenzyl)-3-azabicyclo[3.1.0]hex-6-yl-methyl]-2-hydroxy-2-cyclopentyl-2-phenylacetamide (Compound No. 122)
(2R,2S)(1a,5a,6a)-N-(3-benzyl-3-azabicyclo[3.1.0]hex-6-yl-methyl)-2-hydroxy-2-(4-methylphenyl)-2-phenylacetamide (Compound No. 123)
(2R,2S)(1a,5a,6a)-N-(3-benzyl-3-azabicyclo[3.1.0]hex-6-yl-methyl)-2-hydroxy-2-(4-methylphenyl)-(N-methyl)-2-phenylacetamide (Compound No. 124)
(2R,2S)(1a,5a,6a)-N-(3-benzyl-3-azabicyclo[3.1.0]hex-6-yl-methyl)-2-hydroxy-2-(4-fluorophenyl)-2-phenylacetamide (Compound No. 125)
(2R,2S)(1a,5a,6a)-(3-benzyl-3-azabicyclo[3.1.0]hex-6-yl-methyl)-2-hydroxy-2-(4-fluorophenyl)-2-phenyl acetic acid ester (Compound No. 126)
(2R,2S)(1a,5a,6a)-N-(3-benzyl-3-azabicyclo[3.1.0]hex-6-yl-methyl)-2-hydroxy-2-(4-fluorophenyl)-(N-methyl)-2-phenylacetamide (Compound No. 127)
(2R,2S)(1a,5a,6a)-N-(3-benzyl-3-azabicyclo[3.1.0]hex-6-yl-methyl)-2-hydroxy-2-(3-methylphenyl)-2-phenylacetamide (Compound No. 128)
(2R,2S)(1a,5a,6a)-N-(3-benzyl-3-azabicyclo[3.1.0]hex-6-yl-methyl)-2-hydroxy-2-(3-methylphenyl)-(N-methyl)-2-phenylacetamide (Compound No. 129)
(2R,2S)(1a,5a,6a)-(3-benzyl-3-azabicyclo[3.1.0]hex-6-yl-methyl)-2-hydroxy-2-(3-methylphenyl)-2-phenyl acetic acid ester (Compound No. 130)
(2R,2S)(1a,5a,6a)-(3-benzyl-3-azabicyclo[3.1.0]hex-6-yl-methyl)-2-hydroxy-2-cyclopentyl-2-(3-methylphenyl)acetic acid ester (Compound No. 131)
(2R,2S)(1a,5a,6a)-(3-benzyl-3-azabicyclo[3.1.0]hex-6-yl-methyl)-2-hydroxy-2-cyclopentyl-2-(3-methylphenyl)acetic acid ester tartarate salt (Compound No. 132)
(2R,2S)(1a,5a,6a)-N-(3-benzyl-3-azabicyclo[3.1.0]hex-6-yl-methyl)-2-hydroxy-2-cyclopentyl-2-(3-methylphenyl)acetamide (Compound No. 133)
(2R,2S)(1a,5a,6a)-N-(3-benzyl-3-azabicyclo[3.1.0]hex-6-yl-methyl)-2-hydroxy-2-cyclopentyl-2-(3-methylphenyl)acetamide tartarate salt (Compound No. 134),
(1a,5a,6a)-N-(3-benzyl-3-azabicyclo[3.1.0]hex-6-yl-methyl)-2-hydroxy-2,2-di(4-fluorophenyl)acetic acid ester (Compound No. 135)
(1a,5a,6a)-N-(3-benzyl-3-azabicyclo[3.1.0]hex-6-yl-methyl]-2-hydroxy-2,2-di(4-fluorophenyl)-acetamide (Compound No. 136)
(2R,2S)(1a,5a,6a)-(3-benzyl-3-azabicyclo[3.1.0]hex-6-yl-methyl]-2-hydroxy-2-cyclobutyl-2-phenyl acetic acid ester (Compound No. 137)
(2R,2S)(1a,5a,6a)-N-(3-cyclohexylmethyl-3-azabicyclo[3.1.0]hex-6-yl-methyl)-2-hydroxy-2-cyclopentyl-2-phenylacetamide (Compound No. 138)
(2R)(1a,5a,6a)-N-(3-benzyl-3-azabicyclo[3.1.0]hex-6-yl-methyl)-2-hydroxy-2-cyclopentyl-(N-methyl)-2-phenylacetamide (Compound No. 139)
(2R,2S)(1a,5a,6a)-N-(3-benzyl-3-azabicyclo[3.1.0]hex-6-yl-methyl]-2-hydroxy-2-cyclopentyl-2-(4-methylphenyl)acetamide (Compound No. 140)
(2R,2S)(1a,5a,6a)-(3-benzyl-3-azabicyclo[3.1.0]hex-6-yl-methyl)-2-hydroxy-2-phenyl-2-(4-methylphenyl)acetic acid ester (Compound No. 141)
(2R,2S)(1a,5a,6a)-(3-benzyl-3-azabicyclo[3.1.0]hex-6-yl-methyl)-2-hydroxy-2-methyl-2-phenyl acetic acid ester (Compound No. 142)
(2R,2S)(1a,5a,6a)-N-(3-benzyl-3-azabicyclo[3.1.0]hex-6-yl-methyl]-2-hydroxy-2-methyl-2-phenyl acetamide (Compound No. 143)
(2R,2S)(1a,5a,6a)-(3-benzyl-3-azabicyclo[3.1.0]hex-6-yl-methyl)-2-hydroxy-2-isopropyl-2-phenyl acetic acid ester (Compound No. 144),
(1a,5a,6a)-N-(3-methyl-3-azabicyclo[3.1.0]hex-6-yl-methyl]-2-hydroxy-2-phenyl-(N-methyl)-2-phenylacetamide (Compound No. 145),
(1a,5a,6a)-N-(3-benzyl-3-azabicyclo[3.1.0]hex-6-yl-methyl]-2-hydroxy-2,2-di (3-methylphenyl)acetamide (Compound No. 146),
(2R,2S)(1a,5a,6a)-(3-benzyl-3-azabicyclo[3.1.0]hex-6-yl-methyl]-2-hydroxy-2-(3-pentyl)-2-phenyl acetic acid ester (Compound No. 147),
(2R,2S)(1a,5a,6a)-N-(3-benzyl-3-azabicyclo[3.1.0]hex-6-yl-methyl)-2-hydroxy-2-methyl-(N-methyl)-2-phenylacetamide (Compound No. 148),
N-[(1α,5α,6α)-3-azabicyclo[3.1.0]hex-6-yl-methyl]-2-phenyl-2-hydroxy-2-(N-methyl)phenyl acetamide hydrochloride (Compound No. 149), or
Tartarate salt of (3-benzyl-3-azabicyclo[3.1.0]hex-6-yl)methyl cyclopentyl(hydroxy)2-thienylacetate (Compound No. 150).

3. The pharmaceutical composition of claim 1, wherein the one or more PDE-IV inhibitors are selected from:
3-[3-{[(3S)-1-Benzylpyrrolidin-3-yl]oxy}-4-(difluoromethoxy)phenyl]-1,7-dioxa-2-azaspiro[4.4]non-2-ene (Compound No. 1a),
3-[2-(Difluoromethoxy)-5-(1,7-dioxa-2-azaspiro[4.4]non-2-en-3-yl)phenoxy]propan-1-ol (Compound No. 2a),
[2-(Difluoromethoxy)-5-(1,7-dioxa-2-azaspiro[4.4]non-2-en-3-yl)phenoxy]acetonitrile (Compound No. 3a),
4-[(5S or 5R)-1,7-dioxa-2-azaspiro[4.4]non-2-en-3-yl]-2-methoxyphenol (Compound No. 4a),
4-[(5R or 5S)-1,7-Dioxa-2-azaspiro[4.4]non-2-en-3-yl]-2-methoxyphenol (Compound No. 5a),
5-[(5S or 5R)-1,7-Dioxa-2-azaspiro[4.4]non-2-en-3-yl]-2-methoxyphenol (Compound No. 6a),
(5S or 5R)-3-(3,4-Dimethoxyphenyl)-1,7-dioxa-2-azaspiro[4.4]non-2-ene (Compound No. 7a),
(5R or 5S)-3-(3,4-Dimethoxyphenyl)-1,7-dioxa-2-azaspiro[4.4]non-2-ene (Compound No. 8a),
2-(Benzyloxy)-4-(1,7-dioxa-2-azaspiro[4.4]non-2-en-3-yl)phenol (Compound No. 9a),
2-[2-(Difluoromethoxy)-5-(1,7-dioxa-2-azaspiro[4.4]non-2-en-3-yl)phenoxy]ethanol (Compound No. 10a),
3-[4-(Difluoromethoxy)-3-ethoxyphenyl]-1,7-dioxa-2-azaspiro[4.4]non-2-ene (Compound No.
3-[3-(Cyclohexyloxy)-4-(difluoromethoxy)phenyl]-1,7-dioxa-2-azaspiro[4.4]non-2-ene (Compound No. 12a),
(5R or 5S)-3-[4-(Difluoromethoxy)-3-methoxyphenyl]-1,7-dioxa-2-azaspiro[4.4]non-2-ene (Compound No. 13a),
(5S or 5R)-3-[4-(Difluoromethoxy)-3-methoxyphenyl]-1,7-dioxa-2-azaspiro[4.4]non-2-ene (Compound No. 14a),
Ethyl [2-(difluoromethoxy)-5-(1,7-dioxa-2-azaspiro[4.4]non-2-en-3-yl)phenoxy]acetate (Compound No. 15a),
3-[4-(Difluoromethoxy)-3-(2-morpholin-4-ylethoxy)phenyl]-1,7-dioxa-2-azaspiro[4.4]non-2-ene (Compound No. 16a),
2-(Difluoromethoxy)-5-(1,7-dioxa-2-azaspiro[4.4]non-2-en-3-ylphenyl cyclohexanecarboxylate (Compound No. 17a),
5-[2-(Difluoromethoxy)-5-(1,7-dioxa-2-azaspiro[4.4]non-2-en-3-yl)phenoxy]pentanoic acid (Compound No. 15a),
3-[3-(2,2,2-Trifluoroethoxy)-4-(difluoromethoxy)phenyl]-1,7-dioxa-2-azaspiro[4.4]non-2-ene (Compound No. 19a),
3-[3-(Cyclopentylmethoxy)-4-(difluoromethoxy)phenyl]-1,7-dioxa-2-azaspiro[4.4]non-2-ene (Compound No. 20a),
N-cyclopropyl-2-[2-(difluoromethoxy)-5-(1,7-dioxa-2-azaspiro[4.4]non-2-en-3-yl)phenoxy]acetamide (Compound No. 21a),
2-[2-(Difluoromethoxy)-5-(1,7-dioxa-2-azaspiro[4.4]non-2-en-3-yl)phenoxy]acetamide (Compound No. 22a),
2-[2-(Difluoromethoxy)-5-(1,7-dioxa-2-azaspiro[4.4]non-2-en-3-yl)phenoxy]-N-methylacetamide (Compound No. 23a),
3-[3-(Cyclopentyloxy)-4-(2,2,2-trifluoroethoxy)phenyl]-1,7-dioxa-2-azaspiro[4.4]non-2-ene (Compound No. 24a).
2-(Difluoromethoxy)-5-(1,7-dioxa-2-azaspiro[4.4]non-2-en-3-yl)phenyl cyclopropanecarboxylate (Compound No. 25a),
2-(Difluoromethoxy)-5-(1,7-dioxa-2-azaspiro[4.4]non-2-en-3-yl)phenyl morpholine-4-carboxylate (Compound No. 26a),
2-(Difluoromethoxy)-5-(1,7-dioxa-2-azaspiro[4.4]non-2-en-3-yl)phenyl benzoate (Compound No. 27a),
5-[2-(Difluoromethoxy)-5-(1,7-dioxa-2-azaspiro[4.4]non-2-en-3-yl)phenoxy] pentanamide (Compound No. 28a),
3-[3-Propoxy-4-(2,2,2-trifluoroethoxy)phenyl]-1,7-dioxa-2-azaspiro[4.4]non-2-ene (Compound No. 29a),
3-[3-Isopropoxy-4-(2,2,2-trifluoroethoxy)phenyl]-1,7-dioxa-2-azaspiro[4.4]non-2-ene (Compound No. 30a),
3-[3-(Cyclopropylmethoxy)-4-(2,2,2-trifluoroethoxy)phenyl]-1,7-dioxa-2-azaspiro[4.4]non-2-ene (Compound No. 31a),
3-[3-(2,3-Dihydro-1H-inden-2-yloxy)-4-(2,2,2-trifluoroethoxy)phenyl]-1,7-dioxa-2-azaspiro[4.4]non-2-ene (Compound No. 32a),
5-(1,7-Dioxa-2-azaspiro[4.4]non-2-en-3-yl)-2-(2,2,2-trifluoroethoxy)phenol (Compound No. 33a),
3-[3-Methoxy-4-(2,2,2-trifluoroethoxy)phenyl]-1,7-dioxa-2-azaspiro[4.4]non-2-ene (Compound No. 34a),
3-[3-Ethoxy-4-(2,2,2-trifluoroethoxy)phenyl]-1,7-dioxa-2-azaspiro[4.4]non-2-ene (Compound No. 35a),
3-[3-Butoxy-4-(2,2,2-trifluoroethoxy)phenyl]-1,7-dioxa-2-azaspiro[4.4]non-2-ene10019955 (Compound No. 36a),
3-[3-(Cyclohexylmethoxy)-4-(2,2,2-trifluoroethoxy)phenyl]-1,7-dioxa-2-azaspiro[4.4]non-2-ene (Compound No. 37a),
3-{[2-(Difluoromethoxy)-5-(1,7-dioxa-2-azaspiro[4.4]non-2-en-3-yl)phenoxy]methyl} benzonitrile (Compound No. 38a),
2-{2-[2-(difluoromethoxy)-5-(1,7-dioxa-2-azaspiro[4.4]non-2-en-3-yl)phenoxy]ethyl}-1H-isoindole-1,3(2H)-dione (Compound No. 39a),
3-[3-(Cyclohexyloxy)-4-(2,2,2-trifluoroethoxy)phenyl]-1,7-dioxa-2-azaspiro[4.4]non-2-ene (Compound No. 40a),
Ethyl [5-(1,7-dioxa-2-azaspiro[4.4]non-2-en-3-yl)-2-(2,2,2-trifluoroethoxy) phenoxy]acetate (Compound No. 41a),
3-[3-(Cyclohexylmethoxy)-4-(difluoromethoxy)phenyl]-1,7-dioxa-2-azaspiro[4.4]non-2-ene (Compound No. 42a),
Tert-butyl [2-(difluoromethoxy)-5-(1,7-dioxa-2-azaspiro[4.4]non-2-en-3-yl)phenoxy]acetate (Compound No. 43a),
N-cyclopropyl-2-[5-(1,7-dioxa-2-azaspiro[4.4]non-2-en-3-yl)-2-(2,2,2-trifluoroethoxy)phenoxy]acetamide (Compound No. 44a),
2-(Cyclopentyloxy)-4-[(5R or 5S)-1,7-dioxa-2-azaspiro[4.4]non-2-en-3-yl]phenol (Compound No. 45a),
2-(Cyclopentyloxy)-4-[(5S or 5R)-1,7-dioxa-2-azaspiro[4.4]non-2-en-3-yl]phenol (Compound No. 46a),
N-benzyl-2-[5-(1,7-dioxa-2-azaspiro[4.4]non-2-en-3-yl)-2-(2,2,2-trifluoroethoxy)phenoxy]acetamide (Compound No. 47a),
N-Cyclopentyl-2-[5-(1,7-dioxa-2-azaspiro[4.4]non-2-en-3-yl)-2-(2,2,2-trifluoroethoxy)phenoxy]acetamide (Compound No. 48a),
tert-Butyl 4-[2-(difluoromethoxy)-5-(1,7-dioxa-2-azaspiro[4.4]non-2-en-3-yl)phenoxy]piperidine-1-carboxylate (Compound No. 49a),
Hydrochloride salt of 3-[4-(difluoromethoxy)-3-(piperidin-4-yloxy)phenyl]-1,7-dioxa-2-azaspiro[4.4]non-2-ene (Compound No. 50a),
3-{3-[(1-Acetylpiperidin-4-yl)oxy]-4-(difluoromethoxy)phenyl}-1,7-dioxa-2-azaspiro[4.4]non-2-ene (Compound No. 51a),
Tert-butyl (3S)-3-[2-(difluoromethoxy)-5-(1,7-dioxa-2-azaspiro[4.4]non-2-en-3-yl)phenoxy]pyrrolidine-1-carboxylate (Compound No. 52a),
Tert-butyl (3R)-3-[2-(difluoromethoxy)-5-(1,7-dioxa-2-azaspiro[4.4]non-2-en-3-yl)phenoxy]pyrrolidine-1-carboxylate (Compound No. 53a),
Tert-butyl 3-[2-(difluoromethoxy)-5-(1,7-dioxa-2-azaspiro[4.4]non-2-en-3-yl)phenoxy]piperidine-1-carboxylate (Compound No. 54a),
Tert-butyl (2S)-2-{[2-(difluoromethoxy)-5-(1,7-dioxa-2-azaspiro[4.4]non-2-en-3-yl)phenoxy]methyl}pyrrolidine-1-carboxylate (Compound No. 55a),
(5R or 5S)-3-[3-(cyclopentyloxy)-4-(difluoromethoxy)phenyl]-1,7-dioxa-2-azaspiro[4.4]non-2-ene (Compound No. 56a),
(5S or 5R)-3-(3-isopropoxy-4-methoxyphenyl)-1,7-dioxa-2-azaspiro[4.4]non-2-ene (Compound No. 57a),
(5S or 5R)-3-[3-(Cyclopropylmethoxy)-4-methoxyphenyl]-1,7-dioxa-2-azaspiro[4.4]non-2-ene (Compound No. 58a),
2-(Cyclopropylmethoxy)-4-[(5S or 5R)-1,7-dioxa-2-azaspiro[4.4]non-2-en-3-yl]phenol (Compound No. 59a),
4-[(5S or 5R)-1,7-Dioxa-2-azaspiro[4.4]non-2-en-3-yl]-2-isopropoxyphenol (Compound No. 60a),
(5S or 5R)-3-[3-(cyclopentyloxy)-4-(difluoromethoxy)phenyl]-1,7-dioxa-2-azaspiro[4.4]non-2-ene (Compound No. 61a),
(5S or 5R)-3-[3-(Cyclopropylmethoxy)-4-(difluoromethoxy)phenyl]-1,7-dioxa-2-azaspiro[4.4]non-2-ene (Compound No. 62a),
(5S or 5R)-3-[4-(difluoromethoxy)-3-isopropoxyphenyl]-1,7-dioxa-2-azaspiro[4.4]non-2-ene (Compound No. 63a),
(5R or 5S)-3-[4-(difluoromethoxy)-3-isopropoxyphenyl]-1,7-dioxa-2-azaspiro[4.4]non-2-ene (Compound No. 64a),
2-(Cyclopropylmethoxy)-4-[(5R or 5S)-1,7-dioxa-2-azaspiro[4.4]non-2-en-3-yl]phenol (Compound No. 65a),
4-[(5R or 5S)-1,7-Dioxa-2-azaspiro[4.4]non-2-en-3-yl]-2-isopropoxyphenol (Compound No. 66a),
(5R or 5S)-3-[3-(Cyclopropylmethoxy)-4-(difluoromethoxy)phenyl]-1,7-dioxa-2-azaspiro[4.4]non-2-ene (Compound No. 67a),
(5R or 5S)-3-[4-(difluoromethoxy)-3-isopropoxyphenyl]-1,7-dioxa-2-azaspiro[4.4]non-2-ene (Compound No. 68a),
Hydrochloride salt of 3-{4-(difluoromethoxy)-3-[(3S)-pyrrolidin-3-yloxy]phenyl}-1,7-dioxa-2-azaspiro[4.4]non-2-ene (Compound No. 69a),
Hydrochloride salt of 3-{4-(difluoromethoxy)-3-[(2S)-pyrrolidin-2-ylmethoxy]phenyl}-1,7-dioxa-2-azaspiro[4.4]non-2-ene (Compound No. 70a),
Hydrochloride salt of 3-{4-(difluoromethoxy)-3-[(2R)-pyrrolidin-2-ylmethoxy]phenyl}-1,7-dioxa-2-azaspiro[4.4]non-2-ene (Compound No. 71a),
3-[4-(Difluoromethoxy)-3-{[(2R)-1-propionylpyrrolidin-2-yl]methoxy}phenyl]-1,7-dioxa-2-azaspiro[4.4]non-2-ene (Compound No. 72a),
3-[3-{[(2S)-1-acetylpyrrolidin-2-yl]methoxy}-4-(difluoromethoxy)phenyl]-1,7-dioxa-2-azaspiro[4.4]non-2-ene (Compound No. 73a),
3-{[3-[(3S )-1-benzoylpyrrolidin-3-yl]oxy}-4-(difluoromethoxy)phenyl]-1,7-dioxa-2-azaspiro[4.4]non-2-ene (Compound No. 74a),
3-[4-(Difluoromethoxy)-3-{[(3S)-1-propionylpyrrolidin-3-yl]oxy}phenyl]-1,7-dioxa-2-azaspiro[4.4]non-2-ene (Compound No. 75a),
(5S or 5R)-3-[3-(Benzyloxy)-4-(difluoromethoxy)phenyl]-1,7-dioxa-2-azaspiro[4.4]non-2-ene (Compound No. 76a),
2-(Benzyloxy)-4-[(5S or 5R)-1,7-dioxa-2-azaspiro[4.4]non-2-en-3-yl]phenol (Compound No. 77a),
(5S or 5R)-3-[3-(Benzyloxy)-4-methoxyphenyl]-1,7-dioxa-2-azaspiro[4.4]non-2-ene (Compound No. 78a),
3-{4-(Difluoromethoxy)-3-[(1-propionylpiperidin-4-yl)oxy]phenyl}-1,7-dioxa-2-azaspiro[4.4]non-2-ene (Compound No. 79a),
3-[4-(Difluoromethoxy)-3-{[1-(4-fluorobenzoyl)piperidin-4-yl]oxy}phenyl]-1,7-dioxa-2-azaspiro[4.4]non-2-ene (Compound No. 80a),
3-[3-{[1-(Cyclopropylcarbonyl)piperidin-4-yl]oxy}-4-(difluoromethoxy)phenyl]-1,7-dioxa-2-azaspiro[4.4]non-2-ene (Compound No. 81a),
3-[3-{[1-(Cyclopentylcarbonyl)piperidin-4-yl]oxy}-4-(difluoromethoxy)phenyl]-1,7-dioxa-2-azaspiro[4.4]non-2-ene (Compound No. 82a),
3-[4-(Difluoromethoxy)-3-({1-[(trifluoromethyl)sulfonyl]piperidin-4-yl}oxy)phenyl]-1,7-dioxa-2-azaspiro[4.4]non-2-ene (Compound No. 83a),
3-{3-[(1-Acetylpiperidin-3-yl)oxy]-4-(difluoromethoxy)phenyl}-1,7-dioxa-2-azaspiro[4.4]non-2-ene (Compound No. 84a),
3-{4-(Difluoromethoxy)-3-[(1-propionylpiperidin-3-yl)oxy]phenyl}-1,7-dioxa-2-azaspiro[4.4]non-2-ene (Compound No. 85a),
3-[4-(Difluoromethoxy)-3-{[1-(4-fluorobenzoyl)piperidin-3-yl]oxy}phenyl]-1,7-dioxa-2-azaspiro[4.4]non-2-ene (Compound No. 86a),
3-[3-{[1-(Cyclopropylcarbonyl)piperidin-3-yl]oxy}-4-(difluoromethoxy)phenyl]-1,7-dioxa-2-azaspiro[4.4]non-2-ene (Compound No. 87a),
3-[3-{[1-(Cyclopentylcarbonyl)piperidin-3-yl]oxy}-4-(difluoromethoxy)phenyl]-1,7-dioxa-2-azaspiro[4.4]non-2-ene (Compound No. 88a),
3-[4-(Difluoromethoxy)-3-{[1-(ethylsulfonyl)piperidin-3-yl]oxy}phenyl]-1,7-dioxa-2-azaspiro[4.4]non-2-ene (Compound No. 89a),
3-[3-(Benzyloxy)-4-(2,2,2-trifluoroethoxy)phenyl]-1,7-dioxa-2-azaspiro[4.4]non-2-ene (Compound No. 90a),
2-(Difluoromethoxy)-5-[(5S or 5R)-1,7-dioxa-2-azaspiro[4.4]non-2-en-3-yl]phenol (Compound No. 91a),
5-[5R or 5S)-1,7-Dioxa-2-azaspiro[4.4]non-2-en-3-yl]-2-methoxyphenol (Compound No. 92a),
3-[3-(cyclopentyloxy)-4-methoxyphenyl]-1,7-dioxa-2-azaspiro[4.4]non-2-en-6-ol (Compound No. 1aa),
3-[3-(Cyclopentyloxy)-4-methoxyphenyl]-N-(4-fluorophenyl)-1-oxa-2,7-diazaspiro[4.4]non-2-ene-7-carboxamide (Compound No. 2aa),
3-[3-(Cyclopentyloxy)-4-methoxyphenyl]-7-(tetrahydrofuran-3-ylcarbonyl)-1-oxa-2,7-diazaspiro[4.4]non-2-ene (Compound No. 3aa),
3-[3-(cyclopentyloxy)-4-methoxyphenyl]-N,N-dimethyl-1-oxa-2,7-diazaspiro[4.4]non-2-ene-7-sulfonamide (Compound No. 4aa),
N-butyl-3-[3-(cyclopentyloxy)-4-methoxyphenyl]-1-oxa-2,7-diazaspiro[4.4]non-2-ene-7-carboxamide (Compound No. 5aa),
2-{3-[3-(Cyclopentyloxy)-4-methoxyphenyl]-1-oxa-2,7-diazaspiro[4.4]non-2-en-7-yl}acetamide (Compound No. 6aa),
Hydrochloride salt of 3-[3-(cyclopentyloxy)-4-methoxyphenyl]-8-prolyl-1-oxa-2,8-diazaspiro[4.5]dec-2-ene (Compound No. 7aa),
3-[3-(Cyclopentyloxy)-4-methoxyphenyl]-8-(2-morpholin-4-yl-ethyl)-1-oxa-2,8-diazaspiro[4.5]dec-2-ene (Compound No. 8aa),
N-butyl-3-[3-(cyclopentyloxy)-4-methoxyphenyl]-1-oxa-2,8-diazaspiro[4.5]dec-2-ene-8-carboxamide (Compound No. 9aa),
3-[3-(cyclopentyloxy)-4-methoxyphenyl]-8-(methylsulfonyl)-1-oxa-2,8-diazaspiro[4.5]dec-2-ene (Compound No. 10aa),
3-[3-(cyclopentyloxy)-4-methoxyphenyl]-1-oxa-2-azaspiro[4.4]non-2-ene (Compound No.
3-[3,4-bis(2-morpholin-4-ylethoxy)phenyl]-1,7-dioxa-2-azaspiro[4.4]non-2-ene (Compound No. 12aa),
3-(3,4-diisopropoxyphenyl)-1,7-dioxa-2-azaspiro[4.4]non-2-ene (Compound No. 13aa),
3-[3-methoxy-4-(2-morpholin-4-ylethoxy)phenyl]-1,7-dioxa-2-azaspiro[4.4]non-2-ene (Compound No. 14aa),
3-[3-(cyclopentyloxy)-4-methoxyphenyl]-1,7-dioxa-2-azaspiro[4.4]non-2-en-8-one (Compound No. 15aa),
3-[3-(cyclopentyloxy)-4-methoxyphenyl]-1,7-dioxa-2-azaspiro[4.4]non-2-en-8-ol (Compound No. 16aa),
3-[3-(Cyclopentyloxy)-4-methoxyphenyl]-7-isopropyl-1-oxa-2,7-diazaspiro[4.4]non-2-ene (Compound No. 17aa),
3-[3-(cyclopentyloxy)-4-methoxyphenyl]-7-(cyclopropylcarbonyl-1-oxa-2,7-diazaspiro[4.4]non-2-ene (Compound No. 18aa),
N-benzyl-3-[3-(cyclopentyloxy)-4-methoxyphenyl]-1-oxa-2,7-diazaspiro[4.4]non-2-ene-7-carboxamide (Compound No. 19aa),
7-acetyl-3-[3-(cyclopentyloxy)-4-methoxyphenyl]-1-oxa-2,7-diazaspiro[4.4]non-2-ene (Compound No. 20aa),
Tert-butyl 3-[3-(cyclopentyloxy)-4-methoxyphenyl]-1-oxa-2,7-diazaspiro[4.5]dec-2-ene-7-carboxylate (Compound No. 21aa),
N-butyl-N′-{3-[3-(cyclopentyloxy-4-methoxyphenyl]-1-oxa-2-azaspiro[4.5]dec-2-en-8-yl}urea (Compound No. 22aa),
N-{3-[3-(cyclopentyloxy)-4-methoxyphenyl]-1-oxa-2-azaspiro[4.5]dec-2-en-5-yl}-N-(2-methoxyphenyl)urea (Compound No. 23aa),
3-[3-(cyclopentyloxy)-4-methoxyphenyl]-1-oxa-2-azaspiro[4.5]dec-2-en-8-ol (Compound No. 24
Hydrochloride salt of 3-[3-(cyclopentyloxy)-4-methoxyphenyl]-1-oxa-2,7-diazaspiro[4.5]dec-2-ene (Compound No. 25aa),
3-[3-(cyclopentyloxy)-4-methoxyphenyl]-1-oxa-2-azaspiro[4.5]dec-2-en-8-one (Compound No. 26aa),
3-[3,4-bis(cyclopentyloxy)phenyl]-1,7-dioxa-2-azaspiro[4.4]non-2-ene (Compound No. 27aa),
3-[3,4-Bis(cyclopropylmethoxy)phenyl]-1,7-dioxa-2-azaspiro[4.4]non-2-ene (Compound No. 28aa),
3-[3-(cyclopentyloxy)-4-methoxyphenyl]-1,7-dioxa-2-azaspiro[4.4]non-2-en-4-ol (Compound No. 29aa),
(R)-3-[3-(cyclopentyloxy)-4-methoxyphenyl]-1,7-dioxa-2-azaspiro[4.4]non-2-ene (Compound No. 30aa),
3-[3-(Cyclopentyloxy)-4-methoxyphenyl]-8-(cyclopropylmethyl)-1-oxa-2,8-diazaspiro[4.5]dec-2-ene (Compound No. 31aa),
N-Benzyl-3-[3-(cyclopentyloxy)-4-methoxyphenyl]-1-oxa-2,8-diazaspiro[4.5]dec-2-ene-8-carboxamide (Compound No. 32aa),
3-[3,4-Bis(benzyloxy)phenyl]-1,7-dioxa-2-azaspiro[4.4]non-2-ene (Compound No. 33aa),
4-(1,7-Dioxa-2-azaspiro[4.4]non-2-en-3-yl)benzene-1,2-diol (Compound No. 34aa),
7-Amino-3-[3-(cyclopentyloxy)-4-methoxyphen-yl]-1-oxa-2,7-diazaspiro[4.4]non-2-en-6-one (Compound No. 35aa),
Ethyl 8-benzyl-3-[3-(cyclopentyloxy)-4-methoxyphenyl]-1-oxa-2,8-diazaspiro[4.5]dec-2-ene-4-carboxylate (Compound No. 36aa),
3-[3-(Cyclopentyloxy)-4-methoxyphenyl]-1-oxa-2-azaspiro[4.5]dec-2-ene-4-carboxylic acid (Compound no. 37aa),
8-Benzyl-3-[3-(cyclopentyloxy)-4-methoxyphenyl]-1-oxa-2,8-diazaspiro[4.5]dec-2-ene (Compound No. 38aa),
Ethyl 3-[3-(cyclopentyloxy)-4-methoxyphenyl]-1-oxa-2-azaspiro[4.5]dec-2-ene-4-carboxylate (Compound No. 39aa),
3-[3-(Difluoromethoxy)-4-methoxyphenyl]-1,7-dioxa-2-azaspiro[4.4]non-2-ene (Compound No. 40aa),
2-(Difluoromethoxy)-5-(1,7-dioxa-2-azaspiro[4.4]non-2-en-3-yl)phenol (Compound No. 41aa),
3-[3-(Cyclopentyloxy)-4-methoxyphenyl]-1-oxa-2,7-diazaspiro[4.4]non-2-en-6-one (Compound No. 42aa),
3-[3-(Cyclopentyloxy)-4-methoxyphenyl]-3a,6a-dimethyl-3aH-cyclopenta[d]isoxazole-4,6(5H,6aH-dione (Compound No. 43aa),
3-[3-(Cyclopentyloxy)-4-methoxyphenyl]-3a,4,6,6a-tetrahydrofuro[3,4-d]isoxazole (Compound No. 44aa),
3-[3-(Cyclopentyloxy)-4-methoxyphenyl]-6,6a-dihydrofuro[3,4-d]isoxazol-4(3aH)-one (Compound No. 45aa),
Tert-butyl [({3-[3-(cyclopentyloxy)-4-methoxyphenyl]-1-oxa-2-azaspiro[4.5]dec-2-en-8-yl}amino)carbonyl]carbamate (Compound No. 46aa),
N-{3-[3-(Cyclopentyloxy)-4-methoxyphenyl]-1-oxa-2-azaspiro[4.5]dec-2-en-8-yl}cyclopentanecarboxamide (Compound No. 47aa),
8-Acetyl-3-[3-(cyclopentyloxy)-4-methoxyphenyl]-1-oxa-2,8-diazaspiro[4.5]dec-2-ene (Compound No. 48aa),
8-(Cyclopentylcarbonyl)-3-[3-(cyclopentyloxy)-4-methoxyphenyl]-1-oxa-2,8-diazaspiro[4.5]dec-2-ene (Compound No. 49aa),
3-[3-(Cyclopentyloxy)-4-methoxyphenyl]-8-(2-piperidin-1-ylethyl)-1-oxa-2,8-diazaspiro[4.5]dec-2-ene (Compound No. 50aa),
3-(2,3-Dihydro-1,4-benzodioxin-6-yl)-1,7-dioxa-2-azaspiro[4.4]non-2-ene (Compound No. 51aa),
3-[3-(Cyclopentyloxy)-4-methoxyphenyl]-1,8-dioxa-2-azaspiro[4.5]dec-2-ene (Compound No. 52aa),
3-[3-(Cyclopentyloxy)-4-methoxyphenyl]-3aH-cyclopenta[d]isoxazole-4,6(5H,6aH)-dione (Compound No. 53aa),
3-[3-(Cyclopentyloxy)-4-methoxyphenyl]-8-ethyl-1-oxa-2,8-diazaspiro[4.5]dec-2-ene (Compound No. 54aa),
3-[3-(Cyclopentyloxy)-4-methoxyphenyl]-8-vinyl-1-oxa-2-azaspiro[4.5]dec-2-en-8-ol (Compound No. 55aa),
3-[3-(Cyclopentyloxy)-4-methoxyphenyl]-3a,4,5,6,7,7a-hexahydro-1,2-benzisoxazole (Compound No. 56aa),
3-[3-(Cyclopentyloxy)-4-methoxyphenyl]-4,5,6,6a-tetrahydro-3aH-cyclopenta[d]isoxazole (Compound No. 57aa),
N-{3-[3-(Cyclopentyloxy)-4-methoxyphenyl]-1-oxa-2-azaspiro[4.5]dec-2-en-8-yl}methanesulfonamide (Compound No. 58aa),
3-[3-(Cyclopentyloxy)-4-methoxyphenyl]-8-methyl-1-oxa-2-azaspiro[4.5]dec-2-en-8-ol (Compound No. 59aa),
3-[3-(Allyloxy)-4-methoxyphenyl]-1,7-dioxa-2-azaspiro[4.4]non-2-ene (Compound No. 60aa),
3-[3-(2-Chloroethoxy)-4-methoxyphenyl]-1,7-dioxa-2-azaspiro[4.4]non-2-ene (Compound No. 61aa),
2-(Cyclopentyloxy)-4-(1,7-dioxa-2-azaspiro[4.4]non-2-en-3-yl)phenol (Compound No. 62aa),
3-(4-Butoxy-3-isobutoxyphenyl)-1,7-dioxa-2-azaspiro[4.4]non-2-ene (Compound No. 63aa),
3-(3-Isobutoxy-4-propoxyphenyl)-1,7-dioxa-2-azaspiro[4.4]non-2-ene (Compound No. 64aa),
3-[3-Butoxy-4-(cyclopropylmethoxy)phenyl]-1,7-dioxa-2-azaspiro[4.4]non-2-ene (Compound No. 65aa),
3-(3-Butoxy-4-ethoxyphenyl)-1,7-dioxa-2-azaspiro[4.4]non-2-ene (Compound No. 66aa),
3-[3-Butoxy-4-(cyclohexyloxy)phenyl]-1,7-dioxa-2-azaspiro[4.4]non-2-ene (Compound No. 67aa),
3-[3-(Cyclohexylmethoxy)-4-ethoxyphenyl]-1,7-dioxa-2-azaspiro[4.4]non-2-ene (Compound No. 68aa),
3-[3-(Cyclohexylmethoxy)-4-isopropoxyphenyl]-1,7-dioxa-2-azaspiro[4.4]non-2-ene (Compound No. 69aa),
3-[4-Butoxy-3-(cyclohexylmethoxy)phenyl]-1,7-dioxa-2-azaspiro[4.4]non-2-ene (Compound No. 70aa),
3-(4-Isobutoxy-3-isopropoxyphenyl)-1,7-dioxa-2-azaspiro[4.4]non-2-ene (Compound No. 71aa),
3-(4-Butoxy-3-isopropoxyphenyl)-1,7-dioxa-2-azaspiro[4.4]non-2-ene (Compound No. 72aa),
3-[4-(Cyclohexylmethoxy)-3-isopropoxyphenyl]-1,7-dioxa-2-azaspiro[4.4]non-2-ene (Compound No. 73aa),
3-[3-Isopropoxy-4-(2-morpholin-4-ylethoxy)phenyl]-1,7-dioxa-2-azaspiro[4.4]non-2-ene (Compound No. 74aa),
3-[3-(Cyclopropylmethoxy)-4-isopropoxyphenyl]-1,7-dioxa-2-azaspiro[4.4]non-2-ene (Compound No. 75aa),
3-[3-(Cyclopropylmethoxy)-4-(2-morpholin-4-ylethoxy)phenyl]-1,7-dioxa-2-azaspiro[4.4]non-2-ene (Compound No. 76aa),
3-[4-Butoxy-3-(cyclopropylmethoxy)phenyl]-1,7-dioxa-2-azaspiro[4.4]non-2-ene (Compound No. 77aa),
3-[3-(Cyclopropylmethoxy)-4-isopropoxyphenyl]-1,7-dioxa-2-azaspiro[4.4]non-2-ene (Compound No. 78aa),
3-(3-Isobutoxy-4-isopropoxyphenyl)-1,7-dioxa-2-azaspiro[4.4]non-2-ene (Compound No. 79aa),
3-[4-(Cyclopropylmethoxy)-3-isobutoxyphenyl]-1,7-dioxa-2-azaspiro[4.4]non-2-ene (Compound No. 80aa),
3-[4-(cyclohexyloxy)-3-(cyclopentyloxy)phenyl]-1,7-dioxa-2-azaspiro[4.4]non-2-ene (Compound No. 81aa),
3-[4-(Cyclohexylmethoxy)-3-(cyclopentyloxy)phenyl]-1,7-dioxa-2-azaspiro[4.4]non-2-ene (Compound No. 82aa),
3-[4-(Cyclopropylmethoxy)-3-(cyclopentyloxy)phenyl]-1,7-dioxa-2-azaspiro[4.4]non-2-ene (Compound No. 83aa),
3-[3-(Cyclopentyloxy)-4-isobutoxyphenyl]-1,7-dioxa-2-azaspiro[4.4]non-2-ene (Compound No. 84aa),
3-[3-(Cyclopentyloxy)-4-ethoxyphenyl]-1,7-dioxa-2-azaspiro[4.4]non-2-ene (Compound No. 85aa),
3-[3-(Cyclopropylmethoxy)-4-ethoxyphenyl]-1,7-dioxa-2-azaspiro[4.4]non-2-ene (Compound No. 86aa),
3-[4-(Cyclopentyloxy)-3-isobutoxyphenyl]-1,7-dioxa-2-azaspiro[4.4]non-2-ene (Compound No. 87aa),
3-[3-Isopropoxy-4-(2-morpholin-4-ylethoxy)phenyl]-1,7-dioxa-2-azaspiro[4.4]non-2-ene (Compound No. 88aa),
3-(4-Ethoxy-3-isobutoxyphenyl)-1,7-dioxa-2-azaspiro[4.4]non-2-ene (Compound No. 89aa),
3-[3-(Cyclopentyloxy)-4-propoxyphenyl]-1,7-dioxa-2-azaspiro[4.4]non-2-ene (Compound No. 90aa),
3-[4-Butoxy-3-(cyclopentyloxy)phenyl]-1,7-dioxa-2-azaspiro[4.4]non-2-ene (Compound No. 91aa)
3-[3-(Cyclopentyloxy)-4-isopropoxyphenyl]-1,7-dioxa-2-azaspiro[4.4]non-2-ene (Compound No. 92aa),
3-[3-(Cyclopentyloxy)-4-(cycloheptyloxy)phenyl]-1,7-dioxa-2-azaspiro[4.4]non-2-ene (Compound No. 93aa),
3-[3-(Cyclopentyloxy)-4-(2-morpholin-4-ylethoxy)phenyl]-1,7-dioxa-2-azaspiro[4.4]non-2-ene (Compound No. 94aa),
3-[4-(Cyclohexylmethoxy)-3-isobutoxyphenyl]-1,7-dioxa-2-azaspiro[4.4]non-2-ene (Compound No. 95aa),
3-[4-(Cyclohexylmethoxy)-3-(cyclopropylmethoxy)phenyl]-1,7-dioxa-2-azaspiro[4.4]non-2-ene (Compound No. 96aa),
3-[3-(Cyclopropylmethoxy)-4-propoxyphenyl]-1,7-dioxa-2-azaspiro[4.4]non-2-ene (Compound No. 97aa),
3-[4-(Cyclopentyloxy)-3-(cyclopropylmethoxy)phenyl]-1,7-dioxa-2-azaspiro[4.4]non-2-ene (Compound No. 98aa),
3-[4-(Cyclopropylmethoxy)-3-isopropoxyphenyl]-1,7-dioxa-2-azaspiro[4.4]non-2-ene (Compound No. 99aa),
3-[4-(Cyclopentyloxy)-3-isopropoxyphenyl]-1,7-dioxa-2-azaspiro[4.4]non-2-ene (Compound No. 100aa),
3-(3-Isopropoxy-4-propoxyphenyl)-1,7-dioxa-2-azaspiro[4.4]non-2-ene (Compound No. 10aa),
3-(4-Ethoxy-3-isopropoxyphenyl)-1,7-dioxa-2-azaspiro[4.4]non-2-ene (Compound No. 102aa),
3-[3-Butoxy-4-(2-morpholin-4-ylethoxy)phenyl]-1,7-dioxa-2-azaspiro[4.4]non-2-ene (Compound No. 103aa),
3-[3-Butoxy-4-(cyclopentyloxy)phenyl]-1,7-dioxa-2-azaspiro[4.4]non-2-ene (Compound No.
3-(3-Butoxy-4-propoxyphenyl)-1,7-dioxa-2-azaspiro[4.4]non-2-ene (Compound No. 105aa),
3-(3-Butoxy-4-isopropoxyphenyl)-1,7-dioxa-2-azaspiro[4.4]non-2-ene (Compound No. 106aa),
3-[3-(Cyclohexylmethoxy)-4-propoxyphenyl]-1,7-dioxa-2-azaspiro[4.4]non-2-ene (Compound No. 107aa),
3-[3-(Cyclohexylmethoxy)-4-isobutoxyphenyl]-1,7-dioxa-2-azaspiro[4.4]non-2-ene (Compound No. 108aa),
3-[3-(Cyclohexylmethoxy)-4-(cyclopentyloxy)phenyl]-1,7-dioxa-2-azaspiro[4.4]non-2-ene (Compound No. 109aa),
3-[3-(Cyclohexylmethoxy)-4-(cyclopropylmethoxy)phenyl]-1,7-dioxa-2-azaspiro[4.4]non-2-ene (Compound No. 110aa),
3-[4-(Cyclohexylmethoxy)-3-propoxyphenyl]-1,7-dioxa-2-azaspiro[4.4]non-2-ene (Compound No. 111aa),
3-[4-(Cyclopropylmethoxy)-3-propoxyphenyl]-1,7-dioxa-2-azaspiro[4.4]non-2-ene (Compound No. 112aa),
3-[4-(Cyclopentyloxy)-3-propoxyphenyl]-1,7-dioxa-2-azaspiro[4.4]non-2-ene (Compound No. 113aa),
3-[4-(3-Isobutoxy)-3-propoxyphenyl]-1,7-dioxa-2-azaspiro[4.4]non-2-ene (Compound No. 114aa),
3-[3-(Cycloheptyloxy)-4-(cyclopropylmethoxy)phenyl]-1,7-dioxa-2-azaspiro[4.4]non-2-ene (Compound No. 115aa),
3-[3-(Cycloheptyloxy)-4-propoxyphenyl]-1,7-dioxa-2-azaspiro[4.4]non-2-ene (Compound No. 116aa),
3-[4-Butoxy-3-(cycloheptyloxy)phenyl]-1,7-dioxa-2-azaspiro[4.4]non-2-ene (Compound No. 117aa),
3-[3-(Cycloheptyloxy)-4-isopropoxyphenyl]-1,7-dioxa-2-azaspiro[4.4]non-2-ene (Compound No. 118aa),
3-[3-(Cycloheptyloxy)-4-(cyclopentyloxy)phenyl]-1,7-dioxa-2-azaspiro[4.4]non-2-ene (Compound No. 119aa),
3-(3-Ethoxy-4-propoxyphenyl)-1,7-dioxa-2-azaspiro[4.4]non-2-ene (Compound No. 120aa),
3-[4-(Cycloheptyloxy)-3-ethoxyphenyl]-1,7-dioxa-2-azaspiro[4.4]non-2-ene (Compound No. 121aa),
3-[4-(Cyclopropylmethoxy)-3-ethoxyphenyl]-1,7-dioxa-2-azaspiro[4.4]non-2-ene (Compound No. 122aa),
3-[4-(Cyclohexylmethoxy)-3-ethoxyphenyl]-1,7-dioxa-2-azaspiro[4.4]non-2-ene (Compound No. 123aa),
(S)-3-[3-(cyclopentyloxy)-4-methoxyphenyl]-1,7-dioxa-2-azaspiro[4.4]non-2-ene (Compound No. 124aa),
3-(3-Butoxy-4-isobutoxyphenyl)-1,7-dioxa-2-azaspiro[4.4]non-2-ene (Compound No. 125aa),
3-(3-Ethoxy-4-isopropoxyphenyl)-1,7-dioxa-2-azaspiro[4.4]non-2-ene (Compound No. 126aa)
3-[4-(Cyclopentyloxy)-3-ethoxyphenyl]-1,7-dioxa-2-azaspiro[4.4]non-2-ene (Compound No. 127aa),
3-(4-Butoxy-3-ethoxyphenyl)-1,7-dioxa-2-azaspiro[4.4]non-2-ene (Compound No. 128aa),
3-(3-Ethoxy-4-isobutoxyphenyl)-1,7-dioxa-2-azaspiro[4.4]non-2-ene (Compound No. 129aa),
3-[3-(Cycloheptyloxy)-4-isobutoxyphenyl]-1,7-dioxa-2-azaspiro[4.4]non-2-ene (Compound No. 130aa),
3-[3-(Cycloheptyloxy-4-(cyclopentyloxy)phenyl]-1,7-dioxa-2-azaspiro[4.4]non-2-ene (Compound No. 131aa),
3-[3-(Cycloheptyloxy)-4-ethoxyphenyl]-1,7-dioxa-2-azaspiro[4.4]non-2-ene (Compound No. 132aa),
3-(4-Butoxy-3-propoxyphenyl)-1,7-dioxa-2-azaspiro[4.4]non-2-ene (Compound No. 133aa)
3-(4-Ethoxy-3-propoxyphenyl)-1,7-dioxa-2-azaspiro[4.4]non-2-ene (Compound No. 134aa),
3-[4-(Morpholin-4-ylethoxy)-3-propoxyphenyl]-1,7-dioxa-2-azaspiro[4.4]non-2-ene (Compound No. 135aa),
3-(4-Isopropoxy-3-propoxyphenyl)-1,7-dioxa-2-azaspiro[4.4]non-2-ene (Compound No. 136aa),
2-[5-(1,7-Dioxa-2-azaspiro[4.4]non-2-en-3-yl)-2-methoxyphenoxy]cyclopentanol (Compound No. 137aa),
N-{3-[3-(Cyclopentyloxy)-4-methoxyphenyl]-1-oxa-2-azaspiro[4.5]dec-2-en-8-yl}-2-fluorobenzamide (Compound No. 138aa),
N-{3-[3-(cyclopentyloxy)-4-methoxyphenyl]-1-oxa-2-azaspiro[4.5]dec-2-en-8-yl}benzamide (Compound No. 139aa),
3-[3-(Cyclopentyloxy)-4-methoxyphenyl]-4,5,6,6a-tetrahydro-3aH-pyrrolo[3,4-d]isoxazole (Compound No. 140aa),
7-(Cyclopentylcarbonyl)-3-[3-(cyclopentyloxy)-4-methoxyphenyl]1-oxa-2,7-diazaspiro[4.5]dec-2-ene (Compound No. 141aa),
Tert-butyl 3-[3-(cyclopentyloxy)-4-methoxyphenyl]-3a,4,6,6a-tetrahydro-5H-pyrrolo[3,4-d]isoxazole-5-carboxylate (Compound No. 142aa),
3-[3-(Cyclopentyloxy)-4-methoxyphenyl]-1-oxa-2,8-diazaspiro[4.5]dec-2-ene-8-carboxamide (Compound No. 143aa)
N-Butyl-3-[3-(cyclopentyloxy)-4-methoxyphenyl]-1-oxa-2,7-diazaspiro[4.5]dec-2-ene-7-carboxamide (Compound No. 144aa),
3-[3-(Cyclopentyloxy)-4-methoxyphenyl]-7-(methylsulfonyl)-1-oxa-2,7-diazaspiro[4.5]dec-2-ene (Compound No. 145aa),
3-[4-Methoxy-3-(pyridin-3-ylmethoxy)phenyl]-1,7-dioxa-2-azaspiro[4.4]non-2-ene (Compound No. 146aa),
5-Acetyl-3-[3-(cyclopentyloxy-4-methoxyphenyl]-4,5,6,6a-tetrahydro-3aH-pyrrolo[3,4-d]isoxazole (Compound No. 147aa),
3-[3-(Cyclopentyloxy)-4-methoxyphenyl]-5-(methylsulfonyl)-4,5,6,6a-tetrahydro-3aH-pyrrolo[3,4-d]isoxazole (Compound No. 148aa),
4-Bromo-3-[3-(cyclopentyloxy)-4-methoxyphenyl]-1,7-dioxa-2-azaspiro[4.4]non-2-ene (Compound No. 149aa),
3-[3-(Cyclopentyloxy)-4-methoxyphenyl]-3a,5,6,7a-tetrahydro-1,2-benzisoxazol-7(4H)-one (Compound No. 150aa),
3-[4-(Difluoromethoxy)-3-(2,3-dihydro-1H-inden-2-yloxy)phenyl]-1,7-dioxa-2-azaspiro[4.4]non-2-ene (Compound No. 151aa),
3-[4-(Cyclopentyloxy)-3-(2,3-dihydro-1H-inden-2-yloxy)phenyl]-1,7-dioxa-2-azaspiro[4.4]non-2-ene (Compound No. 152aa),
3-[4-Butoxy-3-(2,3-dihydro-1H-inden-2-yloxy)phenyl]-1,7-dioxa-2-azaspiro[4.4]non-2-ene (Compound No. 153aa)
3-(3-{[3-(Benzyloxy)cyclopentyl]oxy}-4-methoxyphenyl)-1,7-dioxa-2-azaspiro[4.4]non-2-ene (Compound No. 154aa),
7-Acetyl-3-[3-(cyclopentyloxy)-4-methoxyphenyl]-1-oxa-2,7-diazaspiro[4.5]dec-2-ene (Compound No. 155aa),
3-[4-Methoxy-3-(pyridin-2-ylmethoxy)phenyl]-1,7-dioxa-2-azaspiro[4.4]non-2-ene (Compound No. 156aa),
3-[3-(2,3-Dihydro-1H-inden-2-yloxy)-4-ethoxyphenyl]-1,7-dioxa-2-azaspiro[4.4]non-2-ene (Compound No. 157aa),
3-[3-(2,3-Dihydro-1H-inden-2-yloxy)-4-propoxyphenyl]-1,7-dioxa-2-azaspiro[4.4]non-2-ene (Compound No. 158aa),
3-[4-(Cyclopropylmethoxy)-3-(2,3-dihydro-1H-inden-2-yloxy phenyl]-1,7-dioxa-2-azaspiro[4.4]non-2-ene (Compound No. 159aa),
3-[3-(2,3-Dihydro-1H-inden-2-yloxy)-4-isopropoxyphenyl]-1,7-dioxa-2-azaspiro[4.4]non-2-ene (Compound No. 160aa),
2-(2,3-Dihydro-1H-inden-2-yloxy)-4-(1,7-dioxa-2-azaspiro[4.4]non-2-en-3-yl)phenol (Compound No. 161aa),
N-cyclopropyl-2-[5-(1,7-dioxa-2-azaspiro[4.4]non-2-en-3-yl)-2-methoxyphenoxy]acetamide (Compound No. 162aa),
Hydrochloride salt of 3-[4-methoxy-3-(piperidin-3-yloxy)phenyl]-1,7-dioxa-2-azaspiro[4.4]non-2-ene (Compound No. 163aa),
2-[5-(1,7-Dioxa-2-azaspiro[4.4]non-2-en-3-yl)-2-methoxyphenoxy]acetamide (Compound No. 164aa),
Ethyl [5-(1,7-dioxa-2-azaspiro[4.4]non-2-en-3-yl)-2-methoxyphenoxy]acetate (Compound No. 165aa),
[5-(1,7-Dioxa-2-azaspiro[4.4]non-2-en-3-yl)-2-methoxyphenoxy]acetonitrile (Compound No. 166aa , and
3-{3-[(2,6-Dichloropyridin-4-yl)methoxy]-4-methoxyphenyl}-1,7-dioxa-2-azaspiro[4.4]non-2-ene (Compound No. 167aa).

4. A method of treating or preventing autoimmune, inflammatory, or allergic disorders, wherein the method comprises administering to a mammal in need thereof a pharmaceutical composition comprising one or more muscarinic receptor antagonists (“MRA”) and at least one additional active ingredients selected from one or more β2-agonists, p38 MAP kinase inhibitors, PDE-IV inhibitors, corticosteroids, anticholinergics, dopamine agonists, antiallergics, PAF antagonists, leukotriene antagonists, EGFR kinase inhibitors, different muscarinic receptor antagonists or a mixture thereof, wherein the MRA has the structures of Formula I, II, or III, wherein
a. Formula I is: 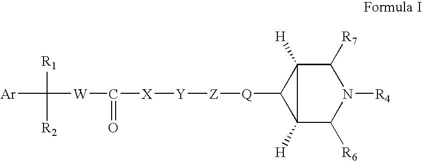
or a pharmaceutically acceptable salt, pharmaceutically acceptable solvate, ester, enantiomer, diastereomer, N-oxide, polymorphs, prodrugs or metabolite thereof, wherein
Ar represents an aryl or a heteroaryl ring having 1-2 heteroatoms independently selected from oxygen, sulphur or nitrogen, wherein
the aryl or heteroaryl ring may be unsubstituted or substituted by one to three substituents independently selected from lower alkyl (C₁-C₄), lower perhalo alkyl (C₁-C₄), cyano, hydroxy, nitro, lower alkoxy (C₁-C₄), lower perhalo alkoxy (C₁-C₄), unsubstituted amino, N-lower alkyl (C₁-C₄), N-aryl amino, amino carbonyl, N-lower alkyl (C₁-C₄) or N-aryl amino carbonyl;
R₁ represents hydrogen, hydroxy, hydroxy methyl, substituted or unsubstituted amino, alkoxy, carbamoyl or halogen (e.g., fluorine, chlorine, bromine and iodine);
R₂ represents alkyl, (C₃-C₇) cycloalkyl ring, (C₃-C₇) cycloalkenyl ring, aryl, heterocyclic ring, or heteroaryl ring, wherein
the heterocyclic ring or heteroaryl ring may have 1 to 2 heteroatoms independently selected from oxygen, sulphur or nitrogen, and
the aryl or heteroaryl ring may be unsubstituted or substituted by one to three substituents independently selected from lower alkyl (C₁-C₄), lower perhalo alkyl (C₁-C₄), cyano, hydroxy, nitro, lower alkoxycarbonyl, halogen, lower alkoxy (C₁-C₄), lower perhalo alkoxy (C₁-C₄), unsubstituted amino, N-lower alkyl (C₁-C₄) or N-aryl amino, amino carbonyl, N-lower alkyl (C₁-C₄) or N-aryl amino carbonyl;
W represents (CH₂)ₚ, wherein p represents 0 to 1;
X represents oxygen, sulphur, —NR or no atom (i.e., a bond), wherein
R represents hydrogen or (C₁₋₆) alkyl;
Y represents CHR₅CO or (CH₂)_{q}, wherein
R₅ represents hydrogen or methyl, and
q represents 0 to 4;
Z represents oxygen, sulphur, or NR₁₀, wherein
R₁₀ represents hydrogen, or C₁₋₆ alkyl;
Q represents (CH₂)ₙ, CHR₈ or CH₂CHR₉, wherein
n represents 0 to 4,
R₈ represents H, OH, C₁₋₆, alkyl, C₁₋₆ alkenyl, or C₁₋₆ alkoxy, and
R₉ represents H, OH, lower alkyl (C₁-C₄) or lower alkoxy (C₁-C₄);
R₆ and R₇ are independently selected from H, CH₃, COOH, CONH₁₂, NH₂ or CH₂NH₂; and
R₄ represents hydrogen or C₁-C₁₅ saturated or unsaturated aliphatic hydrocarbon group, wherein
1 to 6 hydrogen atoms of C₁-C₁₅ saturated or unsaturated aliphatic hydrocarbon group may be substituted with a group independently selected from halogen, arylalkyl, arylalkenyl, heteroarylalkyl or heteroarylalkenyl, wherein
heteroarylalkyl or heteroarylalkenyl may have 1 to 2 heteroatoms independently selected nitrogen, oxygen or sulphur, and
any 1 to 3 hydrogen atoms on the ring of arylalkyl, arylalkenyl, heteroarylalkenyl may be optionally substituted with lower alkyl (C₁-C₄), lower perhalo alkyl (C₁-C₄), cyano, hydroxyl, nitro, lower alkoxycarbonyl, halogen, lower alkoxy (C₁-C₄), lower perhaloalkoxy (C₁-C₄), unsubstituted amino, N-lower alkylamino (C₁-C₄), or N-lower alkylamino carbonyl (C₁-C₄);
b. Formula II is: 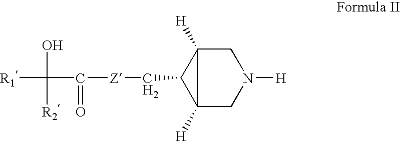
or a pharmaceutically acceptable salt, pharmaceutically acceptable solvate, ester, enantiomer, diastereomer, N-oxide, polymorph or metabolite thereof, wherein
R₁′ and R₂′ are independently selected from C₁-C₆ alkyl, C₃-C₇ cycloalkyl or phenyl, wherein phenyl is optionally substituted with one or more groups independently selected from C₁-C₃ alkyl, C₁-C₃ alkoxy or halogen; and
Z′ represents oxygen or NR₃, wherein
R₃ represents hydrogen or C₁-C₃ alkyl;
c. Formula III is, 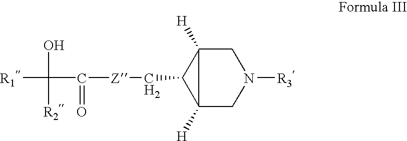
or a pharmaceutically acceptable salt, pharmaceutically acceptable solvate, ester, enantiomer, diastereomer, N-oxide, polymorph, prodrug or metabolite thereof, wherein
R₁″ and R₂″ are independently selected from C₁-C₆ alkyl, C₃-C₇ cycloalkyl, C₃-C₇ cycloalkenyl or phenyl, wherein phenyl is optionally substituted with one or more groups independently selected from C₁-C₃ alkyl, C₁-C₃ alkoxy or halogen;
R₃′ represents C₁-C₆ alkyl, wherein
1-3 hydrogen atom(s) may be substituted with a group independently selected from C₅-C₇ cycloalkyl, 1,3-dioxo-1,3-dihydro-isoindolyl or phenyl, wherein
phenyl is optionally substituted with one or more groups independently selected C₁-C₄ alkyl or halogen; and
Z represents oxygen or NR₄′, wherein
R₄′ represents hydrogen or C₁-C₃ alkyl.
